(19) United States
(12) Patent Application Publication
(10) Pub. No.: US 2026/0232566 A1
(43) Pub. Date: Aug. 13, 2026

(54) COSMETIC COMPOSITION COMPRISING A POLYHYDROXYALKANOATE COPOLYMER BEARING A(N) (UN)SATURATED HYDROCARBON-BASED CHAIN AND A CRYSTALLIZABLE FATTY SUBSTANCE

(71) Applicant: L'OREAL, Paris (FR)

(72) Inventors: Odile AUBRUN, Chevilly la Rue (FR); Angélina ROUDOT, Chevilly la Rue (FR); Jean-Yves FOURON, Aulnay-Sous-Bois (FR); Julien PORTAL, Aulnay-Sous-Bois (FR)

(73) Assignee: L'OREAL, Paris (FR)

(21) Appl. No.: 18/707,260

(22) PCT Filed: Dec. 22, 2022
(86) PCT No.: PCT/EP2022/087547

§ 371 Date: May 3, 2024

(30) Foreign Application Priority Data
Dec. 23, 2021 (FR) 2114378

(51) Int. Cl. A61K 8/85; A61K 8/37; A61Q 5/00; A61Q 19/00

(52) U.S. Cl. CPC A61K 8/85; A61K 8/37; A61K 8/375; A61Q 5/00; A61Q 19/001; A61K 2800/85

(57) ABSTRACT

The present invention relates to a cosmetic composition comprising
a) one or more polyhydroxyalkanoate (PHA) copolymers which contain, and preferably consist of, several repeating units chosen from the units (A) below, and also the optical or geometrical isomers thereof, the organic or mineral acid or base salts thereof, and the solvates thereof such as hydrates:

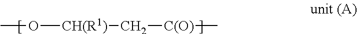

in which polymer units (A):
   R¹ are as defined in the description:
b) one or more crystallizable fatty substances.

The present invention relates to a cosmetic composition comprising a) at least one polyhydroxyalkanoate copolymer bearing (un)saturated hydrocarbon-based groups, b) at least one crystallizable fatty substance, c) optionally at least one oil, d) optionally at least one organic solvent and e) optionally water, and also to a process for treating keratin materials using such a composition.

It is known practice to use, in cosmetics, film-forming polymers which can be conveyed in organic media, such as hydrocarbon-based oils. Polymers are notably used as film-forming agents in makeup products such as mascaras, eyeliners, eyeshadows or lipsticks.

FR-A-2964663 describes a cosmetic composition comprising pigments coated with a C₃-C₂₁ polyhydroxyalkanoate, such as poly(hydroxybutyrate-co-hydroxyvalerate).

WO 2011/154508 describes a cosmetic composition comprising a 4-carboxy-2-pyrrolidinone ester derivative and a film-forming polymer which may be a polyhydroxyalkanoate, such as polyhydroxybutyrate, polyhydroxyvalerate and polyhydroxybutyrate-co-polyhydroxyvalerate.

US-A-2015/274972 describes a cosmetic composition comprising a thermoplastic resin, such as a polyhydroxyalkanoate, in aqueous dispersion and a silicone elastomer.

The majority of the polyhydroxyalkanoate copolymers are polymers derived from the polycondensation of polymeric repeating units that are for the most part identical and derived from the same carbon source or substrate. These documents do not describe the cosmetic use of copolymers derived from polycondensation using an aliphatic substrate or first carbon source, and at least one second substrate different from the first, comprising one or more (un)saturated hydrocarbon-based groups with polysaccharides. There is thus a need for a composition comprising polyhydroxyalkanoate copolymers which are lipophilic or soluble in a fatty phase. This makes it possible to obtain a film on keratin materials which has good cosmetic properties, notably good resistance to oils and to sebum, and also to be able to modify the gloss or the mattness.

The Applicant has discovered that polyhydroxyalkanoate copolymers bearing particular grafted or functionalized hydrocarbon-based groups, as defined below, may be readily used in fatty media, thus making it possible to obtain homogeneous compositions. Composition C1 shows good stability, notably after storage for one month at room temperature (25° C.). Composition C1, notably after its application to keratin materials, makes it possible to obtain a film having good cosmetic properties, good persistence of the colour without running for when the composition, and also a matt or glossy appearance of the treated keratin materials.

Crystallizable fatty substances, notably waxes, are known and widely used in cosmetics in the technology of deodorant sticks, lipsticks, eye shadows and mascaras, notably for the forming and moulding of sticks, as maintenance binders during compression for the formation of solid or semi-solid compositions. Cream skin cleansers notably for facial skin have been used for many years; in particular, conventional cold creams are composed of mineral oil (50% to 60%), beeswax (about 15%), borax (about 1%) and water (30% to 40%). Neutralization of the cerotic acid in the beeswax with borax gives the emulsifier to form the cream. Liquid and semi-solid glosses are formulated from mineral oils or plant or animal fats thickened with waxes (ozokerite), fatty alcohols (cetyl alcohol), or polymers (for example polyethylene), and are normally packaged in jars or tubes. Waxes are also used for hair removal in mixtures of waxes and resins (rosin and beeswax) mixed with lipids, for example oleyl oleate, which melt at a temperature of between 50° C. and 55° C. (Kirk-Othmer Encyclopedia of Chemical Technology. “Cosmetics”, M. and A. Rieger, et al., pages 1-49. Beeswaxes are also associated with dihydroabietyl alcohol for mascaras. However, the resistance to water and to rubbing is not always satisfactory.

In many conditions of use of film-forming materials on keratin materials, for instance in makeup or colouring applications, it is desirable to have, in addition to good resistance to water and oils, notably food oils such as olive oil, very good resistance to rubbing of the deposits of film-forming materials both to avoid transfer, for example onto clothing, and to maintain a homogeneous appearance of the deposits. If the resistance to rubbing is insufficient, the deposits obtained can quickly become very unsightly for consumers, in particular if these deposits are coloured as in makeup applications such as lipsticks, foundations or mascaras. In hair applications, the absence of resistance to rubbing is also very problematic in all colouring applications since it gives rise to transfer onto clothing and creates an unsightly appearance of the keratin fibres. There is thus a need to improve the persistence of PHAs conveyed in aqueous phase.

There is thus a real need to obtain deposits of film-forming materials that are resistant to oils, notably food oils, and that are water-resistant and have very good resistance to rubbing.

When the deposit is coloured, these problems of resistance to humidity lead to a transfer of colour, for example onto clothing, which is in itself problematic and which makes the deposit very unsightly.

These problems are solved by the use of the compositions C1 described hereinbelow, these compositions making it possible to significantly improve the resistance to rubbing of polyhydroxyalkanoate (PHA) copolymer(s). Furthermore, the compositions C1 according to the invention make it possible to obtain, after deposition, a film on keratin materials which has good cosmetic properties, notably good resistance to oils and to sebum, and good water resistance, and also to be able to modify the gloss or the mattness.

Thus, the main subject of the present invention is a composition C1 comprising:
a) one or more polyhydroxyalkanoate (PHA) copolymers which contain, and preferably consist of, several repeating units chosen from the units (A) below, and also the optical or geometrical isomers thereof, the organic or mineral acid or base salts thereof, and the solvates thereof such as hydrates:

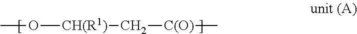

in which polymer units (A):
   R¹ represents a saturated or unsaturated, linear or branched, non-cyclic hydrocarbon-based chain, or a saturated or unsaturated, aromatic or non-aromatic cyclic hydrocarbon-based chain, comprising from 5 to 28 carbon atoms; preferably, the hydrocarbon-based chain is chosen from i) linear or branched (C₅-C₂₈)alkyl, ii) linear or branched (C₅-C₂₈)alkenyl, iii) linear or branched (C₅-C₂₈)alkynyl; preferably, the hydrocarbon-based group is linear;
      said hydrocarbon-based chain being:
         optionally substituted with one or more atoms or groups chosen from: a) halogen such as chlorine or bromine, b) hydroxyl, c) thiol, d) (di)(C₁-C₄)(alkyl)amino, e) (thio)carboxy, f) (thio)carboxamide —C(O)—N(Rₐ)₂ or C(S)—N(Rₐ)₂, g) cyano, h) iso(thio)cyanate, i) (hetero)aryl such as phenyl or furyl, and j) (hetero)cycloalkyl such as anhydride, epoxide or dithiolane, k) cosmetic active agent; l) R—X with R representing a group chosen from α) cycloalkyl such as cyclohexyl, β) heterocycloalkyl such as sugar, preferably monosaccharide such as glucose, γ) (hetero)aryl such as phenyl, δ) cosmetic active agent, m) thiosulfate, and X representing a′) O, S, N(Rₐ) or Si(R_{b})(R_{c}), b′) S(O)ᵣ, or (thio)carbonyl, c′) or combinations of a′) with b′) such as (thio)ester, (thio)amide, (thio)urea or sulfonamide; Rₐ representing a hydrogen atom, or a (C₁-C₄)alkyl group or an aryl(C₁-C₄)alkyl group such as benzyl; preferably, Rₐ represents a hydrogen atom; R_{b} and R_{c}, which may be identical or different, represent a (C₁-C₄)alkyl or (C₁-C₄)alkoxy group, particularly only one substituent; preferably chosen from b) halogen, and j) such as epoxide; and/or
         optionally interrupted with one or more a′) heteroatoms such as O, S, N(Rₐ) and Si(R_{b})(R_{c}), b′) S(O)ᵣ, (thio)carbonyl, c′) or combinations of a′) with b′) such as (thio)ester, (thio)amide, (thio)urea, sulfonamide with r being equal to 1 or 2, Rₐ being as defined previously; preferably, Rₐ represents a hydrogen atom, R_{b} and R_{c} being as defined previously;
         and
b) one or more fatty substances that are crystallizable at 25° C. and at atmospheric pressure; and
c) optionally one or more oils, i.e. fatty substances that are liquid at 25° C. and at atmospheric pressure; and
d) optionally one or more organic solvents, preferably polar aprotic or protic solvents, particularly polar and protic solvents;
e) optionally water; and preferably, composition C1 contains ingredient c) and preferably ingredients c)+d).

Another subject of the invention is the cosmetic use of a composition C1 comprising a) one or more PHA copolymers as defined previously, b) one or more crystallizable fatty substances as defined previously, optionally c) one or more fatty substances other than b) as defined previously, d) optionally one or more organic solvents as defined previously, and e) optionally water.

Another subject of the invention is a process for treating keratin materials, preferably α) keratin fibres, notably human keratin fibres such as the hair, or p) human skin, in particular the lips, using a) one or more PHA copolymers as defined previously, b) one or more crystallizable fatty substances, optionally c) one or more fatty substances other than b), optionally d) one or more organic solvents, and optionally e) water.

More particularly, a subject of the invention is a non-therapeutic cosmetic process for treating keratin materials, comprising the application to the keratin materials of a composition C1 as defined previously, or C1′ as defined hereinbelow. The treatment process is in particular a process for caring for or making up keratin materials.

For the purposes of the present invention and unless otherwise indicated:
the term “cosmetic active agent” means the radical of an organic or organosilicon compound which can be integrated into a cosmetic composition to give an effect on keratin materials, whether this effect is immediate or provided by repeated applications. As examples of cosmetic active agents, mention may be made of coloured or uncoloured, fluorescent or non-fluorescent chromophores such as those derived from optical brighteners, or chromophores derived from UVA and/or UVB screening agents, anti-ageing active agents or active agents intended for providing a benefit to the skin such as active agents having action on the barrier function, deodorant active agents other than mineral particles, antiperspirant active agents other than mineral particles, desquamating active agents, antioxidant active agents, moisturizing active agents, sebum-regulating active agents, active agents intended for limiting the sheen of the skin, active agents intended for combating the effects of pollution, antimicrobial or bactericidal active agents, antidandruff active agents, and fragrances.
the term “(hetero)aryl” means aryl or heteroaryl groups;
the term “(hetero)cycloalkyl” means cycloalkyl or heterocycloalkyl groups;
the “aryl” or “heteroaryl” radicals or the aryl or heteroaryl part of a radical may be substituted with at least one substituent borne by a carbon atom, chosen from:
   a C₁-C₆ and preferably C₁-C₄ alkyl radical;
   a halogen atom such as chlorine, fluorine or bromine;
   a hydroxyl group;
   a C₁-C₂ alkoxy radical; a C₂-C₄ (poly)hydroxyalkoxy radical;
   an amino radical;
   an amino radical substituted with one or two identical or different C₁-C₆ and preferably C₁-C₄ alkyl radicals;
   an acylamino radical (—NR—COR′) in which the radical R is a hydrogen atom;
   a C₁-C₄ alkyl radical and the radical R¹ is a C₁-C₄ alkyl radical; a carbamoyl radical ((R)₂N—CO—) in which the radicals R, which may be identical or different, represent a hydrogen atom or a C₁-C₄ alkyl radical;
   an alkylsulfonylamino radical (R′SO₂—NR—) in which the radical R represents a hydrogen atom or a C₁-C₄ alkyl radical and the radical R′ represents a C₁-C₄ alkyl radical, or a phenyl radical;
   an aminosulfonyl radical ((R)₂N—S(O)₂—) in which the radicals R, which may be identical or different, represent a hydrogen atom or a C₁-C₄ alkyl radical;
   a carboxylic radical in acid form or salified (preferably with an alkali metal or a substituted or unsubstituted ammonium) form;
   a cyano group (CN);
   a polyhalo(C₁-C₄)alkyl group, preferentially trifluoromethyl (CF₃);
the cyclic or heterocyclic part of a non-aromatic radical may be substituted with at least one substituent borne by a carbon atom, chosen from the groups:
   hydroxyl,
   C₁-C₄ alkoxy, C₂-C₄ (poly)hydroxyalkoxy;
   alkylcarbonylamino (RCO—NR′—), in which the radical R′ is a hydrogen atom or a C₁-C₄ alkyl radical and the radical R is a C₁-C₂ alkyl radical or an amino radical substituted with one or two identical or different C₁-C₄ alkyl groups;
   alkylcarbonyloxy (RCO—O—), in which the radical R is a C₁-C₄ alkyl radical or an amino radical substituted with one or two identical or different C₁-C₄ alkyl groups;
   alkoxycarbonyl (RO—CO—) in which the radical R is a C₁-C₄ alkyl radical or an amino radical substituted with one or two identical or different C₁-C₄ alkyl groups;
a cyclic or heterocyclic radical, or a non-aromatic part of an aryl or heteroaryl radical, may also be substituted with one or more oxo groups;
a hydrocarbon-based chain is unsaturated when it includes one or more double bonds and/or one or more triple bonds;
an “aryl” radical represents a monocyclic or fused or non-fused polycyclic hydrocarbon-based group comprising from 6 to 22 carbon atoms, and at least one ring of which is aromatic; preferentially, the aryl radical is a phenyl, biphenyl, naphthyl, indenyl, anthracenyl or tetrahydronaphthyl;
a “heteroaryl” radical represents a monocyclic or fused or non-fused polycyclic, 5- to 22-membered group, comprising from 1 to 6 heteroatoms chosen from nitrogen, oxygen, sulfur and selenium atoms, and at least one ring of which is aromatic; preferentially, a heteroaryl radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridyl, tetrazolyl, dihydrothiazolyl, imidazopyridyl, imidazolyl, indolyl, isoquinolyl, naphthoimidazolyl, naphthooxazolyl, naphthopyrazolyl, oxadiazolyl, oxazolyl, oxazolopyridyl, phenazinyl, phenoxazolyl, pyrazinyl, pyrazolyl, pyrilyl, pyrazoyltriazyl, pyridyl, pyridinoimidazolyl, pyrrolyl, quinolyl, tetrazolyl, thiadiazolyl, thiazolyl, thiazolopyridyl, thiazoylimidazolyl, thiopyrylyl, triazolyl and xanthylyl;
a “cyclic” or “cycloalkyl” radical is a monocyclic or fused or non-fused polycyclic, non-aromatic cyclic hydrocarbon-based radical containing from 5 to 22 carbon atoms, which may include one or more unsaturations; the cycloalkyl is preferably a cyclohexyl group;
a “heterocyclic” or “heterocycloalkyl” radical is a monocyclic or fused or non-fused polycyclic 3- to 9-membered non-aromatic cyclic radical, including from 1 to 4 heteroatoms chosen from nitrogen, oxygen, sulfur and selenium atoms; preferably, the heterocycloalkyl is chosen from epoxide, piperazinyl, piperidyl, morpholinyl and dithiolane;
an “alkyl” radical is a linear or branched, in particular C₁-C₆ and preferably C₁-C₄ saturated hydrocarbon-based radical;
an “alkenyl” radical is a linear or branched unsaturated hydrocarbon-based radical comprising one or more conjugated or non-conjugated double bonds;
an “alkynyl” radical is a linear or branched unsaturated hydrocarbon-based radical comprising one or more conjugated or non-conjugated triple bonds;
an “alkoxy” radical is an alkyl-oxy radical for which the alkyl radical is a linear or branched C₁-C₆ and preferentially C₁-C₄hydrocarbon-based radical;
a “sugar” radical is a monosaccharide or polysaccharide radical, and the O-protected sugar derivatives thereof such as sugar esters of (C₁-C₆)alkylcarboxylic acids such as acetic acid, sugars containing amine group(s) and (C₁-C₄)alkyl derivatives, such as methyl derivatives, for instance methylglucose. Sugar radicals that may be mentioned include: sucrose, glucose, galactose, ribose, fucose, maltose, fructose, mannose, arabinose, xylose, lactose;
the term “monosaccharide” refers to a monosaccharide sugar comprising at least 5 carbon atoms of formula Cₓ(H₂O)ₓ with x an integer greater than or equal to 5, preferably x is greater than or equal to 6, in particular x is between 5 and 7 inclusive, preferably x=6; they may be of D or L configuration, and of alpha or beta anomer, and also the salts thereof and the solvates thereof such as hydrates;
the term “polysaccharide” refers to a polysaccharide sugar which is a polymer constituted of several saccharides bonded together via O-oside bonds, said polymers being constituted of monosaccharide units as defined previously, said monosaccharide units comprising at least 5 carbon atoms, preferably 6; in particular, the monosaccharide units are linked together via a 1,4 or 1,6 bond as a (alpha) or p (beta) anomer, it being possible for each oside unit to be of L or D configuration, and also the salts thereof and the solvates thereof such as the hydrates of said monosaccharides; more particularly, they are polymers formed from a certain number of saccharides (or monosaccharides) having the general formula: —[Cₓ(H₂O)_{y})]_{w}— or —[(CH₂O)ₓ]_{w}—, where x is an integer greater than or equal to 5, preferably x is greater than or equal to 6, in particular x is between 5 and 7 inclusive and preferably x=6, and y is an integer which represents x−1, and w is an integer greater than or equal to 2, particularly between 3 and 3000 inclusive, more particularly between 5 and 2500, preferentially between 10 and 2300, particularly between 15 and 1000 inclusive, more particularly between 20 and 500, preferentially between 25 and 200;
the term “sugar bearing amine group(s)” means that the sugar radical is substituted with one or more amino groups NR₁R₂, i.e. at least one of the hydroxyl groups of at least one saccharide unit of the sugar radical is replaced with a group NR₁R₂, with R₁ and R₂, which may be identical or different, representing i) a hydrogen atom, ii) a (C₁-C₆)alkyl group, iii) an aryl group such as phenyl, iv) an aryl(C₁-C₄)alkyl group such as benzyl, vii) —C(Y)—(Y′)_{f}—R′₁ with Y and Y′, which may be identical or different, representing an oxygen atom, a sulfur atom or N(R′₂), preferably oxygen, f=0 or 1, preferably 0; and R′₂ and R′₂ representing i) to vi) of R₁ and R₂ defined previously, and in particular R′₁ denoting a (C₁-C₆)alkyl group such as methyl. Preferably, R₁ and/or R₂ represent a hydrogen atom, or a (C₁-C₄)alkylcarbonyl group such as acetyl, and more preferentially R₁ represents a hydrogen atom and R₂ represents a (C₁-C₄)alkylcarbonyl group such as acetyl;
the term “organic or mineral acid salt” more particularly means organic or mineral acid salts in particular chosen from a salt derived from i) hydrochloric acid HCl, ii) hydrobromic acid HBr, iii) sulfuric acid H₂SO₄, iv) alkylsulfonic acids: Alk-S(O)₂OH such as methanesulfonic acid and ethanesulfonic acid; v) arylsulfonic acids: Ar—S(O)₂OH such as benzenesulfonic acid and toluenesulfonic acid; vi) alkoxysulfinic acids: Alk-O—S(O)OH such as methoxysulfinic acid and ethoxysulfinic acid; vii) aryloxysulfinic acids such as tolueneoxysulfinic acid and phenoxysulfinic acid; viii) phosphoric acid H₃PO₄; ix) triflic acid CF₃SO₃H and x) tetrafluoroboric acid HBF₄; xi) organic carboxylic acids R^{o}—C(O)—OH (I′z), in which formula (I′z) R^{o} represents a (hetero)aryl group such as phenyl, (hetero)aryl(C₁-C₄)alkyl group such as benzyl, or (C₁-C₁₀)alkyl, said alkyl group being optionally substituted preferably with one or more hydroxyl groups or amino or carboxyl radicals, R^{o} preferably denoting a (C₁-C₆)alkyl group optionally substituted with 1, 2 or 3 hydroxyl or carboxyl groups; more preferentially, the monocarboxylic acids of formula (I′z) are chosen from acetic acid, glycolic acid, lactic acid, and mixtures thereof, and more particularly from acetic acid and lactic acid; and the polycarboxylic acids are chosen from tartaric acid, succinic acid, fumaric acid, citric acid and mixtures thereof; and xii) amino acids including more carboxylic acid radicals than amino groups, such as γ-carboxyglutamic acid, aspartic acid or glutamic acid, in particular γ-carboxyglutamic acid;
an “anionic counterion” is an anion or an anionic group associated with the cationic charge; more particularly, the anionic counterion is chosen from: i) halides such as chloride or bromide; ii) nitrates; iii) sulfonates, including C₁-C₆ alkylsulfonates: Alk-S(O)₂O⁻ such as methylsulfonate or mesylate and ethylsulfonate; iv) arylsulfonates: Ar—S(O)₂O⁻ such as benzenesulfonate and toluenesulfonate or tosylate; v) citrate; vi) succinate; vii) tartrate; viii) lactate; ix) alkyl sulfates: Alk-O—S(O)O— such as methyl sulfate and ethyl sulfate; x) aryl sulfates: Ar—O—S(O)O— such as benzene sulfate and toluene sulfate; xi) alkoxy sulfates: Alk-O—S(O)₂O— such as methoxy sulfate and ethoxy sulfate; xii) aryloxy sulfates: Ar—O—S(O)₂O—; xiii) phosphate; xiv) acetate; xv) triflate; and xvi) borates such as tetrafluoroborate;
the “solvates” represent hydrates and also the combination with linear or branched C₁-C₄ alcohols such as ethanol, isopropanol or n-propanol;
the term “chromophore” means a radical derived from a colourless or coloured compound that is capable of absorbing in the UV and/or visible radiation range at a wavelength λ_{abs} of between 250 and 800 nm. Preferably, the chromophore is coloured, i.e. it absorbs wavelengths in the visible range, i.e. preferably between 400 and 800 nm. Preferably, the chromophores appear coloured to the eye, particularly between 400 and 700 nm (Ullmann's Encyclopedia, 2005, Wiley-VcH, Verlag “Dyes, General Survey”, § 2.1 Basic Principle of Color);
the term “fluorescent chromophore” means a chromophore which is also capable of re-emitting in the visible range at an emission wavelength λₑₘ of between 400 and 800 nm, and higher than the absorption wavelength, preferably with a Stoke's shift, i.e. the difference between the maximum absorption wavelength and the emission wavelength is at least 10 nm. Preferably, fluorescent chromophores are derived from fluorescent dyes that are capable of absorbing in the visible range λ_{abs}, i.e. at a wavelength of between 400 and 800 nm, and of re-emitting in the visible range at a λₑₘ of between 400 and 800 nm. More preferentially, fluorescent chromophores are capable of absorbing at a λ_{abs} of between 420 and 550 nm and of re-emitting in the visible range λₑₘ of between 470 and 600 nm;
the term “optical brightening chromophore” means a chromophore derived from an optical brightening compound or “optical brighteners, optical brightening agents (OBAs)” or “fluorescent brightening agents (FBAs)” or “fluorescent whitening agents (FWAs)”, i.e. agents which absorb UV radiation, i.e. at a wavelength λ_{abs} of between 250 and 350 nm, and of subsequently re-emitting this energy by fluorescence in the visible range at an emission wavelength λₑₘ of between 400 and 600 nm, i.e. wavelengths between blue-violet and blue-green with a maximum in the blue range. Optical brightening chromophores are thus colourless to the eye;
the term “UV-A screening agent” means a chromophore derived from a compound which screens out (or absorbs) UV-A ultraviolet rays at a wavelength of between 320 and 400 nm. A distinction may be made between short UV-A screening agents (which absorb rays at a wavelength of between 320 and 340 nm) and long UV-A screening agents (which absorb rays at a wavelength of between 340 and 400 nm);
the term “UV-B screening agent” means a chromophore derived from a compound which screens out (or absorbs) UV-B ultraviolet rays at a wavelength of between 280 and 320 nm.
   Furthermore, unless otherwise indicated, the limits delimiting the extent of a range of values are included in that range of values.

### a) The PHA Copolymer(s)

Composition C1 of the invention comprises as first ingredient a) one or more PHA copolymers which contain, or preferably consist of, at least two different repeating polymer units (A) as defined previously.

According to a particular embodiment of the invention, composition C1′ according to the invention comprises as first ingredient a) one or more PHA copolymers which contain, or preferably consist of, at least two different repeating polymer units chosen from the units (A) and (B) as defined previously.

Preferably, composition C of the invention is a composition C1′, preferably a cosmetic composition comprising:
a) one or more polyhydroxyalkanoate (PHA) copolymers which contain, and preferably consist of, at least two different repeating polymer units chosen from the units (A) and (B) below, and also the optical or geometrical isomers thereof, the organic or mineral acid or base salts thereof, and the solvates thereof such as hydrates:

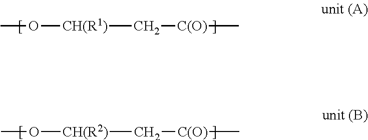

in which polymer units (A) and (B):
   R¹ represents a saturated or unsaturated, linear or branched, non-cyclic hydrocarbon-based chain, or a saturated or unsaturated, aromatic or non-aromatic cyclic hydrocarbon-based chain, comprising from 5 to 28 carbon atoms; particularly a hydrocarbon-based chain chosen from i) linear or branched (C₅-C₂₈)alkyl, ii) linear or branched (C₅-C₂₈)alkenyl, iii) linear or branched (C₅-C₂₈)alkynyl; preferably, the hydrocarbon-based group is linear;
      said hydrocarbon-based chain being:
         optionally substituted with one or more atoms or groups chosen from: a) halogen such as chlorine or bromine, b) hydroxyl, c) thiol, d) (di)(C₁-C₄)(alkyl)amino, e) (thio)carboxy, f) (thio)carboxamide —C(O)—N(Rₐ)₂ or C(S)—N(Rₐ)₂, g) cyano, h) iso(thio)cyanate, i) (hetero)aryl such as phenyl or furyl, and j) (hetero)cycloalkyl such as anhydride, epoxide or dithiolane, k) cosmetic active agent; l) R—X with R representing a group chosen from α) cycloalkyl such as cyclohexyl, β) heterocycloalkyl such as sugar, preferably monosaccharide such as glucose, γ) (hetero)aryl such as phenyl, δ) cosmetic active agent, m) thiosulfate, and X representing a′) O, S, N(Rₐ) or Si(R_{b})(R_{c}), b′) S(O)ᵣ, or (thio)carbonyl, c′) or combinations of a′) with b′) such as (thio)ester, (thio)amide, (thio)urea or sulfonamide; Rₐ representing a hydrogen atom, or a (C₁-C₄)alkyl group or an aryl(C₁-C₄)alkyl group such as benzyl; preferably, Rₐ represents a hydrogen atom; R_{b} and R_{c}, which may be identical or different, represent a (C₁-C₄)alkyl or (C₁-C₄)alkoxy group, particularly only one substituent; preferably chosen from b) halogen, and j) such as epoxide; and/or
         optionally interrupted with one or more a′) heteroatoms such as O, S, N(Rₐ) and Si(R_{b})(R_{c}), b′) S(O)ᵣ, (thio)carbonyl, c′) or combinations of a′) with b′) such as (thio)ester, (thio)amide, (thio)urea or sulfonamide with r being equal to 1 or 2, Rₐ being as defined previously; preferably, Rₐ represents a hydrogen atom, R_{b} and R_{c} being as defined previously;
         R² represents a cyclic or non-cyclic, linear or branched, saturated or unsaturated hydrocarbon-based group comprising from 3 to 30 carbon atoms optionally substituted with one or more atoms or groups a) to m) and/or optionally interrupted with one or more heteroatoms or groups a′) to c′) as defined for R¹; in particular chosen from linear or branched (C₃-C₂₈)alkyl and linear or branched (C₃-C₂₈)alkenyl, in particular a linear hydrocarbon-based group, more particularly (C₄-C₂₀)alkyl or (C₄-C₂₀)alkenyl; preferably, the hydrocarbon-based group has a carbon number corresponding to the number of carbon atoms of the radical R¹ from which at least one carbon atom is subtracted, preferably corresponding to the number of carbon atoms of the radical R¹ from which two carbon atoms are subtracted; and
b) one or more fatty substances which are crystallizable at 25° C. and at atmospheric pressure; and
c) optionally one or more fatty substances other than b), which are preferably liquid at 25° C. and at atmospheric pressure; and
d) optionally, one or more organic solvents other than c);
e) optionally water;
it being understood that:
   (A) is different from (B); and
   composition C1 contains c) one or more fatty substances and e) water; preferably, composition C1 contains ingredients c)+d).

The term “co-polymer” means that said polymer is derived from the polycondensation of polymeric repeating units that are different from each other, i.e. said polymer is derived from the polycondensation of polymeric repeating units (A) that are different from each other, or from the polycondensation of polymeric repeating units (A) with (B), it being understood that the polymeric units (A) are different from the polymeric units (B), it being possible for said copolymer to be obtained from a single saturated or unsaturated aliphatic carbon source which is optionally substituted and/or interrupted, preferably unsubstituted and uninterrupted, or from several carbon sources, in particular at least one of which is an uninterrupted unsubstituted saturated aliphatic and the other carbon source(s) are saturated or unsaturated aliphatic, optionally substituted notably with a halogen atom such as bromine, or with a cyano group, a Bunte salt, a dithiolane radical, a carboxyl, etc.

According to one embodiment, the copolymer according to the invention is derived from a single carbon source, preferably a single saturated or unsaturated aliphatic carbon source which is optionally substituted and/or interrupted, preferably unsubstituted and uninterrupted.

According to one embodiment, the copolymer according to the invention is derived from several carbon sources, preferably from 2 to 10 carbon sources, more preferentially 2 to 5 carbon sources and even more preferentially 2 carbon sources.

According to one embodiment, the copolymer according to the invention is derived from several carbon sources and at least one is saturated aliphatic. According to a particular embodiment of the invention, the PHA copolymer(s) consist of two different repeating polymer units chosen from the units (A) and (B) as defined previously.

According to a particular embodiment of the invention, the PHA copolymer(s) contain or consist of two different repeating polymer units chosen from the units (A) as defined previously, the units (B) such that R² represents a cyclic or non-cyclic, linear or branched, saturated or unsaturated hydrocarbon-based group comprising from 3 to 30 carbon atoms; in particular chosen from linear or branched (C₃-C₂₈)alkyl and linear or branched (C₃-C₂₈)alkenyl, in particular a linear hydrocarbon-based group, more particularly (C₄-C₂₀)alkyl or (C₄-C₂₀)alkenyl; preferably, the hydrocarbon-based group has a carbon number corresponding to the number of carbon atoms of the radical R¹ from which at least one carbon atom is subtracted, preferably corresponding to the number of carbon atoms of the radical R¹ from which two carbon atoms are subtracted.

More particularly, the PHA copolymer(s) according to the invention comprise the repeating unit of formula (I), and also the optical or geometrical isomers thereof, the organic or mineral acid or base salts thereof, and the solvates thereof such as hydrates:

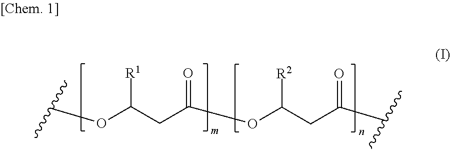

in which formula (I):
   R¹ and R² are as defined previously;
   m and n are integers greater than or equal to 1; preferably, the sum n+m is inclusively between 450 and 1400;
   preferably, m>n when R¹ and R² represent an unsubstituted and uninterrupted alkyl group—more preferentially, when R¹ and R² are linear alkyl, then R¹ is a C₅-C₁₃ alkyl group; and R² represents a linear alkyl group with a carbon number corresponding to the carbon number of R¹ from which two carbon atoms are subtracted such as a C₃-C₁₁ alkyl group; and
   preferably, m<n when R¹ represents a substituted and/or interrupted alkyl group, an optionally substituted and/or interrupted alkenyl group or an optionally substituted and/or interrupted alkynyl group, and R² represents an alkyl group.

According to a particular embodiment, the PHA copolymer(s) of composition a) contain three different repeating polymer units (A), (B) and (C), and preferably consist of three different polymer units (A), (B) and (C) below, and also the optical or geometrical isomers thereof and the solvates thereof such as hydrates:

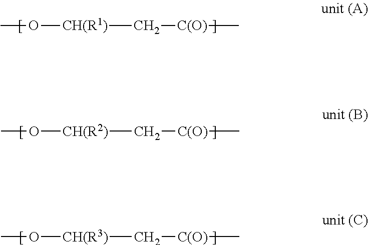

in which polymer units (A), (B) and (C):
   R¹ and R² are as defined previously;
   R³ represents a saturated or unsaturated, linear or branched, cyclic or non-cyclic, hydrocarbon-based group comprising from 1 to 30 carbon atoms, optionally substituted with one or more atoms or groups a) to m) and/or optionally interrupted with one or more heteroatoms or groups a′) to c′) as defined for R¹; it in particular represents a hydrocarbon-based group chosen from linear or branched (C₁-C₂₈)alkyl, and linear or branched (C₂-C₂₈)alkenyl, in particular a linear hydrocarbon-based group, more particularly (C₄-C₂₀)alkenyl; preferably, the hydrocarbon-based group has a carbon number corresponding to the number of carbon atoms in the radical R¹, or else corresponding to the number of carbon atoms in the radical R¹ minus at least three carbon atoms, preferably corresponding to the number of carbon atoms in the radical R¹ minus four carbon atoms; and
it being understood that:
   (A) is different from (B) and (C), (B) is different from (A) and (C), and (C) is different from (A) and (B); and
   preferably, when R¹, R² and R³ represent an unsubstituted and uninterrupted alkyl group, the molar percentage of units (A) is greater than the molar percentage of units
   (B), and greater than the molar percentage of units (C)—more preferentially, when R¹, R² and R³ are linear alkyl, then R¹ is a C₅-C₁₃ alkyl group; and R² represents a linear alkyl group with a carbon number corresponding to the carbon number of R¹ from which two carbon atoms are subtracted, and R³ represents a linear alkyl group with a carbon number corresponding to the carbon number of R¹ from which four carbon atoms are subtracted; and
   preferably, when R¹ represents a substituted and/or interrupted alkyl, optionally substituted and/or optionally interrupted alkenyl or optionally substituted and/or optionally interrupted alkynyl group, then the molar percentage of units (A) is less than the molar percentage of units (B) and the molar percentage of units (C) is less than the molar percentage of units (B) if R² represents an alkyl group and/or R³ represents an alkyl group.

According to a particular embodiment of the invention, the PHA copolymer(s) comprise the repeating unit of formula (II), and also the optical or geometrical isomers thereof, the organic or mineral acid or base salts thereof, and the solvates thereof such as hydrates:

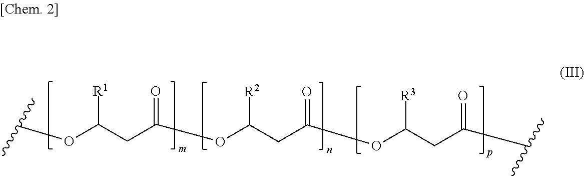

in which formula (II):
   R¹, R² and R³ are as defined previously;
   m, n and p are integers greater than or equal to 1; preferably, the sum n+m+p is inclusively between 450 and 1400; and
   preferably, m>n+p when R¹, R² and R³ represent an unsubstituted and uninterrupted alkyl group—more preferentially, when R¹, R² and R³ are linear alkyl, then R¹ is a C₅-C₁₃ alkyl group; and R² represents a linear alkyl group with a carbon number corresponding to the carbon number of R¹ from which two carbon atoms are subtracted such as a C₃-C₁₁ alkyl group, and R³ represents a linear alkyl group with a carbon number corresponding to the carbon number of R¹ from which four carbon atoms are subtracted such as a C₁-C₉ alkyl group; and
   preferably, m<n+p when R¹ represents a substituted and/or interrupted alkyl group, an optionally substituted and/or optionally interrupted alkenyl group or an optionally substituted and/or optionally interrupted alkynyl group, and R² and R³ represent an alkyl group.

According to a particular embodiment, the PHA copolymer(s) of composition a) contain four different repeating polymer units (A), (B), (C) and (D), and preferably consist of four different polymer units (A), (B), (C) and (D), below, and also the optical or geometrical isomers thereof, the organic or mineral acid or base salts thereof, and the solvates thereof such as hydrates:

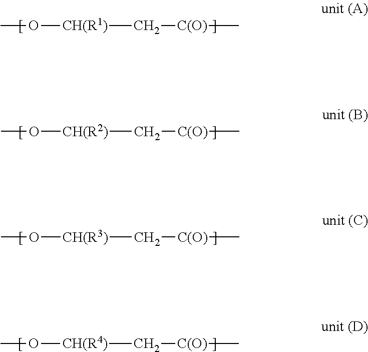

in which polymer units (A), (B), (C) and (D):
   R¹, R² and R³ are as defined previously;
   R⁴ represents a cyclic or non-cyclic, linear or branched, saturated hydrocarbon-based group comprising from 3 to 30 carbon atoms optionally substituted with one or more atoms or groups a) to m) and/or optionally interrupted with one or more heteroatoms or groups a′) to c′) as defined for R¹; it in particular represents a hydrocarbon-based group chosen from linear or branched (C₄-C₂₈)alkyl optionally substituted with one or more atoms or groups a) to m) and/or interrupted with one or more heteroatoms or groups a′) to c′) as defined for R¹; and
it being understood that:
   (A) is different from (B), (C) and (D), (B) is different from (A), (C) and (D), (C) is different from (A), (B) and (D), and (D) is different from (A), (B) and (C); and
   preferably, when R¹, R², R³ and R⁴ represent an unsubstituted and uninterrupted alkyl group, the molar percentage of units (A) is greater than the molar percentage of units
   (B), greater than the molar percentage of units (C), and greater than the molar percentage of units (D)—more preferentially, when R¹, R², R³ and R⁴ are linear alkyl, then R¹ is a C₅-C₁₃ alkyl group; and R² represents a linear alkyl group with a carbon number corresponding to the carbon number of R¹ from which two carbon atoms are subtracted such as a C₃-C₁₁ alkyl group, R³ represents a linear alkyl group with a carbon number corresponding to the carbon number of R¹ from which four carbon atoms are subtracted such as a C₁-C₉ alkyl group, and R⁴ represents a linear alkyl group with a carbon number corresponding to the carbon number of R¹ from which six carbon atoms are subtracted; and
   preferably, when R¹ represents a substituted and/or interrupted alkyl, optionally substituted and/or optionally interrupted alkenyl or optionally substituted and/or optionally interrupted alkynyl group, then the molar percentage of units (A) is less than the molar percentage of units (B), the molar percentage of units (C) is less than the molar percentage of units (B), notably if R² represents an alkyl group and/or R³ represents an alkyl group; and R⁴ represents an optionally substituted and/or optionally interrupted alkyl, optionally substituted and/or optionally interrupted alkenyl or optionally substituted and/or optionally interrupted alkynyl group.

According to a particular embodiment of the invention, the PHA copolymer(s) comprise the repeating unit of formula (III), and also the optical or geometrical isomers thereof, the organic or mineral acid or base salts thereof, and the solvates thereof such as hydrates:

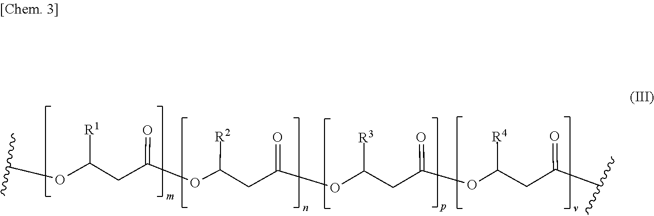

in which formula (III):
   R¹, R², R³ and R⁴ are as defined previously;
   m, n, p and v are integers greater than or equal to 1;
preferably, the sum n+m+p+v is inclusively between 450 and 1400; and
preferably, when R¹, R², R³ and R⁴ represent an unsubstituted and uninterrupted alkyl group, then m>n+p+q—more preferentially, when R¹, R², R³ and R⁴ are linear alkyl, then R¹ is a C₅-C₁₃ alkyl group; and R² represents a linear alkyl group with a carbon number corresponding to the carbon number of R¹ from which two carbon atoms are subtracted, R³ represents a linear alkyl group with a carbon number corresponding to the carbon number of R¹ from which four carbon atoms are subtracted, and R⁴ represents a linear alkyl group with a carbon number corresponding to the carbon number of R¹ from which six carbon atoms are subtracted; and
preferably, when R¹ represents a substituted and/or interrupted alkyl, optionally substituted and/or optionally interrupted alkenyl or optionally substituted and/or optionally interrupted alkynyl group, and R² and R³ represent an alkyl group, and R⁴ represents an optionally substituted and/or optionally interrupted alkyl, optionally substituted and/or optionally interrupted alkenyl or optionally substituted and/or optionally interrupted alkynyl group, then n>m+v; more preferentially n+p>m+v.

According to one embodiment, the PHA copolymer(s) of composition a) more particularly contain five different repeating polymer units (A), (B), (C), (D) and (E), and preferably consist of five different polymer units (A), (B), (C), (D) and (E), below, and also the optical or geometrical isomers thereof, the organic or mineral acid or base salts thereof, and also the solvates thereof such as hydrates:

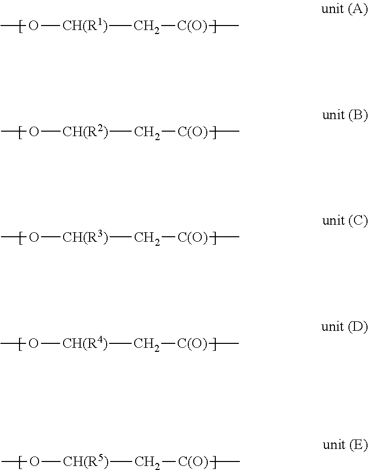

in which polymer units (A), (B), (C), (D) and (E):
   R¹, R², R³ and R⁴ are as defined previously; and
   R⁵ represents a cyclic or non-cyclic, linear or branched, saturated hydrocarbon-based group comprising from 3 to 30 carbon atoms optionally substituted with one or more atoms or groups a) to m) and/or optionally interrupted with one or more heteroatoms or groups a′) to c′) as defined for R¹; it in particular represents a hydrocarbon-based group chosen from linear or branched (C₄-C₂₈)alkyl optionally substituted with one or more atoms or groups a) to m) and/or interrupted with one or more heteroatoms or groups a′) to c′) as defined for R¹; preferably, the hydrocarbon-based group has a carbon number corresponding to the number of carbon atoms of the radical R⁴ from which at least one carbon atom is subtracted, preferably corresponding to the number of carbon atoms of the radical R⁴ from which at least two carbon atoms are subtracted, preferably from which two carbon atoms are subtracted;
it being understood that:
   (A) is different from (B), (C), (D) and (E); (B) is different from (A), (C), (D) and (E); (C) is different from (A), (B), (D) and (E); (D) is different from (A), (B), (C) and (E); and (E) is different from (A), (B), (C) and (D); and
   preferably, when R¹, R², R³, R⁴ and R⁵ represent an unsubstituted and uninterrupted alkyl group, the molar percentage of units (A) is greater than the molar percentage of units (B), greater than the molar percentage of units (C), greater than the molar percentage of units (D) and greater than the molar percentage of units (E)—more preferentially, when R¹, R², R³, R⁴ and R⁵ are linear alkyl, then R¹ is a C₅-C₁₃ alkyl group; and R² represents a linear alkyl group with a carbon number corresponding to the carbon number of R¹ from which two carbon atoms are subtracted, R³ represents a linear alkyl group with a carbon number corresponding to the carbon number of R¹ from which four carbon atoms are subtracted, R⁴ represents a linear alkyl group with a carbon number corresponding to the carbon number of R¹ from which six carbon atoms are subtracted, and R⁵ represents a linear alkyl group with a carbon number corresponding to the carbon number of R¹ from which eight carbon atoms are subtracted; and
   preferably, when R¹ represents a substituted and/or interrupted alkyl, optionally substituted and/or optionally interrupted alkenyl or optionally substituted and/or optionally interrupted alkynyl group, then the molar percentage of units (A) is less than the molar percentage of units (B), the molar percentage of units (C) is less than the molar percentage of units (B), notably if R² represents an alkyl group and/or R³ represents an alkyl group, and R⁴ and R⁵ represent a substituted and/or interrupted alkyl, optionally substituted and/or optionally interrupted alkenyl or optionally substituted and/or optionally interrupted alkynyl group.

According to a particular embodiment of the invention, the PHA copolymer(s) comprise the repeating unit of formula (IV), and also the optical or geometrical isomers thereof, the organic or mineral acid or base salts thereof, and the solvates thereof such as hydrates:

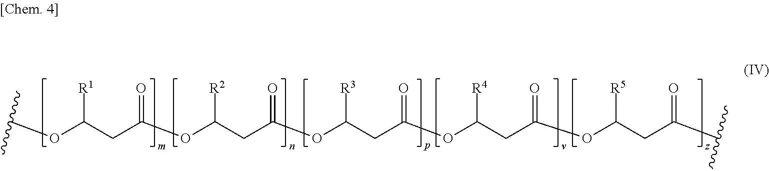

in which formula (IV):
   R¹, R², R³, R⁴ and R⁵ are as defined previously;
   m, n, p, v and z are integers greater than or equal to 1; preferably, the sum n+m+p+v+z is inclusively between 450 and 1400; and
preferably, when R¹, R², R³, R⁴ and R⁵ represent an unsubstituted and uninterrupted alkyl group, then m>n+p+v+z;
preferably, when R¹ represents a substituted and/or interrupted alkyl; optionally substituted and/or optionally interrupted alkenyl; or optionally substituted and/or optionally interrupted alkynyl group, R² and R³ represent an alkyl group, and the groups R⁴ and R⁵ represent a substituted and/or interrupted alkyl; optionally substituted and/or optionally interrupted alkenyl; or optionally substituted and/or optionally interrupted alkynyl group, then n>m+v+z; more preferentially n+p>m+v+z.

Preferably, R¹ represents a linear or branched, preferably linear, (C₅-C₂₈)alkyl hydrocarbon-based chain. According to one embodiment of composition C1 or C1′ according to the invention, the PHA copolymer(s) are such that the radical R¹ is an alkyl group comprising 5 to 14 and preferably between 6 and 12 carbon atoms, more preferentially between 7 and 10 carbon atoms such as n-pentyl, n-hexyl, n-octyl or n-nonyl.

According to a particular embodiment of the invention, the hydrocarbon-based chain R¹ is unsubstituted. According to a particular embodiment of the invention, the hydrocarbon-based chain R¹ is uninterrupted.

According to another embodiment, the hydrocarbon-based chain of the radical R¹ of the invention is 1) either substituted, 2) or interrupted, 3) or substituted and interrupted.

According to a particular embodiment of the invention, the PHA copolymer(s) are such that R¹ represents a hydrocarbon-based chain, notably an alkyl group as defined previously, which is interrupted with one or more (preferably one) atoms or groups chosen from O, S, N(Rₐ) and carbonyl, or combinations thereof such as ester, amide or urea, with Rₐ being as defined previously, preferably Rₐ represents a hydrogen atom; preferably, R¹ represents an alkyl group which is interrupted with one or more atoms chosen from O and S, more preferentially with an O or S, notably S, atom. In particular, when it represents an interrupted hydrocarbon-based chain, notably alkyl, R¹ is C₇-C₂₀, more particularly C₈-C₁₈ and even more particularly C₉-C₁₆. Preferably, said interrupted hydrocarbon-based chain, notably alkyl, is linear.

According to another embodiment of the invention, the PHA copolymer(s) are such that R¹ represents a hydrocarbon-based chain, notably an alkyl group as defined previously, substituted with one or more (preferably one) atoms or groups chosen from: a) to k) as defined previously. Preferably, said hydrocarbon-based chain is substituted with only one atom or group chosen from: a) to k) as defined previously.

According to a particular embodiment of the invention, the PHA copolymer(s) are such that R¹ represents a hydrocarbon-based chain, notably an alkyl group as defined previously, which is substituted with one or more (preferably one) groups chosen from a) halogen such as chlorine or bromine, b) hydroxyl, c) thiol, d) (di)(C₁-C₄)(alkyl)amino and preferably amino, e) carboxyl, i) (hetero)cycloalkyl such as anhydride, dithiolane or epoxide, j) a cosmetic active agent chosen from coloured or uncoloured, fluorescent or non-fluorescent chromophores such as optical brighteners, UV-screening agents, h) (hetero)aryl such as phenyl or furyl, k) R—X with R representing a group chosen from α) cycloalkyl such as cyclohexyl, β) heterocycloalkyl such as a sugar radical, preferably a monosaccharide such as glucosyl, γ) (hetero)aryl such as phenyl, δ) a cosmetic active agent as defined previously, m) thiosulfate and X representing a′) O, S, N(Rₐ), b′) carbonyl, c′) or combinations thereof of a′) with b′) such as ester, amide or urea; Rₐ represents a hydrogen atom or a (C₁-C₄)alkyl or aryl(C₁-C₄)alkyl group such as benzyl, preferably Rₐ represents a hydrogen atom.

Even more preferentially, the PHA copolymer(s) are such that R¹ represents a hydrocarbon-based chain, notably an alkyl group as defined previously, which is substituted with one or more (preferably one) groups chosen from a) halogen such as chlorine or bromine, b) hydroxyl, d) (di)(C₁-C₄)(alkyl)amino, preferably amino, e) carboxyl, i) (hetero)cycloalkyl such as epoxide, h) (hetero)aryl such as phenyl or furyl, k) R—X with R representing a group chosen from α) cycloalkyl such as cyclohexyl, β) heterocycloalkyl such as a sugar radical, preferably a monosaccharide such as glucosyl, γ) (hetero)aryl such as phenyl, and X representing a′) O, S or N(Rₐ), preferably S; Rₐ representing a hydrogen atom or a (C₁-C₄)alkyl group, preferably Rₐ represents a hydrogen atom.

According to one embodiment, said substituted hydrocarbon-based chain, notably alkyl, is linear.

According to another embodiment, said substituted hydrocarbon-based chain, notably alkyl, is branched.

According to another particular embodiment of the invention, the hydrocarbon-based chain of the radical R¹ of the invention is substituted and interrupted.

According to a particular embodiment of the invention, the hydrocarbon-based chain (notably an alkyl group as defined previously) of the radical R¹ of the invention is:
substituted with one or more (preferably one) groups chosen from a) halogen such as chlorine or bromine, b) hydroxyl, c) thiol, d) (di)(C₁-C₄)(alkyl)amino and preferably amino, e) carboxyl, i) (hetero)cycloalkyl such as anhydride, dithiolane or epoxide, j) a cosmetic active agent chosen from coloured or uncoloured, fluorescent or non-fluorescent chromophores such as optical brighteners, UV-screening agents, h) (hetero)aryl such as phenyl or furyl, k) R—X with R representing a group chosen from α) cycloalkyl such as cyclohexyl, β) heterocycloalkyl such as a sugar, preferably a monosaccharide such as glucose, γ) (hetero)aryl such as phenyl, δ) a cosmetic active agent as defined previously and X representing a′) O, S, N(Rₐ), b′) carbonyl, c′) or combinations thereof of a′) with b′) such as ester, amide or urea; Rₐ representing a hydrogen atom or a (C₁-C₄)alkyl or aryl(C₁-C₄)alkyl group such as benzyl, preferably Rₐ represents a hydrogen atom; and
interrupted with one or more (preferably one) atoms or groups chosen from O, S, N(Rₐ) and carbonyl, or combinations thereof such as ester, amide or urea, with Rₐ being as defined previously, preferably Rₐ represents a hydrogen atom; preferably an alkyl group which is interrupted with one or more atoms chosen from O and S, more preferentially with an O or S, notably S, atom. In particular, when it represents an interrupted hydrocarbon-based chain, notably alkyl, R¹ is C₇-C₂₀, more particularly C₈-C₁₈ and even more particularly C₉-C₁₆.

According to a preferred embodiment of the invention, the hydrocarbon-based chain (notably an alkyl group as defined previously) of the radical R¹ of the invention is:
substituted with one or more (preferably one) groups chosen from a) halogen such as chlorine or bromine, b) hydroxyl, d) (di)(C₁-C₄)(alkyl)amino, preferably amino, e) carboxyl, i) (hetero)cycloalkyl such as epoxide, h) (hetero)aryl such as phenyl or furyl, k) R—X with R representing a group chosen from α) cycloalkyl such as cyclohexyl, β) heterocycloalkyl such as a sugar, preferably a monosaccharide such as glucose, γ) (hetero)aryl such as phenyl, and X representing a′) O, S or N(Rₐ), preferably S; Rₐ representing a hydrogen atom or a (C₁-C₄)alkyl group, preferably Rₐ represents a hydrogen atom; and
interrupted with one or more (preferably one) atoms or groups chosen from O, S, N(Rₐ) and carbonyl, or combinations thereof such as ester, amide or urea, with Rₐ being as defined previously, preferably Rₐ represents a hydrogen atom; preferably an alkyl group which is interrupted with one or more atoms chosen from O and S, more preferentially with an O or S, notably S, atom. In particular, when it represents an interrupted hydrocarbon-based chain, notably alkyl, R¹ is C₇-C₂₀, more particularly C₈-C₁₈ and even more particularly C₉-C₁₆.

Preferably, said substituted and interrupted hydrocarbon-based chain is notably alkyl, and is preferably linear.

More preferentially, when said hydrocarbon-based chain R¹ is substituted, it is substituted at the end of the chain on the opposite side from the carbon atom which bears said radical R¹.

According to one embodiment of the invention, said hydrocarbon-based chain R¹ has the following formula —(CH₂)ᵣ—X-(ALK)ᵤ-G with X being as defined previously, in particular representing O, S or N(Rₐ), preferably S, ALK represents a linear or branched, preferably linear, (C₁-C₁₀)alkylene and more particularly (C₁-C₈)alkylene chain, r represents an integer inclusively between 6 and 11, preferably between 7 and 10 such as 8; u is equal to 0 or 1; and G represents a hydrogen atom or a group chosen from hydroxyl, carboxyl, (di)(C₁-C₄)(alkyl)amino, (hetero)aryl in particular aryl such as phenyl, cycloalkyl such as cyclohexyl, or a sugar, in particular a monosaccharide optionally protected with one or more groups such as acyl, preferably Sug.

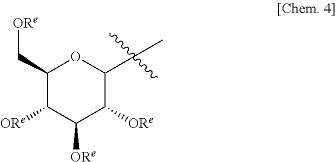

with R^{e} representing a group R^{f}—C(O)—, with R^{f} representing a (C₁-C₄)alkyl group such as methyl. Preferably, when u is equal to 0, G represents a cycloalkyl group such as cyclohexyl, or a sugar as defined previously. According to another advantageous variant, when u is equal to 1, G represents a hydrogen atom or a group chosen from hydroxyl, carboxyl, (di)(C₁-C₄)(alkyl)amino or (hetero)aryl, in particular aryl such as phenyl.

According to another particular embodiment of the invention, the PHA copolymer(s) are such that R¹ represents (C₅-C₂₈)alkyl substituted with one or more halogen atoms such as fluorine, chlorine or bromine, more particularly linear (C₅-C₂₀)alkyl, even more particularly (C₅-C₁₃)alkyl, substituted with a halogen atom such as bromine. Preferably, the halogen atom is substituted at the end of said alkyl group. More preferentially, R¹ represents 1-halo-5-yl such as 1-bromo-5-yl.

According to another particular embodiment of the invention, the PHA copolymer(s) are such that R¹ represents a (C₅-C₂₈)alkyl group substituted with one or more groups chosen from g) cyano, and more particularly represents a (C₅-C₁₃)alkyl group, which is preferably linear, substituted with a cyano group g), such as 1-cyano-3-propyl.

According to another particular embodiment of the invention, the PHA copolymer(s) are such that R¹ represents vii) a (hetero)aryl(C₁-C₂)alkyl and more particularly aryl(C₁-C₂)alkyl group, preferably phenylethyl.

According to another particular embodiment of the invention, the PHA copolymer(s) are such that R¹ represents a (C₅-C₂₈)alkyl group substituted with one or more groups chosen from c) (hetero)cycloalkyl. More particularly, R¹ represents a (C₅-C₁₃)alkyl group, which is preferably linear, substituted with a heterocycloalkyl group such as epoxide or dithiolane, preferably epoxide.

In particular, the PHA copolymer(s) are such that R² is chosen from linear or branched (C₁-C₂₈)alkyl, and linear or branched (C₂-C₂₈)alkenyl, in particular a linear hydrocarbon-based group, more particularly (C₃-C₂₀)alkyl or (C₃-C₂₀)alkenyl, preferably linear or branched, and more particularly linear, (C₃-C₂₀)alkyl.

In particular, the PHA copolymer(s) are such that R² is chosen from linear or branched (C₁-C₂₈)alkyl, and linear or branched (C₂-C₂₈)alkenyl, in particular a linear hydrocarbon-based group, more particularly (C₃-C₂₀)alkyl or (C₃-C₂₀)alkenyl; preferably, the hydrocarbon-based group has a carbon number corresponding to the number of carbon atoms of the radical R¹ from which at least one carbon atom is subtracted, preferably corresponding to the number of carbon atoms of the radical R¹ from which two carbon atoms are subtracted.

According to one embodiment of the invention, the PHA copolymer(s) are such that the radical R² is a linear or branched, preferably linear, (C₃-C₈)alkyl, in particular (C₃-C₆)alkyl, preferably (C₄-C₆)alkyl group such as n-pentyl or n-hexyl.

According to another embodiment of composition C1 or C1′ according to the invention, the PHA copolymer(s) comprise a branched (C₃-C₈)alkyl, particularly (C₄-C₆)alkyl radical R², preferably a branched (C₄-C₅)alkyl radical such as isobutyl.

According to another embodiment of composition C1 or C1′ according to the invention, the PHA copolymer(s) of the invention comprise the units (A) bearing an alkyl radical R¹ as defined previously, the units (B) as defined previously and the units (C) bearing a linear or branched (C₆-C₂₀)alkenyl, particularly (C₇-C₁₄)alkenyl and more particularly (C₈-C₁₀)alkenyl radical, which is preferably linear and comprising only one unsaturation at the chain end, in particular, —[CR⁴(R⁵)]_{q}—C(R⁶)═C(R⁷)—R⁸ with R⁴, R⁵, R⁶, R⁷ and R⁸, which may be identical or different, representing a hydrogen atom or a (C₁-C₄)alkyl group such as methyl, preferably a hydrogen atom, and q represents an integer inclusively between 2 and 20, preferably between 3 and 10, more preferentially between 4 and 8 such as 6, such as —[CH₂]_{q}—CH═CH₂ and q represents an integer inclusively between 3 and 8, preferably between 4 and 6, such as 5.

According to one embodiment of composition C1 or C1′ according to the invention, the PHA copolymer(s) comprise units (A) bearing an alkyl radical R¹ comprising between 8 and 16 carbon atoms substituted with one or more (preferably one) groups chosen from hydroxyl, (di)(C₁-C₄)(alkyl)amino, carboxyl, and R—X— as defined previously, preferably R—S— with R representing a cycloalkyl group such as cyclohexyl, heterocycloalkyl such as a sugar, more preferentially a monosaccharide such as glucose, optionally substituted aryl(C₁-C₄)alkyl such as (C₁-C₄)(alkyl)benzyl or phenylethyl, or heteroaryl(C₁-C₄)alkyl such as furylmethyl.

According to one embodiment of composition C1 or C1′ according to the invention, the copolymer(s) comprise units (B) bearing a linear or branched, preferably linear, (C₁-C₈)alkyl, particularly (C₂-C₆)alkyl, preferably (C₄-C₅)alkyl radical R² such as pentyl.

According to another embodiment of composition C1 or C1′ according to the invention, the PHA copolymer(s) comprise units (A) containing an alkyl radical R¹ as defined previously, units (B) as defined previously and units (C) containing a linear or branched (C₆-C₂₀)alkenyl, particularly (C₇-C₁₄)alkenyl radical and more particularly (C₈-C₁₀)alkenyl radical, which is preferably linear, and comprising only one unsaturation at the chain end such as —[CH₂]_{q}—CH═CH₂ and p represents an integer inclusively between 3 and 8, preferably between 4 and 6, such as 5.

According to a particular embodiment of the invention, in the PHA copolymer(s), the unit (A) comprises a hydrocarbon-based chain as defined previously, in particular ii), said unit (A) preferably being present in a molar percentage ranging from 0.1% to 99%, more preferentially a molar percentage ranging from 0.5% to 50%, even more preferentially a molar percentage ranging from 1% to 40%, better still a molar percentage ranging from 2% to 30%, or a molar percentage ranging from 5% to 20%.

According to a particular embodiment of the invention, in the PHA copolymer(s), the unit (A) is preferably present in a molar percentage ranging from 0.5% to 99%.

According to one embodiment, when R¹ represents a (C₅-C₂₈)alkyl group, the unit (A) is preferably present in a molar percentage ranging from 0.5% to 99%, more preferentially from 50% to 99%, more particularly from 60% to 99% and even more preferentially from 70% to 99%. According to this embodiment, the unit (B) is preferably present in a molar percentage ranging from 2% to 40%; and the unit (C) is preferably present in a molar percentage ranging from 0.5% to 20% relative to all the units (A), (B) and (C).

According to another embodiment, when R¹ represents a hydrocarbon-based chain chosen from i) linear or branched (C₅-C₂₈)alkyl, ii) linear or branched (C₅-C₂₈)alkenyl, iii) linear or branched (C₅-C₂₈)alkynyl, preferably the hydrocarbon-based group is linear, said hydrocarbon-based chain being substituted with one or more atoms or groups a) to m) and/or interrupted with one or more heteroatoms or groups a′) to c′) as defined for R¹; it in particular represents a hydrocarbon-based group chosen from linear or branched (C₄-C₂₈)alkyl, optionally substituted with one or more atoms or groups a) to m) and/or interrupted with one or more heteroatoms or groups a′) to c′) as defined previously, the unit (A) is preferably present in a molar percentage ranging from 0.5% to 99%, more preferentially a molar percentage ranging from 1% to 50%, even more preferentially a molar percentage ranging from 5% to 40%, better still a molar percentage ranging from 5% to 30%; the unit (B) is present in a molar percentage ranging from 1% to 99.5%, preferably from 1% to 99%, more preferentially from 2% to 98%, and the unit (C) is present in a molar percentage ranging from 0.5% to 20% relative to all the units (A), (B) and (C). Advantageously, the PHA copolymer(s) of the invention comprise from 2 mol % to 98 mol % of units (B), and from 0.5 mol % to 10 mol % of units (C); more advantageously, the copolymer comprises from 5 mol % to 70 mol % of units (B), and from 0.5 mol % to 7 mol % of units (C).

According to a more particular embodiment of the invention, the PHA copolymer(s) are such that, in the PHA copolymer(s) a):
the unit (A) comprises a hydrocarbon-based chain with one or more atoms or groups a) to m) and/or interrupted with one or more heteroatoms or groups a′) to c′) as defined previously, said unit (A) being present in a molar percentage ranging from 0.1% to 99%, preferentially a molar percentage ranging from 0.5% to 50%, more preferentially a molar percentage ranging from 1% to 40%, even more preferentially a molar percentage ranging from 2% to 30%, better still a molar percentage ranging from 5 mol % to 30 mol % of units (A); and
the unit (B) is present in a molar percentage ranging from 1% to 99.9%, preferentially a molar percentage from 2% to 99%, more preferentially a molar percentage from 5 mol % to 70 mol % of units (B); and/or
the unit (C) is present in a molar percentage ranging from 0.5% to 20%, preferentially a molar percentage from 1% to 10%, more preferentially from 0.5 mol % to 7 mol % of units (C).

Preferably, when R¹ of the unit (A) is a saturated unsubstituted and uninterrupted hydrocarbon-based chain, said unit (A) is present in a molar percentage of greater than 30%, more particularly greater than 50%, more preferentially greater than 60%, preferably between 60% and 90%.

The values of the molar percentages of the units (A), (B) and (C) of the PHA copolymer(s) are calculated relative to the total number of moles of (A)+(B) if the copolymer(s) do not comprise any additional units (C); otherwise, if the copolymer(s) of the invention contain three different units (A), (B) and (C), then the molar percentage is calculated relative to the total number of moles (A)+(B)+(C); otherwise, if the copolymer(s) of the invention contain four different units (A), (B), (C) and (D), then the molar percentage is calculated relative to the total number of moles (A)+(B)+(C)+(D); otherwise, if the copolymer(s) of the invention contain five different units (A), (B), (C), (D) and (E), then the molar percentage is calculated relative to the total number of moles (A)+(B)+(C)+(D)+(E).

According to one form of the invention, the PHA copolymer(s) of the invention comprise the following repeating units (A), and also the optical or geometrical isomers thereof, the organic or mineral acid or base salts thereof, and the solvates thereof such as hydrates:

| (A) | R¹ |
|---|---|
| A1 | -ALK₁-S-ALK₂-COOH |
| A2 | -ALK₁-S-ALK₂-H |
| A3 | -ALK₁-S-ALK₂-OH |
| A4 | -ALK₁-S-ALK₂-NH₂ |
| A5 | -ALK₁-S-Cycl′ |
| A6 | -ALK₁-S-CH₂-Fur |
| A7 | -ALK₁-S-Sug |
| A8 | -ALK₁-S-ALK₂-Ar |
| A9 | -ALK₁-Hal |
| A10 | -ALK₁-CN |
| A11 | -ALK₁-CH═CRᵣR_{w} |
| A12 | -ALK₂-H |

in which repeating units A1 to A12:
   ALK₁ represents a divalent linear or branched C₁-C₂₀, preferably linear or branched, more preferentially linear, C₁-C₁₀, hydrocarbon-based radical;
   ALK₂ represents a divalent linear or branched C₁-C₂₀, preferably linear or branched C₁-C₁₂, hydrocarbon-based radical;
   Rr and Rw independently denote a hydrogen atom or a C₁-C₄ alkyl radical such as methyl; preferably, Rr and Rw are identical;
   Hal represents a halogen atom such as bromine;
   Ar: represents a (hetero)aryl group such as phenyl;
   Cycl′: represents a cycloalkyl group such as cyclohexyl or heterocycloalkyl such as dithiolane, or epoxide, preferably epoxide;
   Fur: represents a furyl group, preferably 2-furyl;
   Sug: represents a sugar group, in particular a monosaccharide optionally protected with one or more groups such as acyl, in particular acetyl.

In particular, the stereochemistry of the carbon atoms bearing the radicals R¹ is of (R) configuration.

According to one form of the invention, the PHA copolymer(s) of the invention comprise the repeating units (B) of formula (A12), and also the optical or geometrical isomers thereof, the organic or mineral acid or base salts thereof, and the solvates thereof such as hydrates, it being understood that (B) is different from (A).

Preferentially, the PHA copolymer(s) of the invention comprise the following repeating units, and also the optical or geometrical isomers thereof, the organic or mineral acid or base salts thereof, and the solvates thereof such as hydrates:

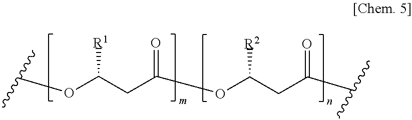

| Com- | | |
|---|---|---|
| pounds | R¹ | R² |
| (1) | —(CH₂) [symbol] —S—CH(CH₃)—C(O)—OH | —(CH₂)₄—CH₃ |
| (2) | —(CH₂)₈—S—(CH₂)₇—CH₃ | —(CH₂)₄—CH₃ |
| (3) | —(CH₂)₈—S—(CH₂)₈—OH | —(CH₂)₄—CH₃ |
| (4) | —(CH₂)₈—S—(CH₂)₂—NH₂ | —(CH₂)₄—CH₃ |
| (5) | —(CH₂)₈—S-Cycl | —(CH₂)₄—CH₃ |
| (6) | —(CH₂)₈—S—CH₂-Fur | —(CH₂)₄—CH₃ |
| (7) | —(CH₂)₈—S-Sug | —(CH₂)₄—CH₃ |
| (8) | —(CH₂)₈—S—(CH₂)₂—Ar | —(CH₂)₄—CH₃ |
| (9) | —(CH₂) [symbol] —S—CH₂—Ar′ | —(CH₂)₄—CH₃ |
| (10) | —(CH₂)₈—S—CH(CH₃)—C(O)—OH | —(CH₂) [symbol] —CH₃ |
| (11) | —(CH₂)₅-Hal | —(CH₂)₅—CH₃ |
| (12) | —(CH₂)₃—CN | —(CH₂)₅—CH₃ |
| (13) | 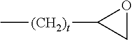 | —(CH₂)₅—CH₃ |
| (14) | —(CH₂)₂—Ar | —(CH₂)₅—CH₃ |
| (15) | —(CH₂)₄—CH₃ | —(CH₂)₂—CH₃ |
| (16) | —(CH₂)₅—CH₃ | —(CH₂)₃—CH₃ |
| (17) | —(CH₂)₆—CH₃ | —(CH₂)₄—CH₃ |
| (18) | —(CH₂)₈—CH₃ | —(CH₂)₆ [symbol] —CH₃ |
| (19) | —(CH₂)₃—CH(CH₃)CH₃ | —CH₂—CH(CH₃)CH₃ |
| (20) | —(CH₂)₆—CH═CH₂ | —(CH₂)₅—CH₃ |
| (21) | —(CH₂)₂—CH═C(CH₃)CH₃ | —CH₂—CH(CH₃)CH₃ |
| (2a) | —(CH₂)₈—S—(CH₂)₇—CH₃ | (CH₂)₅—(CH₃) |

[symbol]

indicates data missing or illegible when filed

m and n are as defined previously, Hal represents a halogen atom such as bromine and t represents an integer between 1 and 10, preferably between 3 and 8 such as 6.
Ar: represents a (hetero)aryl group such as phenyl;
Ar′: represents a (C₁-C₄)alkyl(hetero)aryl group such as t-butylphenyl, preferably 4-t-butylphenyl;
Cycl: represents a cyclohexyl group;
Fur: represents a furyl group, preferably 2-furyl;
Sug: represents a sugar group, in particular a monosaccharide optionally protected with one or more groups such as acyl; preferably, Sug represents:

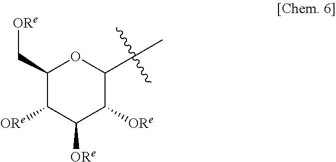

with R^{e} representing a group R^{f}—C(O)—, with R^{f} representing a (C₁-C₄)alkyl group such as methyl.

In particular, the stereochemistry of the carbon atoms bearing the radicals R¹ and R² is of the same (R) or (S) configuration, preferably of (R) configuration.

More particularly, the stereochemistry of the carbon atoms bearing the radicals R¹, R² and R³ is of the same (R) or (S) configuration, preferably of (R) configuration. More particularly, the stereochemistry of the carbon atoms bearing the radicals R¹, R², R³ and R⁴ is of the same (R) or (S) configuration, preferably of (R) configuration.

More particularly, the stereochemistry of the carbon atoms bearing the radicals R¹, R², R³, R⁴ and R⁵ is of the same (R) or (S) configuration, preferably of (R) configuration.

More preferentially, the PHA copolymer(s) have the following formula, and also the optical isomers thereof, the organic or mineral acid or base salts thereof, and the solvates thereof such as hydrates:

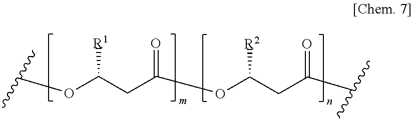

| Compounds | R¹ | R² |
|---|---|---|
| (1′) | —(CH₂ [symbol] S—CH(CH₃)—C(O)—OH | —(CH₂)₄—CH₃ |
| (2′) | —(CH₂)₈—S—(CH₂)₇—CH₃ | —(CH₂)₄—CH₃ |
| (3′) | —(CH₂)₈—S—(CH₂)₈—OH | —(CH₂)₄—CH₃ |
| (4′) | —(CH₂)₈—S—(CH₂)₂—NH₂ | —(CH₂)₄—CH₃ |
| (5′) | —(CH₂)₈—S-Cycl | —(CH₂)₄—CH₃ |
| (6′) | —(CH₂)₈—S—CH₂-Fur | —(CH₂)₄—CH₃ |
| (7′) | —(CH₂) [symbol] —S-Sug | —(CH₂)₄—CH₃ |
| (8′) | —(CH₂) [symbol] —S—(CH₂)₂-Ar | —(CH₂)₄—CH₃ |
| (9′) | —(CH₂) [symbol] —S—CH₂—Ar′ | —(CH₂)₄—CH₃ |
| (10′) | —(CH₂)₈—S—CH(CH₃)—C(O)—OH | —(CH₂)₅—CH₃ |
| (11′) | —(CH₂)₈-Hal | —(CH₂)₅—CH₃ |
| (12′) | —(CH₂)₃—CN | —(CH₂)₅—CH₃ |
| (13′) | 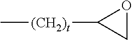 | —(CH₂) [symbol] —CH₃ |
| (14′) | —(CH₂)₂—Ar | —(CH₂)₃—CH₃ |
| (15′) | —(CH₂)₄—CH₃ | —(CH₂)₂—CH₃ |
| (16′) | —(CH₂)₅—CH₃ | —(CH₂)₃—CH₃ |
| (17′) | —(CH₂)₆—CH₃ | —(CH₂)₄—CH₃ |
| (18′) | —(CH₂)₈—CH₃ | —(CH₂) [symbol] —CH₃ |
| (19′) | —(CH₂)₃—CH(CH₃)CH₃ | —CH₂—CH(CH₃)CH₃ |
| (20′) | —(CH₂)₆—CH═CH₂ | —(CH₂)₅—CH₃ |
| (21′) | —(CH₂)₂—CH═C(CH₃)CH₃ | —CH₂—CH(CH₃)CH₃ |
| (22′) | —(CH₂)₄—CH₃ | —(CH₂)₂—CH₃ |
| (23′) | —(CH₂_{⁾}₅—CH₃ | —(CH₂)₃—CH₃ |
| (24′) | —(CH₂)₆—CH₃ | —(CH₂) [symbol] —CH₃ |
| (2a′) | —(CH₂)₈—(CH₂)₇—CH₃ | (CH₂)₅—(CH₃) |

[symbol]

indicates data missing or illegible when filed

M, n, Hal, t, Ar, Ar′, Cycl, Fur and Sua are as defined previously for compounds (1) to (14).

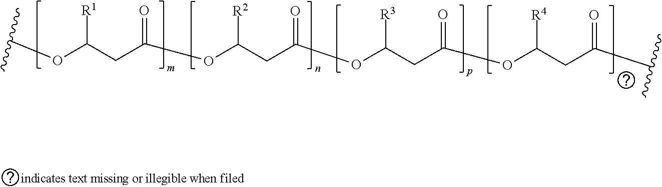

| Compounds | R¹ | R² | R³ | R⁴ |
|---|---|---|---|---|
| (25) | —(CH₂)₈—CH₃ | —(CH₂)₈—CH₃ | —(CH₂)₄—CH₃ | —(CH₂)₂—CH₃ |
| (26) | —(CH₂)₈—CH₃ | —(CH₂)₄—CH₂ | —(CH₂)₂—CH₃ | —CH₃ |
| (27) | —(CH₂)₃—CN | —(CH₂)₃—CH₂ | —(CH₂)₃—CH₃ | —CN |
| (28) | —(CH₂)₂—Ar | —(CH₂)₈—CH₃ | —(CH₃)₂—CH₃ | —Ar |

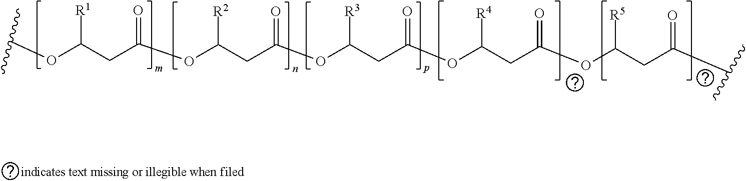

| Com- | | | | | |
|---|---|---|---|---|---|
| pounds | R¹ | R² | R³ | R⁴ | R⁵ |
| (29) | —(CH₂) [symbol] —CH═CH₂ | —(CH [symbol] ) [symbol] —CH₃ | —(CH₂)₃—CH₃ | —(CH₂) [symbol] —CH═CH₂ | —(CH₂) [symbol] —CH═CH₂ |
| (30) | —(CH₂) [symbol] —CH═CH₂ | —(CH₂) [symbol] —CH₃ | —(CH₂)₂—CH₃ | —(CH₂)₇—CH═CH₂ | —(CH₂) [symbol] —CH═CH₂ |
| (31) | —(CH₂)₈—CH₃ | —(CH₂) [symbol] —CH₃ | —(CH₂) [symbol] —CH₃ | —(CH₃) [symbol] —CH₃ | —CH₃ |
| (32) | —(CH₂) [symbol] —CH₃ | —(CH₂)₈—CH₃ | —(CH₂) [symbol] —CH [symbol] | —(CH₂)₄—CH₃ | —(CH₂) [symbol] —CH₃ |
| (33) | —(CH₂)₈—S—CH | —(CH₂)₈—CH₃ | —(CH₂)₂—CH₃ | —(CH₂)₈—S—CH | —(CH₂)₈—S—CH |
| | (CH₃)—C(O)—OH | | | (CH₃)—C(O)—OH | (CH₃)—C(O)—OH |
| (34) | —(CH₂)₈—S— | —(CH₂)₄—CH₃ | —(CH₂)₂—CH₃ | —(CH₂)₈—S— | —(CH₂)₄—S— |
| | (CH₂)₇—CH₃ | | | (CH₃)₇—CH₃ | (CH₂)₇—CH₃ |
| (35) | —(CH₂)₈—S— | —(CH₂)₄—CH₃ | —(CH₃)₂—CH₃ | —(CH₂)₈—S— | —(CH₂)₄—S— |
| | (CH₂)₈—OH | | | (CH₂)₈—OH | (CH₂)₈—OH |
| (36) | —(CH₂)₈—S— | —(CH₂)₄—CH₃ | —(CH₂)₂—CH₃ | —(CH₂) [symbol] —S— | —(CH₂)₄—S— |
| | (CH₂)₂—NH₂ | | | (CH₂)₂—NH₂ | (CH₂)₂—NH₂ |
| (37) | —(CH₂)₈—S-Cycl | —(CH₂)₄—CH₃ | —(CH₂)₂—CH₃ | —(CH₂)₈—S-Cycl | —(CH₂)₄—S—Cycl |
| (38) | —(CH₂)₈—S—CH₂- | —(CH₂)₈—CH₃ | —(CH₂)₂—CH₃ | —(CH₂)₆—S—CH₂-Fur | —(CH₂)₄—S—CH₂-Fur |
| | Fur | | | | |
| (39) | —(CH₂)₈—S-Sug | —(CH₂)₄—CH₃ | —(CH₂)₂—CH₃ | —(CH₂)₆—S-Sug | —(CH₂)₄—S-Sug |
| (40) | —(CH₂)₈—S—(CH₂)₂—Ar | —(CH₂)₄—CH₃ | —(CH₂)₂—CH₃ | —(CH₂)₆—S—(CH₂)₂—Ar | —(CH₂)₄S—(CH₂)₂—Ar |
| (41) | —(CH₂)₈—S—CH₂-Ar′ | —(CH₂)₄—CH₃ | —(CH₂)₂—CH₃ | —(CH₂)₆—S—CH₂—Ar′ | —(CH₂)₄—S—CH₂—Ar′ |
| (42) | —(CH₂)₈—S—CH | —(CH₂)₆—CH₃ | —(CH₂)₃—CH₃ | —(CH₂)₆—S—CH | —(CH₂)₄—S—CH |
| | (CH₃)—C(O)—OH | | | (CH₃)—C(O)—OH\| | (CH₃)—C(O)—OH |
| (43) | —(CH₂)₆-Hal | —(CH₂)₆—CH₃ | —(CH₂)₃—CH₃ | —(CH₂)₃-Hal | —(CH₂)-Hal |
| (44) | 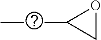 | —(CH₂)₈—CH₃ | —(CH₂)₃ —CH₃ | 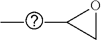 | 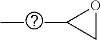 |
| (34a) | —(CH₂)₈—S— | (CH₂)₅—(CH₃) | (CH₂)₃—(CH₃) | —(CH₂)₆ —S— | —(CH₂)₄—S— |
| | (CH₂)₇—CH₃ | | | (CH₂)₇—CH₃ | (CH₂)₇—CH₃ |

[symbol]

indicates data missing or illegible when filed

Preferably, the PHA(s) of the invention are chosen from compounds (2a) (10) (15), (16), (17) and (20). More preferably, the PHA(s) of the invention are chosen from compounds (15), (16) and (17), notably (16).

More preferably, the PHA(s) of the invention are chosen from compounds (2a′), (10′), (15′), (16′) and (17′). More particularly, the PHA(s) of the invention are chosen from compounds (15′), (16′) and (17′), notably (16′).

More particularly, the PHA a) of the invention is compound (23′).

Preferably, the PHA(s) a) of the invention are chosen from compounds (25), (26), (31), (32), and (34a), more preferably, the PHA(s) a) of the invention are chosen from compounds (25), (26), (31) and (32), notably (26).

The PHA copolymer(s) of the invention preferably have a number-average molecular weight ranging from 50 000 to 150 000.

The molecular weight may notably be measured by size exclusion chromatography. A method is described below in the examples.

The PHA copolymer(s) are particularly present in composition C1 according to the invention in a content ranging from 0.1% to 65% by weight and preferably ranging from 0.1% to 60% by weight relative to the total weight of the composition; particularly 1% to 50% by weight and more particularly ranging from 3% to 40% by weight and preferably ranging from 5% to 35% by weight, more preferably ranging from 10% to 30%, and better ranging from 15% to 20% by weight relative to the total weight of the composition by weight relative to the total weight of the composition.

### Method for Preparing the PHA Copolymer(s):

The methods for preparing the PHA copolymer(s) of the invention are known to those skilled in the art. Mention may notably be made of the use of “functionalizable” PHA-producing microbial strains.

The term “functionalizable” means that the PHA copolymer(s) comprise a hydrocarbon-based chain comprising one or more atoms or groups that are capable of reacting chemically with another reagent—also referred to as “reactive atoms or reactive groups”—to give a Σ covalent bond with said reagent. The reagent is, for example, a compound comprising at least one nucleophilic group and said functionalized hydrocarbon-based chain comprises at least one electrophilic or nucleofugal atom or group, the nucleophilic group(s) reacting with the electrophilic group(s) to covalently graft Σ the reagent. The nucleophilic reagent may also react with one or more unsaturations of the alkenyl group(s) to also lead to grafting by covalent bonding of the functionalized hydrocarbon-based chain with said reagent. The addition reaction may also be radical-based, an addition of Markovnikov or anti-Markovnikov type, or nucleophilic or electrophilic substitution. The addition or condensation reactions may or may not take place via a radical route, with or without the use of catalysts or of enzymes, with heating preferably to a temperature less than or equal to 100° C. or without supplying heat, under a pressure of greater than 1 atm or otherwise, under an inert atmosphere or otherwise, or under oxygen or otherwise.

The term “nucleophilic” refers to any atom or group which is electron-donating by an inductive effect +I and/or a mesomeric effect +M. Electron-donating groups that may be mentioned include hydroxyl, thiol and amino groups.

The term “electrophilic” refers to any atom or group which is electron-withdrawing by an inductive effect −1 and/or a mesomeric effect −M. Electron-withdrawing species that may be mentioned include.

The microorganisms producing PHAs of the invention notably bearing a hydrocarbon-based chain may be naturally produced by the bacterial kingdom, such as Cyanobacteria of the order of Nostocales (e.g.: Nostoc muscorum, Synechocystis and Synechococcus) but mainly by the Proteobacteria, for example in the class of:
beta-Proteobacteria, of the order Burkholderiales (Cupriavidus negator synonym Ralstonia eutropha)
alpha-Proteobacteria, of the order Rhodobacteriales (Rhodobacter capsulatus marine and photosynthetic)
gamma-Proteobacteria, of the order Pseudomonales of the family Moraxellaceae (Acinetobacter juni).

Among the microorganisms of the bacterial kingdom, the genera Azotobacter, Hydrogenomomas or Chromatium are the most representative of the PHA-producing organisms.

The organisms which naturally produce PHAs bearing notably a C₃-C₅ hydrocarbon-based chain are notably Proteobacteria, such as gamma-Proteobacteria, and more particularly of the order Pseudomonales of the family Pseudomonas such as Pseudomonas resinovorans, Pseudomonas putida, Pseudomonas fluorescens, Pseudomonas aeruginosa, Pseudomonas citronellolis, Pseudomonas mendocina, Pseudomonas chlororaphis and preferably Pseudomonas putida GPo1 and Pseudomonas putida KT2440, preferably Pseudomonas putida and in particular Pseudomonas putida GPo1 and Pseudomonas putida KT2440.

Certain organisms may also naturally produce PHAs without belonging to the order of Pseudomonales, such as Commamonas testosteroni which belongs to the class of beta-Proteobacteria of the order Burkholderiales of the family of Comamonadaceae.

The microorganism producing PHAs according to the invention may also be a recombinant strain if a 3-oxidation PHA synthase metabolic pathway is present. The 3-oxidation PHA synthase metabolic pathway is mainly represented by four classes of enzymes, EC: 2.3.1 B2, EC: 2.3.1 B3, EC: 2.3.1 B4 and EC: 2.3.1 B5.

The recombinant strain may be from the Bacteria kingdom, for instance Escherichia coli, or from the Plantae kingdom, for instance Chlorella pyrenoidosa (International Journal of Biological Macromolecules, 116, 552-562 “Influence of nitrogen on growth, biomass composition, production, and properties of polyhydroxyalkanoates (PHAs) by microalgae”) or from the Fungi kingdom, for instance Saccharomyces cerevisiae or Yarrowia lipolytica: Applied Microbiology and Biotechnology 91, 1327-1340 (2011) “Engineering polyhydroxyalkanoate content and monomer composition in the oleaginous yeast Yarrowia lipolytica by modifying the β-oxidation multifunctional protein”).

Use may also be made of genetically modified microorganisms, which may make it possible, for example, to increase the production of PHA, and/or to increase the oxygen consumption capacity, and/or to reduce the autolysis and/or to modify the monomer ratio.

It is known that, for PHAs, a large portion of the total production cost is devoted to the culture medium and mainly to the substrate/carbon source. Use may thus be made of genetically modified microorganisms using a smaller amount of nutrient (carbon source) for their growth, for example microorganisms that are photo-autotrophic by nature, i.e. using light and CO₂ as main energy source.

The copolymer may be obtained in a known manner by biosynthesis, for example with the microorganisms belonging to the genus Pseudomonas, such as Pseudomonas resinovorans, Pseudomomonas putida, Pseudomonas fluorescens, Pseudomonas aeruginosa, Pseudomonas citronellolis, Pseudomonas mendocina, Pseudomonas chlororaphis and preferably Pseudomonas putida; and with a carbon source which may be a C₂-C₂₀, preferably C₆-C₁₈, carboxylic acid, such as acetic acid, propionic acid, butyric acid, hexanoic acid, heptanoic acid, octanoic acid, nonanoic acid, dodecanoic acid, or an alkenoic acid such as undecylenic acid; a saccharide, such as fructose, maltose, lactose, xylose, arabinose, etc.); an n-alkane, such as hexane, octane or dodecane; an n-alcohol, such as methanol, ethanol, octanol or glycerol; methane or carbon dioxide.

The biosynthesis may optionally be performed in the presence of an inhibitor of the β-oxidation pathway, such as acrylic acid, methacrylic acid, propionic acid, cinnamic acid, salicylic acid, pentenoic acid, 2-butynoic acid, 2-octynoic acid or phenylpropionic acid, and preferably acrylic acid.

According to one embodiment, the process for preparing the PHAs of the invention uses microbial cells which produce PHAs via genetically modified microorganisms (GMOs). The genetic modification may increase the production of PHA, increase the oxygen absorption capacity, increase the resistance to the toxicity of solvents, reduce the autolysis, modify the ratio of the PHA comonomers, and/or any combination thereof. In some of these embodiments, the modification of the comonomer ratio of the unit (A) increases the amount of predominant monomer versus (B) of the PHA of the invention which is obtained. In another embodiment, the PHA-producing microbial cells reproduce naturally.

By way of example, a genetically modified microbial strain producing PHA that is functionalizable or comprising a reactive group that may be mentioned is Pseudomonas entomophila LAC23 (Biomacromolecules. 2014 Jun. 9; 15(6):2310-9. doi: 10.1021/bm500669s).

It is also possible to use genetically modified microorganisms which produce phenylvaleric-co-3-hydroxydodecanoic copolymers (Sci. China Life Sci., Shen R., et al., 57, No. 1, (2014) with a strain such as Pseudomonas entomophila LAC23.

Nutrients, such as water-soluble salts based on nitrogen, phosphorus, sulfur, magnesium, sodium, potassium and iron, may also be used for the biosynthesis.

The appropriate known conditions of temperature, pH and dissolved oxygen (O_{D}) can be used for the culturing of the microorganisms.

The microorganisms may be cultured according to any known method of culturing, such as in a bioreactor in continuous or batch mode, in fed or unfed mode.

The biosynthesis of the polymers used according to the invention is notably described in the article “Biosynthesis and Properties of Medium-Chain-Length Polyhydroxyalkanoates with Enriched Content of the Dominant Monomer”, Xun Juan et al., Biomacromolecules 2012, 13, 2926-2932, and in patent application WO 2011/069244.

The microbial strains producing PHA which is functionalizable or comprising a reactive group, as defined previously, are, for example, of the genus Pseudomonas such as P. cichorii YN2, P. citronellolis, P. jessenii, and more generally with species of Pseudomonas putida such as Pseudomonas putida GPo1 (synonym of Pseudomonas oleovorans), P. putida KT2442, P. putida KT2440, P. putida KCTC 2407 and P. putida BM01, and in particular P. putida KT2440.

### The Carbon Source(s):

One means for gaining access to the PHAs of the invention is to introduce one or more organic compounds into the culture medium, this or these organic compounds representing one or more carbon sources preferably chosen from alkanes, alkenes, alcohols, carboxylic acids and a mixture thereof.

In one embodiment, the organic compound(s) will preferably be chosen from alcohols, carboxylic acids and a mixture thereof.

The carbon source(s) may be classified in two categories:
1) Carbon source via one or more organic compounds introduced into the medium:

According to a particular embodiment of the invention, the organic compound(s) are chosen from alcohols, in particular (C₅-C₂₀)alkanols, and/or carboxylic acids, in particular optionally substituted and/or interrupted (C₅-C₂₀)alkanoic acids, notably (C₅-C₂₀)alkanoic acids such as (C₇-C₁₁)alkanoic acids, for instance nonanoic acid or pelargonic acid and/or (C₅-C₂₀)alkenoic acids, notably (C₅-C₂₀)alkenoic acids such as (C₇-C₁₁)alkenoic acids, for instance undecylenic acid, and mixtures thereof.

The carbon source(s) may be classified into three groups according to their intended use:
group A: the organic compound may aid the growth of the productive strain and aids the production of PHA structural linked to the organic compound.
group B: the organic compound may aid the growth of the strain but does not participate in the production of PHA structurally linked to the organic compound.
group C: the organic compound does not participate in the growth of the strain.

Such microbiological processes are known to those skilled in the art, notably in the scientific literature. Mention may be made of: International Journal of Biological Macromolecules 28, 23-29 (2000); The Journal of Microbiology, 45, No. 2, 87-97, (2007).

According to one variant, the integration of the substrate that is structurally linked to the reactive atom(s) or to the reactive group(s) of the PHA(s) of the invention is introduced directly into the medium as sole carbon source in a medium suitable for microbial growth. (Example: group A for P. putida GPo1: alkenoic acid, notably terminal).

According to another variant, the integration of the substrate that is structurally linked to the reactive atom(s), notably halogen, or to the reactive group(s) of the PHA(s) of the invention is introduced into the medium as carbon source with a second carbon source as co-substrate which is also structurally linked to the PHA, in a medium suitable for microbial growth. (Example: group B for P. putida GPo1: haloalkanoic acids which are preferably terminal, such as terminal bromoalkanoic acids).

According to yet another variant, the integration of the substrate that is structurally linked to the reactive atom(s), notably halogen, or to the reactive group(s) of the PHA(s) of the invention may be introduced directly into the medium as carbon source with a second carbon source as co-substrate which is also structurally linked to the PHA(s) and a third carbon source as co-substrate which is not structurally linked to the PHA(s), in a medium suitable for microbial growth. (Example: group C glucose or sucrose).

In one embodiment, the β-oxidation pathway inhibitor is acrylic acid, 2-butynoic acid, 2-octynoic acid, phenylpropionic acid, propionic acid, trans-cinnamic acid, salicylic acid, methacrylic acid, 4-pentenoic acid or 3-mercaptopropionic acid.

In one embodiment of the first aspect, the functionalized fatty acid is a functionalized hexanoic acid, functionalized heptanoic acid, functionalized octanoic acid, functionalized nonanoic acid, functionalized decanoic acid, functionalized undecanoic acid, functionalized dodecanoic acid or functionalized tetradecanoic acid.

The functionalization may be introduced by means of an organic compound chosen from precursors of the alcohol and/or carboxylic acid category, notably:
for functionalization of the PHA(s) with a branched alkyl group: see, for example, Applied and Environmental Microbiology, 60, No. 9, 3245-325 (1994);
for functionalization of the PHA(s) with a linear alkyl group comprising a terminal cyclohexyl unit: see, for example, doi.org/10.1016/S0141-8130(01)00144-1;
for functionalization of the PHA(s) with an unsaturated alkyl group which is preferably terminal: see, for example, doi.org/10.1021/bm8005616);
for functionalization of the PHA(s) with a linear alkyl group comprising a halogen preferably at the end of the hydrocarbon-based chain (doi.org/10.1021/ma00033a002);
for functionalization of the PHA(s) with a (hetero)aromatic alkyl group, for example phenyl, benzoyl, phenoxy, see, for example, J. Microbiol. Biotechnol., 11, 3, 435-442 (2001);
for functionalization of the PHA(s) with a linear alkyl group comprising a heteroatom notably at the end of the hydrocarbon-based chain, see, for example, DOI 10.1007/s00253-011-3099-4;
for functionalization of the PHA(s) with a linear alkyl group comprising a cyano function notably at the end of the hydrocarbon-based chain, see, for example, doi.org/10.1111/j.1574-6968.1992.tb05839.x;
for functionalization of the PHA(s) with a linear alkyl group comprising an epoxy function notably at the end of the hydrocarbon-based chain, see, for example, doi.org/10.1016/S1381-5148(97)00024-2.

The review International Microbiology 16:1-15 (2013) doi:10.2436/20.1501.01.175 also mentions the majority of the functionalized native PHAs.

In a particular embodiment of the invention, the fatty acid from group A is chosen from 11-undecenoic acid, 10-epoxyundecanoic acid, 5-phenylvaleric acid, citronellol and 5-cyanopentanoic acid.

In a particular embodiment of the invention, the fatty acid from group A is chosen from halooctanoic acids such as 8-bromooctanoic acid.

In a particular embodiment of the invention, the carbon source from group C is a monosaccharide, preferably glucose.
2) Carbon source in the presence of oxidation inhibitor introduced into the medium:

Another aspect of the invention is the use of the PHA-producing microbial strains in a medium that is suitable for microbial growth, said medium comprising: a substrate which is structurally linked to the PHA(s); at least one carbon source which is not structurally linked to the PHA(s); and at least one oxidation and notably β-oxidation pathway inhibitor. This allows the growth of the microbial cells to take place in said medium, the microbial cells synthesizing the PHA polymer(s) of the invention; preferably copolymer particularly containing more than 95% of identical units, which has a comonomer ratio of unit (A) and of unit (B) which differs from that obtained in the absence of the β-oxidation pathway inhibitor.

The scheme below illustrates, by way of example, the functionalization of PHA copolymers according to the invention starting from a PHA copolymer bearing an unsaturated hydrocarbon-based chain, according to Scheme 1 below:

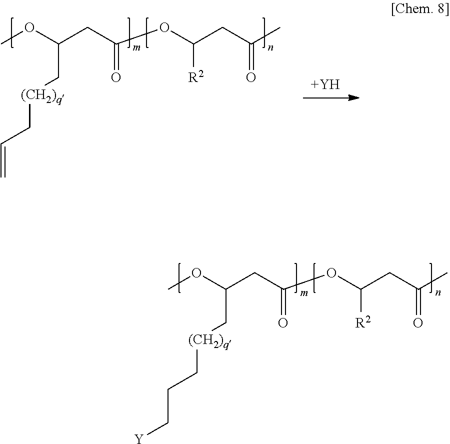

in which Scheme 1:
   R², m and n are as defined previously;
   Y represents a group chosen from Hal such as chlorine or bromine, hydroxyl, thiol, (di)(C₁-C₄)(alkyl)amino, R—X with R representing a group chosen from α) cycloalkyl such as cyclohexyl, β) heterocycloalkyl such as a sugar, preferably a monosaccharide such as glucose, γ) (hetero)aryl such as phenyl; δ) a cosmetic active agent as defined previously; ε) (C₁-C₂₀)alkyl, (C₂-C₂₀)alkenyl, (C₂-C₂₀)alkynyl; and X representing a′) O, S, N(Rₐ) or Si(R_{b})(R_{c}) or e) linear or branched (C₁-C₂₀)alkyl, with Rₐ, R_{b} and Rₑ as defined previously;
   q′ represents an integer inclusively between 2 and 20, preferably between 3 and 10, more preferentially between 4 and 8 such as 6, better still between 3 and 8, preferably between 4 and 6, such as 5.

Other reactions may be performed using double or triple unsaturations such as Michael or Diels-Alder additions, radical reactions, catalytic (notably with Pd or Ni) or non-catalytic hydrogenation reactions, halogenation reactions, notably with bromine, hydration reactions or oxidation reactions, which may or may not be controlled, and reactions on electrophiles as represented schematically below.

According to a particular embodiment of the invention, the PHA copolymers comprise
a linear or branched, saturated hydrocarbon-based chain R¹, substituted and/or interrupted with groups as defined previously for R¹, comprising in total between 5 and 30 carbon atoms, preferably between 6 and 20 carbon atoms and more particularly between 7 and 11 carbon atoms, and
a hydrocarbon-based chain R² representing a linear or branched (C₃-C₂₀)alkenyl, particularly (C₅-C₁₄)alkenyl and more particularly (C₇-C₁₀)alkenyl radical, which is preferably linear and comprising only one unsaturation at the chain end, in particular —[CR⁴(R⁵)]_{q}—C(R⁶)═C(R⁷)—R⁸ with R⁴, R⁵, R⁶, R⁷ and R⁸, which may be identical or different, representing a hydrogen atom or a (C₁-C₄)alkyl group such as methyl, preferably a hydrogen atom, and q represents an integer inclusively between 2 and 20, preferably between 3 and 10, more preferentially between 4 and 8 such as 6, such as —[CH₂]_{q}—CH═CH₂ and q represents an integer inclusively between 3 and 8, preferably between 4 and 6, such as 5,
said chain R² comprising between 1% and 99%, preferentially between 2% and 50% and even more preferentially between 3% and 40% of unsaturations, and even more particularly between 3% and 30% of unsaturations, better still between 5% and 20% of unsaturations. According to this particular embodiment of the invention in which the PHA copolymers comprise unsaturations, these unsaturations may be chemically modified:
A) via addition reactions, such as radical additions, Michael additions, electrophilic additions, Diels-Alder, halogenation, hydration or hydrogenation reaction, and preferably hydrothiolation reaction with particles, chemical compounds or polymers.

In particular, the hydrothiolation reactions may be performed in the presence of a thermal initiator, a redox initiator or a photochemical initiator and of an organic compound bearing a sulfhydryl group, notably chosen from:
linear, branched, cyclic or aromatic alkanethiols including 1 to 14 carbon atoms, such as methane-, ethane-, propane-, pentane-, cyclopentane-, hexane-, cyclohexane-, heptane-, octane-, phenylethane-, 4-tert-butylphenylmethane- or 2-furanmethane-thiol, preferably hexane-, cyclohexane-, heptane-, octane-, phenylethane-, 4-tert-butylphenylmethane- or 2-furanmethane-thiol;
organosiloxanes bearing a thiol function, such as (3-mercaptopropyl)trimethoxysilane, (3-mercaptopropyl)methyldimethoxysilane, 2-(triethoxysilyl)ethanethiol or mercaptopropyl-isobutyl-POSS;
thiolated silicone oils, notably those described in the document DOI: 10.1016/j.actbio.2015.01.020);
thiolated oligomers or polymers bearing a reactive function, such as an amine, an alcohol, an acid, a halogen, a thiol, an epoxide, a nitrile, an isocyanate, a heteroatom, preferably cysteine, cysteamine, N-acetylcysteamine, 2-mercaptoethanol, 1-mercapto-2-propanol, 8-mercapto-1-octanol, thiolactic acid, thioglycolic acid, 3-mercaptopropionic acid, 11-mercaptoundecanoic acid, polyethylene glycol dithiol, 3-mercaptopropionitrile, 1,3-propanedithiol, 4-cyano-1-butanethiol, 3-chloro-1-propanethiol, 1-thio-β-D-glucose tetraacetate; and
thiols which may be obtained from disulfide reduction, such as phenyl disulfide or furfuryl disulfide.

Examples of initiators that may be mentioned include: tert-butyl peroxy-2-ethylhexanoate, cumene perpivalate, tert-butyl peroxylaurate, benzoyl peroxide, lauroyl peroxide, octanoyl peroxide, di-tert-butyl peroxide, tert-butylcumyl peroxide, dicumyl peroxide, 2,2′-azobisisobutyronitrile, 2,2′-azobis(2-methylbutyronitrile), 2,2′-azobis(2,4-dimethylvaleronitrile), 2,2′-azobis(4-methoxy-2,4-dimethylvaleronitrile), 1,1-bis(tert-butylperoxy)-3,3,5-trimethylcyclohexane, 1,1-bis(tert-butylperoxy)cyclohexane, 1,4-bis(tert-butylperoxycarbonyl)cyclohexane, 2,2-bis(tert-butylperoxy)octane, n-butyl 4,4-bis(tert-butylperoxy)valerate, 2,2-bis(tert-butylperoxy)butane, 1,3-bis(tert-butylperoxyisopropyl)benzene, 2,5-dimethyl-2,5-bis(tert-butylperoxy)hexane, 2,5-dimethyl-2,5-bis(benzoylperoxy)hexane, di-tert-butyl diperoxyisophthalate, 2,2-bis(4,4-di-tert-butylperoxycyclohexyl)propane, di-tert-butyl peroxy-α-methylsuccinate, di-tert-butyl peroxydimethylglutarate, di-tert-butyl peroxyhexahydroterephthalate, di-tert-butyl peroxyazelate, 2,5-dimethyl-2,5-bis(tert-butylperoxy)hexane, diethylene glycol bis(tert-butylperoxycarbonate), di-tert-butyl peroxytrimethyladipate, tris(tert-butylperoxy)triazine, vinyltris(tert-butylperoxy)silane phenothiazine, tetracene, perylene, anthracene, 9,10-diphenylanthracene, thioxanthone, benzophenone, acetophenone, xanthone, fluorenone, anthraquinone, 9,10-dimethylanthracene, 2-ethyl-9,10-dimethyloxyanthracene, 2,6-dimethylnaphthalene, 2,5-diphenyl-1,3,4-oxadiazole, xanthopinacol, 1,2-benzanthracene, 9-nitroanthracene. Each of these initiators may be used alone or in combination with others.

The chemical reactions mentioned previously are known to those skilled in the art. Mention may notably be made of the following documents: Synthesis and preparation of PHAs modified with polyethylene glycol dithiol: 10.1021/acs.biomac.9b00479; Biomacromolecules, 19, 3536-3548 (2018); Synthesis and preparation of PHAs modified with mercaptohexanol: 10.1021/acs.biomac.8b01257; Biomacromolecules, 20, 2, 645-652 (2019); Synthesis and preparation of PHAs modified with hydroxycinnamic acid sulfate, and zosteric acid: 10.1021/bm049962e; Biomacromolecules, 5, 4, 1452-1456 (2004); Radical addition of methyl methacrylate to a PHOUn: 10.1002/1521-3935(20010701)202:11<2281::AID-MACP2281>3.0.CO; 2-9; Macromolecular Chemistry and Physics, vol. 202, 11, 2281-2286 (2001); Synthesis and preparation of PHAs modified with a polysilsesquioxane (POSS): 10.1016/j.polymer.2005.04.020; Polymer Vol. 46, 14, 5025-5031 (2005); Grafting of thio-beta-glucose onto unsaturated side chains: 1022-1336/99/0202-0091$17.50+0.50/0; Macromol. Rapid Commun., 20, 91-94 (1999);
and/or
B) via oxidation reactions, which may or may not be controlled, for example with permanganates of a concentrated or dilute alkaline agent, or ozonolysis, oxidation in the presence of a reducing agent, making it possible to obtain novel materials bearing hydroxyl, epoxide or carboxyl groups in the terminal position of the side chains.

The chemical reactions mentioned previously are known to those skilled in the art. Mention may notably be made of the following documents: 10.1021/bm049337; Biomacromolecules, vol. 6, 2, 891-896 (2005); 10.1016/S0032-3861(99)00347-X; Polymer, vol. 41, 5, 1703-1709 (2000); 10.1021/ma9714528 and 10.1016/S1381-5148(97)00024-2; Macromolecules, 23, 15, 3705-3707 (1990); 10.1016/S0032-3861(01)00692-9; Polymer, vol. 43, 4, 1095-1101 (2002); 10.1016/S0032-3861(99)00347-X; Polymer, vol. 41, 5, 1703-1709 (2000); and 10.1021/bm025728h; Biomacromolecules, vol. 4, 2, 193-195 (2003).

Example of functionalization of PHA copolymers according to the invention starting from a PHA copolymer bearing a hydrocarbon-based chain containing an epoxide group, according to Scheme 2 below:

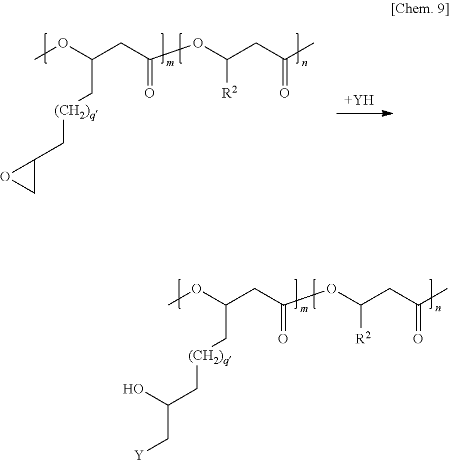

in which Scheme 2 Y, m, n, q′ and R² are as defined in Scheme 1.

The epoxide structure may be obtained via a conventional method known to those skilled in the art, whether via biotechnological processes or via chemical processes such as oxidation of unsaturation as mentioned previously. The peroxide group(s) may react with carboxylic acids, maleic anhydrides, amines, alcohols, thiols or isocyanates, all these reagents including at least one linear or branched, cyclic or acyclic, saturated or unsaturated C₁-C₂₀ hydrocarbon-based chain, or borne by an oligomer or polymer, in particular amino (poly)saccharides such as compounds derived from chitosan and (poly)sil(ox)anes; 3-glycidyloxypropyltrimethoxysilane, 3-aminopropyltriethoxysilane 3-(trimethoxysilyl)propylcarbamic acid, diethanolamine, or 3-mercapto-1-propanesulfonate of alkali metal or alkaline-earth metal salts such as sodium. The epoxide groups may also react with water.

Mention may notably be made of the following documents:
Preparation of PHA bearing charges starting with diethanolamine: 10.1021/bm8005616, Biomacromolecules, vol. 9, 8 2091-2096 (2008);
Preparation of PHA bearing charges starting with sodium 3-mercapto-1-propanesulfonate: 10.1021/acs.biomac.9b00870 Biomacromolecules, vol. 20, 9, 3324-3332 (2019);
Preparation of PHA including a native epoxide unit: 10.1016/S1381-5148(97)00024-2); Reactive and Functional Polymers, vol. 34, 1, 65-77 (1997).

Example of functionalization of PHA copolymers according to the invention starting from a PHA copolymer bearing a hydrocarbon-based chain containing a nucleofugal group, according to Scheme 3 below:

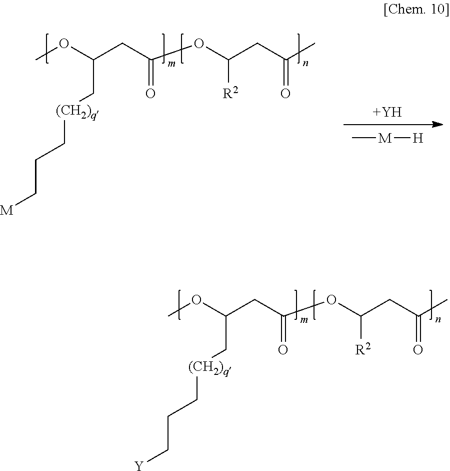

in which Scheme 3 Y, m, n, q′ and R² are as defined in Scheme 1. M corresponds to an organic or inorganic nucleofugal group, which may be substituted with a nucleophilic group; preferably, said nucleophile is a heteroatom which is electron-donating via the +I and/or +M effect such as O, S or N. Preferably, the nucleofugal group M is chosen from halogen atoms such as Br, and mesylate, tosylate or triflate groups. This is a reaction known to those skilled in the art. Mention may be made, for example, of the following document: 10.1016/j.ijbiomac.2016.11.118, International Journal of Biological Macromolecules, vol. 95, 796-808 (2017).

Example of functionalization of PHA copolymers according to the invention starting from a PHA copolymer bearing a hydrocarbon-based chain containing a cyano group, according to Scheme 4 below:

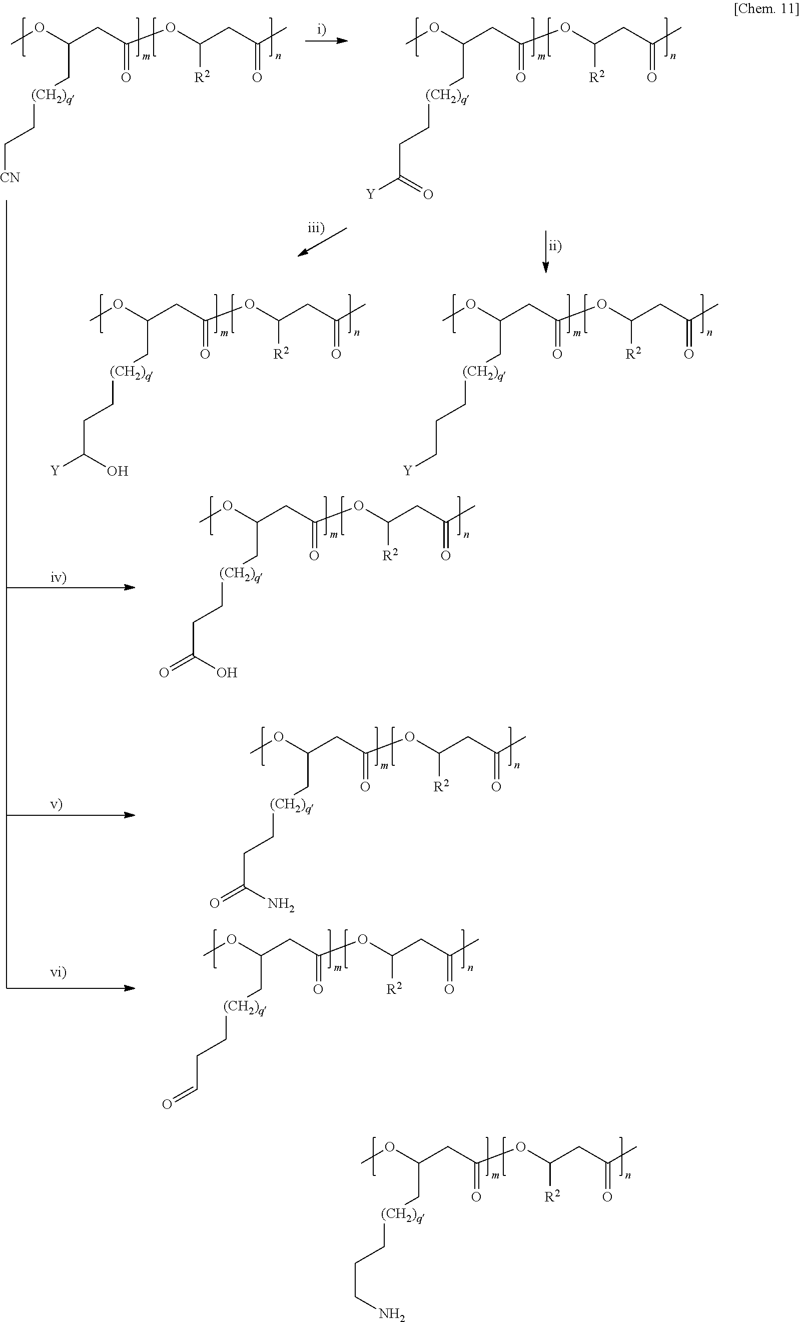

in which Scheme 4 Y, m, n, q′ and R² are as defined in Scheme 1.

In a first step i), the PHA copolymer bearing a side chain containing a cyano or nitrile group reacts with an organo-alkali metal or organomagnesium compound Y—MgHal, Y—Li or Y—Na, followed by hydrolysis to give the PHA copolymer bearing a side chain containing a group Y grafted with a ketone function. The ketone function may be converted into a thio ketone by thionation, for example with S8 in the presence of amine, or with Lawesson's reagent. Said thio ketone, after total reduction ii) (for example by Clemmensen reduction), leads to the PHA copolymer bearing a side chain containing a group Y grafted with an alkylene group. Alternatively, said thio ketone may undergo a controlled reduction iii) with a conventional reducing agent to give the PHA copolymer bearing a side chain containing a group Y grafted with a hydroxyalkylene group. The cyano group of the starting PHA copolymer can react with water after hydration v) to give the amide derivative, after hydrolysis iv) to the carboxyl derivative. The cyano group of the starting PHA copolymer can also, after reduction vi), give the amine derivative or the ketone derivative. PHA copolymers with a hydrocarbon-based chain bearing a nitrile function are prepared via conventional methods known to those skilled in the art. Mention may be made, for example, of the document: 10.1016/0378-1097(92)90311-B, FEMS Microbiology Letters, vol. 103, 2-4, 207-214 (1992).

Example of functionalization of PHA copolymers according to the invention starting from a PHA copolymer bearing a hydrocarbon-based chain at the chain end, according to Scheme 5 below:

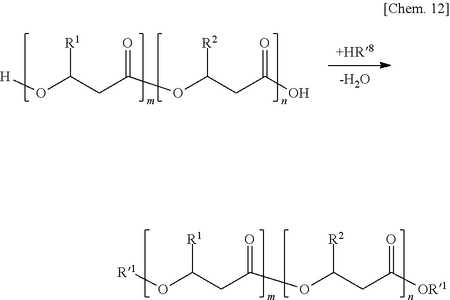

in which Scheme 5 R¹, R², m, n and Y are as defined previously, and R′¹ represents a hydrocarbon-based chain chosen from i) linear or branched (C₁-C₂₀)alkyl, ii) linear or branched (C₂-C₂₀)alkenyl, iii) linear or branched (C₂-C₂₀)alkynyl; preferably, the hydrocarbon-based group is linear; said hydrocarbon-based chain being substituted with one or more atoms or groups chosen from: a) halogens such as chlorine or bromine, b) hydroxyl, c) thiol, d) (di)(C₁-C₄)(alkyl)amino, e) (thio)carboxyl, f) (thio)carboxamide —C(O)—N(Rₐ)₂ or —C(S)—N(Rₐ)₂, f) cyano, g) iso(thio)cyanate, h) (hetero)aryl such as phenyl or furyl, and i) (hetero)cycloalkyl such as anhydride, or epoxide, j) a cosmetic active agent chosen from coloured or uncoloured, fluorescent or non-fluorescent chromophores such as those derived from optical brighteners, or chromophores derived from UVA and/or UVB screening agents, and anti-ageing active agents.

These chain-end grafts onto PHA polymers are known to those skilled in the art. Mention may be made, for example, of the following documents:
Preparation of PHA oligomers by thermal degradation: 10.1021/bm0156274; Biomacromolecules, vol. 3, 1, 219-224 (2002);
Preparation of PHA oligomers by transesterification: 10.1021/ma011420r, Macromolecules, vol. 35, 3. 684-689 (2002);
Preparation of PHA oligomers by hydrolysis: 10.1016/0032-3861(94)90590-8Polymer vol. 35, 19, 4156-4162 (1994);
Preparation of PHA oligomers by methanolysis: 10.1021/bm060981t, Biomacromolecules, vol. 8, 4, 1255-1265 (2007).

Mention may also be made of other methods known to those skilled in the art:
Synthesis and characterization of PHA grafted with ascorbic acid: 10.1016/j.ijbiomac.2018.11.052; International Journal of Biological Macromolecules, vol. 123: 7 (2019);
Preparation of PHB-b-PHO copolymers by polycondensation with divinyl adipate catalysed with a lipase: 10.1021/bm9011634, Biomacromolecules, vol. 10, 12, 3176-3181 (2009);
Synthesis of PHB-b-PHO copolymers coupled via a diisocyanate junction: 10.1021/ma012223v; Macromolecules, vol. 35, 13, 4946-4950 (2002);
Preparation of PHO oligomers on chitosan by condensation between the carboxylic acid end of the PHO and the amine functions of the chitosan: 10.1002/app.24276; Journal of Applied Polymer Science, vol. 103, 1, (2006);
Transesterification of PHAs with propargyl alcohol in order to produce PHA oligomers that are modifiable by “click” chemistry: 10.1016/j.reactfunctpolym.2011.12.005; Reactive and Functional Polymers, vol. 72, 2, 160-167 (2012);
Preparation of PHO-b-PCL copolymer: 10.1002/mabi.200400104; Macromolecular Bioscience, vol. 4, 11 (2004);
Preparation of PHO-b-PEG copolymer: 10.1002/macp.201000562; Macromolecular Chemistry and Physics; vol. 212, 3, (2010);
Epoxidation of chain-end unsaturation and chain-end grafting of acid: 10.14314/polimery.2017.317; Polimery, vol. 62, 4, 317-322 (2017);
Grafting of organosiloxane unit at chain end onto PHA: 10.1016/j.reactfunctpolym.2014.09.008; Reactive and Functional Polymers, vol. 84, 53-59 (2014).

The combination of grafted PHA copolymers of the invention described previously, according to Scheme 6:

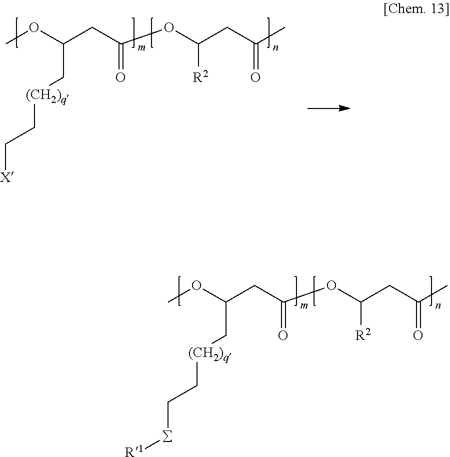

in which Scheme 6 R′¹, R², m, n and Y are as defined previously, and X′ represents a reactive atom or group that is capable of reacting with an electrophilic [symbol] or nucleophilic [symbol] atom or group to create a Σ covalent bond; if X′ is an electrophilic or nucleofugal group, then it can react with a reagent R′¹− [symbol] ; if X′ is a nucleophilic group [symbol] , then it can react with R′¹- [symbol] to create a Σ covalent bond.

By way of example, the Z covalent bonds or bonding group that may be generated are listed in the table below, from condensation of electrophiles with nucleophiles:

TABLE 1

| Electropliles E | Nucleophiles Nu | Covalent bonds Σ |
|---|---|---|
| Activated esters* | Amines | Carboxamides |
| Acyl azides** | Amines | Carboxamides |
| Acyl halides | Amines | Carboxamides |
| Acyl halides | Alcohols | Esters |
| Acyl cyanides | Alcohols | Esters |
| Acyl cyanides | Amines | Carboxamides |
| Alkyl halides | Amines | Alkylamines |
| Alkyl halides | Carboxylic acids | Esters |
| Alkyl halides | Thiols | Thioesters |
| Alkyl halides | Alcohols | Ethers |
| Sulfonic acids and salts thereof | Thiols | Thioethers |
| Sulfonic acids and salts thereof | Carboxylic acids | Esters |
| Sulfonic acids and salts thereof | Alcohols | Ethers |
| Anhydrides | Alcohols | Esters |
| Anhydrides | Amines | Carboxamides |
| Aryl halides | Thiols | Thioethers |
| Aryl halides | Amines | Arylamines |
| Aziridines | Thiols | Thioethers |
| Carboxylic acids | Amines | Carboxamides |
| Carboxylic acids | Alcohols | Esters |
| Carbodiimides | Carboxylic acids | N-acylureas |
| Diazoalkanes | Carboxylic acids | Esters |
| Epoxides | Thiols | Thioethers |
| Haloacetamides | Thiols | Thioethers |
| Imide esters | Amines | Amidines |
| Isocyanates | Amines | Ureas |
| Isocyanates | Alcohols | Urethanes |
| Isothiocyanates | Amines | Thioureas |
| Maleimides | Thiols | Thioethers |
| Sulfonic esters | Amines | Alkylamines |
| Sulfonic esters | Thiols | Thioethers |
| Sulfonic esters | Carboxylic acids | Esters |
| Sulfonic esters | Alcohols | Ethers |
| Sulfonyl halides | Amines | Sulfonamides |

*activated esters of general formula -CO-LG with LG representing a leaving group such as oxysuccinimidyl, oxybenzotriazolyl, optionally substituted aryloxy:
**acyl azides can rearrange to give isocyanates

It is also possible, starting with a PHA functionalized on a side chain, to perform chain-end grafting in a second stage as described in Scheme 7. The reverse is also true,

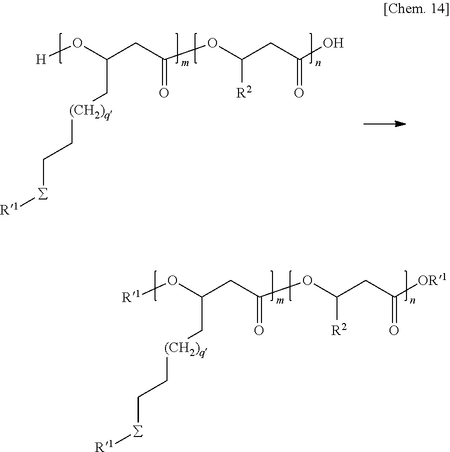

in which the chain-end grafting may be performed in a first stage, followed by performing functionalization of a functionalizable side chain in a second stage.
in which Scheme 7 R′¹, R², m, n and Y are as defined previously, and

All these chemical reactions are known to those skilled in the art. Mention may be made, for example, of the following documents:
Synthesis and preparation of PHAs modified with thiol-ene followed by reaction on the new grafted function: 10.1021/ma0304426; Macromolecules, vol. 37, 2, 385-389 (2004);
Grafting of PEG and of PLA onto PHAs functionalized with acids: 10.1002/marc.200900803 and 10.1002/mabi.200390033;
Synthesis and preparation of PHAs modified with polyethylene glycol dithiol: 10.1021/acs.biomac.9b00479.

### b) the Crystallizable Fatty Substance(s)

Composition C1 or C1′ according to the invention comprises at least one crystallizable fatty substance.

The term “fatty substance” means an organic compound that is insoluble in water at ordinary room temperature (25° C.) and at atmospheric pressure (760 mmHg) (solubility of less than 5%, preferably 1% and even more preferentially 0.1%). They bear in their structure at least one hydrocarbon-based chain including at least 6 carbon atoms or a sequence of at least two siloxane groups. In addition, the fatty substances are generally soluble in organic solvents under the same temperature and pressure conditions, for instance chloroform, ethanol, benzene, liquid petroleum jelly or decamethylcyclopentasiloxane.

The crystallizable fatty substance(s) of the invention are of natural or synthetic origin, preferably natural, more preferentially of plant origin or of animal origin from insects. They are different from fatty acids since salified fatty acids constitute soaps which are generally soluble in aqueous media.

For the purposes of the present invention, the term “crystallizable fatty substance” means a deformable or not undeformable lipophilic compound which is solid at room temperature (25° C.) and atmospheric pressure, and which has a melting point greater than or equal to 25° C., preferably between 25° C. and 200° C., more preferentially between 25° C. and 120° C.

Some crystallizable fatty substances are commonly called waxes.

### The Wax(es)

According to a particular embodiment, composition C1 or C1′ of the invention comprises one or more waxes.

The term “wax” means a lipophilic compound, which is solid at room temperature (25° C.) and at atmospheric pressure, with a reversible solid/liquid change of state, having a melting point of greater than or equal to 30° C., which may be up to 200° C. and notably up to 120° C. In particular, the wax(es) that are suitable for use in the invention may have a melting point of greater than or equal to 45° C. and in particular greater than or equal to 55° C.

Within the meaning of the invention, the melting point corresponds to the temperature of the most endothermic peak observed on thermal analysis (DSC) as described in the standard ISO 11357-3; 1999. The melting point of the crystallizable fatty substance can be measured using a differential scanning calorimeter (DSC), for example the calorimeter sold under the name MDSC 2920 by TA Instruments. Such a measurement method is, for example, described in the document PCT/EP2013/062964.

According to a particular form of the invention, composition C1 or C1′ is solid, in particular anhydrous. It may then be in stick form; use will be made of polyethylene microwaxes in the form of crystallites with an aspect ratio at least equal to 2, and with a melting point ranging from 70° C. to 110° C. and preferably from 70° C. to 100° C., so as to reduce or even eliminate the presence of strata in the solid composition. These crystallites in needle form and notably the dimensions thereof may be characterized visually according to the above method.

### The Pasty Compound(s)

According to a particular embodiment, the composition of the invention comprises b) one or more pasty compounds.

For the purposes of the present invention, the term “pasty compound” means a lipophilic fatty compound that undergoes a reversible solid/liquid change of state, having anisotropic crystal organization in the solid state, and including, at a temperature of 23° C., a liquid fraction and a solid fraction.

Among the crystallizable fatty substances i) of fossil origin, mention may be made of: 1) paraffin wax, 2) ozokerite, 3) ceresin and 4) microcrystalline wax, notably comprising from 20 to 60 carbon atoms.

According to a particular embodiment of the invention, the crystallizable fatty substance(s) are of natural origin, particularly ii) of plant origin. Mention may be made of: 5) carnauba wax, 6) candelilla wax such as the product sold under the reference SP 75 G by Strahl & Pitsch, 7) laurel wax, notably oleander wax, 8) sugar cane wax, 9) ceramide, 10) esparto wax, 11) olive tree wax, 12) rice wax such as the product sold under the reference NC 1720 by Cera Rica Noda, 13) sunflower seed wax such as the product sold by Koster Keunen under the reference Sunflower wax, 14) hydrogenated jojoba wax, 15) hydrogenated castor oil, 16) hydrogenated olive oil, 17) hydrogenated cottonseed oil, 18) polyglyceryl-3 esters of green mimosa, jojoba and sunflower waxes, 19) absolute flower waxes such as blackcurrant flower essential wax, 20) soybean wax, 21) myrica fruit wax.

Other plant waxes may be used as crystallizable fatty substances b) of the invention, notably the following waxes: 22) caranday wax, 23) raffia wax, 24) Colombia wax, 25) esparto wax, 26) alfalfa wax, 27) bamboo wax, 28) hemp wax, 29) douglas fir wax, 30) cork wax, 31) sisal wax, 32) linseed wax, 33) cotton wax, 34) dammar wax, 35) cereal wax, 36) tea wax, 37) coffee wax, 38) ocatilla wax, 39) palm wax, 40) myrica wax, 41) bayberry wax, 42) ucuhuba wax, 43) Borneo wax, 44) Malabar wax, 45) illipe wax and 46) Japan tallow wax or Japan wax, preferably Japan wax.

According to a particular embodiment of the invention, the crystallizable fatty substance(s) b) are 46) Japan wax. Japan wax is a wax obtained from sumac (Rhus succedanea) berry seeds originating from Japan. It contains various fatty acids such as stearic acid, palmitic acid and heneicosylic acid, and a particular C21 acid.

According to another particular embodiment of the invention, the crystallizable fatty substance(s) are iii) of animal origin, notably chosen from 47) beeswax, 48) modified beeswax (cerabellina), 49) lanolin and 50) spermaceti.

In particular, the crystallizable fatty substance(s) are chosen from beeswax. In particular, beeswax comprises from 60% to 90%, notably from 70% to 80%, of wax esters, from 5% to 20% by weight, notably from 10% to 15% by weight, of free acids, between 5% and 30% by weight, notably from 10% to 20% of paraffins, alkenes, isoparaffins, cholesterol esters and pollen pigments are present in small amounts, and residues of volatile diesters and triesters (Ullmann's Encyclopedia of Industrial Chemistry, “Waxes”, E. Krendlinger, 2015 Wiley-VCH Verlag GmbH & Co KGaA, Weinheim10.1002/14356007.a28_103.pub2).

More specific beeswaxes may also be mentioned, such as ghedda wax (Asia Apis indica, A. florea, A. dorsata), which can be mixed with beeswaxes from India notably comprising between 50% and 95% by weight of wax esters, (notably 78% to 80% of wax esters), between 1% and 10% by weight (such as 4%) of glycerides (fats), between 2% and 8% by weight (notably between 5% and 6%) of free acids, between 6% and 10% by weight (notably between 8% and 9%) of hydrocarbons and between 0.5% and 4% by weight (notably from 1% to 2%) of partly resinous impurities. The acids notably include 7- and 16-hydroxypalmitic acids and 7,16-dihydroxymargaric acid.

Mention may also be made of 51) shellac wax from the resinous exudate of the insect Laccifer lacca (formerly Tachardia lacca) of the family Coccidae, which product is known as shellac, similar to beeswax, and is composed essentially of a mixture of C₁₂-C₁₈ even fatty acids (between 15% and 30% by weight, notably between 21% and 26% by weight of the total acid) and waxy acids (C₂₈-C₃₄, mainly C₃₂ and C₃₄). The specific component is trihydroxypalmitic acid (aleuritic acid), even though it is only present in small amounts, notably between 0.5% and 1.0%. Among the C₂₈-C₃₂ alcohols, the C₂₈ component predominates (between 50% and 70% by weight, notably between 62% and 65% of the total amount of alcohols). The main hydrocarbon-based components are paraffins containing 27, 29 and 31 carbon atoms (Ullmann's Encyclopedia of Industrial Chemistry, “Waxes”, E. Krendlinger, 2015 Wiley-VCH Verlag GmbH & Co. KGaA, Weinheim10.1002/14356007.a28_103.pub2).

The crystallizable fatty substance(s) may also be chosen from long-chain crystallizable alcohols and mixtures thereof, for instance cetearyl alcohol (50/50 C₁₆/C₁₈), stearyl alcohol, myristyl alcohol, cetyl alcohol and C₂₆-C₂₂ alcohols.

The crystallizable fatty substance(s) may also be chosen from long-chain crystallizable esters and mixtures thereof, such as the INCI compound “Cetyl esters (and) cetyl ester mixture of myristyl stearate and myristyl palmitate”, or the INCI compound “Myristyl stearate and myristyl palmitate mixture”, myristyl myristate, behenyl behenate, glycol distearate, glycol stearate, cetyl palmitate such as the commercial product Ercawax CP V/O from the supplier Erca, isopropyl palmitate, C₂₀-C₄₀ alkyl stearates, long-chain crystallizable esters of glycerol and mixtures thereof, for instance the compound sold under the name Compritol 888 CG ATO from Gattefosse (INCI: Glyceryl Dibehenate (and) Tribehenin (and) Glyceryl Behenate) or each of its components taken separately, tricaprin, trilaurin, trimyristin, tripalmitin, tristearin, glycerol distearate, glyceryl distearate, glyceryl dipalmitate/stearate and linoleoyl polyoxyl-6 glyceride.

In one embodiment of the invention, the crystallizable fatty substance(s) b) are chosen from fatty alcohols, glycol ester, glyceryl ester and glycerol ester. In one or more embodiments, the fatty substances comprise hydrogenated plant oil and glycol distearate.

The crystallizable fatty substance(s) may also be chosen from crystallizable fatty acids bearing a long alkyl chain and mixtures thereof, for instance the INCI compound “Stearic acid”, mixtures of stearic acid and palmitic acid, in particular from saturated C₄-C₂₈ fatty acids and unsaturated C₄-C₂₈ fatty acids.

The other crystallizable fatty substances that may be used according to the invention are notably marine waxes, synthetic beeswaxes, synthetic microcrystalline waxes, polyethylene waxes or polyolefin waxes in general, such as α-oligomers of olefins, for example the polymers Performa V® 825, 103 and 260 sold by New Phase Technologies, ethylene/propylene copolymers, such as Performalene® EP 700, or Fischer-Tropsch waxes or a mixture of these products.

Preferably, the crystallizable fatty substance is chosen from crystallizable mono/di/triesters derived from C₁₀-C₃₆ fatty alcohols and/or from C₁₂-C₃₈ fatty acids, and mixtures thereof.

According to a particular embodiment of the invention, the crystallizable fatty substance(s) are chosen from esters of C₁₀-C₃₀ fatty alcohols and/or of C₁₂-C₂₄ fatty acids, notably of formula (Ib), and also the hydrates

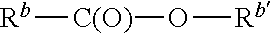

in which formula (Ib):
R^{b} and R^{b′}, which may be identical or different, preferably different, represent a linear or branched, preferably linear, saturated or unsaturated, preferably saturated, hydrocarbon-based chain comprising from 10 to 38 carbon atoms; preferably, R^{b′} represents a C₁₂-C₃₆ hydrocarbon-based chain, optionally substituted with one or more hydroxyl groups and/or optionally interrupted with one or more oxygen atoms, carbonyl or a combination thereof such as ester; preferably, R^{b} represents a C₁₂-C₃₈ hydrocarbon-based chain, which is in particular linear, saturated and unsubstituted, optionally interrupted with an ester group, and R^{b′} represents a C₁₂-C₃₀ chain, which is in particular linear, saturated and unsubstituted.

According to a particular embodiment of the invention, the crystallizable fatty substance(s) are chosen from the compounds of formula (Ib), notably chosen from cetyl palmitate or myricyl palmitate, and C₁₈-C₃₈ alkyl hydroxystearoyl stearate.

Preferably, the crystallizable fatty substance(s) b) are chosen from the compounds of formula (Ib′) below, and also the optical and geometric isomers thereof and the solvates thereof such as hydrates:

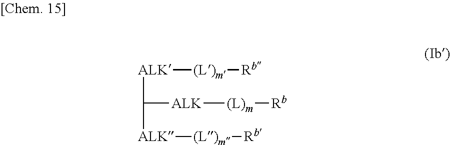

in which formula (Ib′):
   R^{b′}, R^{b″} and R^{b′″}, which may be identical or different, represent a hydrogen atom or a linear or branched, preferably linear, saturated or unsaturated, preferably saturated, hydrocarbon-based chain comprising from 10 to 30 carbon atoms, preferably a C₁₂-C₂₄ hydrocarbon-based chain, optionally substituted with one or more hydroxyl groups;
   ALK, ALK′, ALK″, which may be present or absent, and which may be identical or different, represent a linear or branched C₁-C₄ alkylene group, optionally substituted with at least one hydroxyl group; preferably, ALK′ and ALK″ represent a methylene group —CH₂—, and ALK is absent;
   L, L′ and L″, which may be identical or different, preferably identical, represent an ester group —C(O)—O— or —O—C(O)—, preferentially —O—C(O)—;
   m, m′ and m″ represent 0 or 1, preferably 1; it being understood that the three radicals R^{b′}, R^{b″} and R^{b′″} cannot simultaneously represent a hydrogen atom;
preferably, the crystallizable fatty substance(s) are chosen from compounds of formula (I′b) such as glyceryl behenate, glyceryl dibehenate, trihydroxystearin, tribehenin, and mixtures thereof.

According to an embodiment of the invention, the crystallizable fatty substance is present in a content between 0.01% and 30% by weight relative to the total weight of the composition, more particularly between 0.1% and 20% by weight, more particularly between 0.5% and 10% by weight, such as 5% by weight relative to the total weight of the composition.

Preferably, the crystallizable fatty substance is present in a content of between 0.1% and 15% by weight relative to the total weight of the composition, preferably between 0.5% and 12% by weight, preferentially between 1% and 8% by weight, advantageously between 2% and 7% by weight.

### c) The Oil(s)

According to a preferred embodiment of the invention, the composition C1 or C1′ also comprises one or more oils, i.e. one or more fatty substances that are liquid at room temperature (25° C.) other than the crystallizable fatty substance(s) b) as defined previously.

The oil(s) of the invention are of natural or synthetic origin, preferably natural, more preferentially of plant origin. They are different from fatty acids since salified fatty acids also constitute soaps which are generally soluble in aqueous media.

According to a preferred embodiment, the oil(s) c) are not mineral oils. Preferentially, composition C1 or C1′ does not comprise any mineral oil, such as liquid petroleum jelly.

Preferably, the composition contains one or more fatty substances c) which are hydrocarbon-based fatty substances that are liquid at 25° C. and atmospheric pressure.

According to a particular embodiment of the invention, composition C1 or C1′ contains one or more hydrocarbon-based oils chosen from C₆-C₁₆ hydrocarbons or hydrocarbons containing more than 16 carbon atoms and up to 60 carbon atoms, preferably between C₆ and C₁₆, and in particular alkanes, oils of animal origin, oils of plant origin, glycerides or fluoro oils of synthetic origin, fatty alcohols, fatty acid and/or fatty alcohol esters, and silicones. In particular, the oil(s) are chosen from non-silicone oils.

It is recalled that, for the purposes of the invention, the fatty alcohols, fatty esters and fatty acids more particularly contain one or more linear or branched, saturated or unsaturated hydrocarbon-based groups comprising 6 to 60 carbon atoms, which are optionally substituted, in particular with one or more hydroxyl groups OH (in particular from 1 to 4 hydroxyl groups). If they are unsaturated, these compounds may comprise one to three unsaturations, preferably from one to three conjugated or unconjugated carbon-carbon double bonds.

According to a particular embodiment of the invention, the oil(s) of the invention are chosen from C₆-C₁₆ alkanes, these compounds are linear or branched, and optionally cyclic; preferably, the oil(s) c) of the invention are chosen from linear or branched C₈-C₁₄, more preferentially C₉-C₁₃ and even more preferentially C₉-C₁₂ alkanes. Examples that may be mentioned include hexane, decane, undecane, dodecane, tridecane, and isoparaffins, for instance isohexadecane, isodecane or isododecane. The linear or branched hydrocarbons containing more than 16 carbon atoms may be chosen from liquid paraffins, liquid petroleum jelly, polydecenes, and hydrogenated polyisobutene such as Parleam®.

Among the hydrocarbon-based oil(s) c) having an overall solubility parameter according to the Hansen solubility space of less than or equal to 20 (MPa)_{1/2}, mention may be made of oils, which may be chosen from natural or synthetic, hydrocarbon-based oils, which are optionally fluorinated and optionally branched, alone or as a mixture.

According to a very advantageous embodiment, composition C1 or C1′ of the invention comprises one or more hydrocarbon-based oils. The hydrocarbon-based oil(s) may be volatile or non-volatile.

According to a preferred embodiment of the invention, the oil(s) c) are linear or branched hydrocarbon-based oils, which are volatile, notably chosen from undecane, decane, dodecane, isododecane, tridecane, and a mixture of various volatile oils thereof preferably comprising isododecane in the mixture, or a mixture of undecane and tridecane.

According to another particular embodiment, the oil(s) c) are a mixture of a volatile hydrocarbon-based oil and a non-volatile hydrocarbon-based oil, the mixture of which preferentially comprises dodecane or isododecane as volatile oil.

In particular, the oil(s) c) of the invention are a mixture of C₉-C₁₂ alkanes, preferably of natural origin, the chains of which comprise from 9 to 12 carbon atoms, preferably linear or branched C₉-C₁₂ alkanes. This mixture is notably known under the INCI name C₉-C₁₂ Alkane, CAS 68608-12-8, Vegelight Silk® sold by BioSynthls. This volatile biodegradable mixture of volatile oils is obtained from coconut oil (the viscosity is 0.9-1.1 cSt (40° C.) and it has a flash point of 65° C.).

According to one embodiment, composition C1 or C1′ contains at least 80% of hydrocarbon-based oils that are liquid at 25° C. and atmospheric pressure, which are preferably volatile, more preferentially chosen from isodecane, decane, Cetiol UT® and Vegelight Silk®.

According to another embodiment, composition C1 or C1′ comprises c) volatile and non-volatile oils, notably in a volatile oil/non-volatile oil ratio of greater than or equal to 4.

According to another embodiment, composition C1 or C1′ contains from 0 to 10% of silicone oils, preferably from 0 to 5% of silicone oils.

Volatile silicone oils that may be mentioned include volatile linear or cyclic silicone oils, notably those with a viscosity of less than or equal to 8 centistokes (cSt) (8×10₋₆ m²/s), and notably containing from 2 to 10 silicon atoms and in particular from 2 to 7 silicon atoms, these silicones optionally including alkyl or alkoxy groups containing from 1 to 10 carbon atoms. As volatile silicone oils that may be used in the invention, mention may notably be made of dimethicones with viscosities of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

As nonvolatile silicone oils, mention may be made of linear or cyclic nonvolatile polydimethylsiloxanes (PDMSs); polydimethylsiloxanes including alkyl, alkoxy and/or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenylmethyldiphenyltrisiloxanes, 2-phenylethyl trimethylsiloxysilicates and pentaphenyl silicone oils.

The hydrocarbon-based oil(s) c) of the invention are particularly chosen from:
hydrocarbon-based oils containing from 8 to 14 carbon atoms, and notably:
branched C₈-C₁₄ alkanes, for instance C₈-C₁₄ isoalkanes of petroleum origin (also known as isoparaffins), for instance isododecane (also known as 2,2,4,4,6-pentamethylheptane), isodecane and, for example, the oils sold under the trade names Isopar or Permethyl,
linear alkanes, for instance n-dodecane (C12) and n-tetradecane (C14) sold by Sasol under the respective references Parafol 12-97 and Parafol 14-97, and also mixtures thereof, the undecane-tridecane mixture, the mixtures of n-undecane (C11) and of n-tridecane (C13) obtained in examples 1 and 2 of patent application WO 2008/155059 from the company Cognis, and mixtures thereof, and also mixtures of n-undecane (C11) and of n-tridecane (C13) Cetiol Ultimate® from the company BASF,
short-chain esters (containing from 3 to 8 carbon atoms in total) such as ethyl acetate, methyl acetate, propyl acetate or n-butyl acetate,
hydrocarbon-based oils of plant origin such as triglycerides consisting of fatty acid esters of glycerol, the fatty acids of which may have various chain lengths ranging from C₄ to C₂₄, these chains possibly being linear or branched, and saturated or unsaturated; these oils are notably heptanoic acid or octanoic acid triglycerides, or alternatively wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cotton oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passion flower oil, musk rose oil or coconut oil; shea butter; or else caprylic/capric acid triglycerides, for instance those sold by the company Stearinerie Dubois or those sold under the names Miglyol 810®, 812® and 818® by the company Dynamit Nobel,
synthetic ethers containing from 10 to 40 carbon atoms,
linear or branched hydrocarbons of mineral or synthetic origin, such as petroleum jelly, polydecenes, hydrogenated polyisobutene such as Parleam®, squalane and liquid paraffins, and mixtures thereof,
esters such as the oils of formula R¹C(O)—O—R² in which R¹ represents a linear or branched fatty acid residue including from 1 to 40 carbon atoms and R² represents a, notably branched, hydrocarbon-based chain containing from 1 to 40 carbon atoms, on condition that R¹+R² is greater than or equal to 10, for instance purcellin oil (cetostearyl octanoate), isopropyl myristate, isopropyl palmitate, C₁₂ to C₁₅ alkyl benzoates, hexyl laurate, isodecyl neopentanoate, isostearyl neopentanoate, diisopropyl adipate, isononyl isononanoate, 2-ethylhexyl palmitate, isostearyl isostearate, 2-hexyldecyl laurate, 2-octyldecyl palmitate, 2-octyldodecyl myristate, alcohol or polyalcohol heptanoates, octanoates, decanoates or ricinoleates such as propylene glycol dioctanoate; hydroxylated esters such as isostearyl lactate, diisostearyl malate and 2-octyldodecyl lactate; polyol esters and pentaerythritol esters, more preferentially esters of a linear or branched C₈-C₁₀ fatty acid and of a linear or branched C₁₂-C₁₈ fatty alcohol alone or as a mixture with alkanes derived from the complete hydrogenation/reduction of fatty acids obtained from Cocos nucifera (coconut) oil, particularly dodecane or mixtures of cocoyl caprylate/caprate with dodecane; mention may be made of those having the INCI name Coconut alkanes (and) cocoyl caprylate/caprate sold under the name Vegelight 1212LC® by Grant Industries,
fatty alcohols that are liquid at room temperature, with a branched and/or unsaturated carbon-based chain containing from 12 to 26 carbon atoms, for instance octyldodecanol, isostearyl alcohol, oleyl alcohol, 2-hexyldecanol, 2-butyloctanol and 2-undecylpentadecanol;
carbonate oils which may be chosen from the carbonates of formula R8-O—CO—O—R9, with R8 and R9, which may be identical or different, representing a linear or branched C₄ to C₁₂ and preferentially C₆ to C₁₀ alkyl chain; the carbonate oils may be dicaprylyl carbonate (or dioctyl carbonate), sold under the name Cetiol CC® by the company BASF, bis(2-ethylhexyl) carbonate, sold under the name Tegosoft DEC® by the company Evonik, dipropylheptyl carbonate (Cetiol 4 All from BASF), dibutyl carbonate, dineopentyl carbonate, dipentyl carbonate, dineoheptyl carbonate, diheptyl carbonate, diisononyl carbonate or dinonyl carbonate, and preferably dioctyl carbonate;
volatile or non-volatile “ether oils” of formula R1-O—R2 in which R1 and R2 independently denote a linear, branched or cyclic C4-C24 alkyl group, preferably a C6-C18 alkyl group, and preferably a C8-C12 alkyl group. It may be preferable for R1 and R2 to be identical. Linear alkyl groups that may be mentioned include a butyl group, a pentyl group, a hexyl group, a heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group, an eicosyl group, a behenyl group, a docosyl group, a tricosyl group and a tetracosyl group. Branched alkyl groups that may be mentioned include a 1-methylpropyl group, a 2-methylpropyl group, a t-butyl group, a 1,1-dimethylpropyl group, a 3-methylhexyl group, a 5-methylhexyl group, an ethylhexyl group, a 2-ethylhexyl group, a 5-methyloctyl group, a 1-ethylhexyl group, a 1-butylpentyl group, a 2-butyloctyl group, an isotridecyl group, a 2-pentylnonyl group, a 2-hexyldecyl group, an isostearyl group, a 2-heptylundecyl group, a 2-octyldodecyl group, a 1,3-dimethylbutyl group, a 1-(1-methylethyl)-2-methylpropyl group, a 1,1,3,3-tetramethylbutyl group, a 3,5,5-trimethylhexyl group, a 1-(2-methylpropyl)-3-methylbutyl group, a 3,7-dimethyloctyl group and a 2-(1,3,3-trimethylbutyl)-5,7,7-trimethyloctyl group. Cyclic alkyl groups that may be mentioned include a cyclohexyl group, a 3-methylcyclohexyl group and a 3,3,5-trimethylcyclohexyl group. Advantageously, the ether oil is chosen from dicaprylyl ether, dicapryl ether, dilauryl ether, diisostearyl ether, dioctyl ether, nonyl phenyl ether, dodecyl dimethylbutyl ether, cetyl dimethylbutyl ether, cetyl isobutyl ether, and mixtures thereof. It is preferably chosen from dicaprylyl ether and dicapryl ether.

In addition to the hydrocarbon-based oil(s), composition C1 or C1′ of the invention may comprise a silicone oil. If silicone oil is in the composition of the invention, it is preferably in an amount which does not exceed 10% by weight relative to the weight of the composition, more particularly in an amount of less than 5% and more preferentially less than 2% by weight relative to the total weight of the composition.

In particular, composition C1 or C1′ comprises at least one hydrocarbon-based liquid fatty substance c) chosen from:
plant oils formed by fatty acid esters of polyols, in particular triglycerides, such as sunflower oil, sesame oil, rapeseed oil, macadamia oil, soybean oil, sweet almond oil, beauty-leaf oil, palm oil, grapeseed oil, corn oil, arara oil, cottonseed oil, apricot oil, avocado oil, jojoba oil, olive oil, coconut oil or cereal germ oil;
linear, branched or cyclic esters containing more than 6 carbon atoms, notably 6 to 30 carbon atoms; and notably isononyl isononanoate;
and more particularly esters of formula R^{d}—C(O)—O—R^{e} in which R^{d} represents a higher fatty acid residue including from 7 to 19 carbon atoms and R^{e} represents a hydrocarbon-based chain including from 3 to 20 carbon atoms, such as palmitates, adipates, myristates and benzoates, notably diisopropyl adipate and isopropyl myristate; more preferentially esters of formula R^{d}—C(O)—O—R^{e} in which R^{d} represents a higher fatty acid residue including from 8 to 10 carbon atoms and R^{e} represents a hydrocarbon-based chain including from 12 to 18 carbon atoms;
hydrocarbons and notably volatile or non-volatile linear, branched and/or cyclic alkanes, such as optionally volatile C₅-C₆₀ isoparaffins, such as undecane, dodecane, isododecane, tridecane, Parleam (hydrogenated polyisobutene), isohexadecane, cyclohexane, or Isopars, and mixtures thereof; or alkanes resulting from the complete hydrogenation/reduction of mixtures of fatty acids derived from Cocos nucifera (coconut) oil, such as dodecane, the mixture of C₉-C₁₂ alkanes, the chains of which comprise from 9 to 12 carbon atoms, preferably linear or branched C₉-C₁₂ alkanes, in particular comprising dodecane, or else liquid paraffin, liquid petroleum jelly, or hydrogenated polyisobutylene;
ethers containing 6 to 30 carbon atoms;
ketones containing 6 to 30 carbon atoms;
aliphatic fatty monoalcohols containing 6 to 30 carbon atoms, the hydrocarbon-based chain not including any substitution groups, such as oleyl alcohol, decanol, dodecanol, octadecanol, octyldodecanol and linoleyl alcohol;
polyols containing 6 to 30 carbon atoms, such as hexylene glycol; and
mixtures thereof, such as mixtures of esters of linear or branched C₈-C₁₀ fatty acid and C₁₂-C₁₈ fatty alcohol and alkanes resulting from the complete hydrogenation/reduction of mixtures of fatty acids from Cocos nucifera (coconut) oil, in particular dodecane, such as mixtures of cocoyl caprylate/caprate and dodecane; mention may be made of those having the INCI name Coconut alkanes (and) coco-caprylate/caprate sold under the name Vegelight 1212LC® by Grant Industries; or mixtures of C₉-C₁₂ alkanes, the chains of which comprise from 9 to 12 carbon atoms, preferably linear or branched C₉-C₁₂ alkanes, in particular comprising dodecane; mention may be made of the oil mixture having the INCI name C9-12 Alkane, Vegelight Silk® sold by BioSynthls.

Preferably, composition C1 or C1′ of the invention comprises at least one hydrocarbon-based oil c) chosen from:
plant oils formed by fatty acid esters of polyols, in particular triglycerides,
esters of formula R^{d}—C(O)—O—R^{e} in which R^{d} represents a higher fatty acid residue including from 7 to 19 carbon atoms and R^{e} represents a hydrocarbon-based chain including from 3 to 20 carbon atoms, more preferentially esters of formula R^{d}—C(O)—O—R^{e} in which R^{d} represents a higher fatty acid residue including from 8 to 10 carbon atoms and R^{e} represents a hydrocarbon-based chain including from 12 to 18 carbon atoms;
volatile or non-volatile, linear or branched C₉-C₁₂ alkanes, such as isododecane and alkanes resulting from the complete hydrogenation/reduction of mixtures of fatty acids obtained from Cocos nucifera (coconut) oil, in particular dodecane;
volatile or non-volatile, non-aromatic cyclic C₅-C₁₂ alkanes;
ethers containing 7 to 30 carbon atoms;
ketones containing 8 to 30 carbon atoms;
aliphatic fatty monoalcohols containing 12 to 30 carbon atoms, the hydrocarbon-based chain not including any substitution groups; and
mixtures thereof.

Advantageously, the oil(s) c) of the invention are apolar, i.e. formed solely of carbon and hydrogen atoms.

The hydrocarbon-based oil(s) are preferably chosen from hydrocarbon-based oils containing from 8 to 14 carbon atoms, which are in particular volatile, more particularly the apolar oils described previously.

Preferentially, the oil(s) c) of the invention are chosen from alkanes such as dodecane, decane, isododecane, fatty alcohols such as octyldodecanol, esters such as isononyl isononanoate, cocoyl caprylate/caprate and mixtures thereof, more preferentially alkanes.

More particularly, the oil(s) c) of the invention are chosen from linear or branched C₆-C₁₆, preferably C₈-C₁₄, more preferentially C₉-C₁₃ and even more preferentially C₉-C₁₂ alkanes, and even more preferentially the alkanes are volatile. More particularly, the oil(s) c) of the invention are volatile and are chosen from undecane, decane, dodecane, isododecane, tridecane, and a mixture thereof notably comprising dodecane, isododecane or a mixture of undecane and tridecane.

Preferentially, the oil(s) c) of the invention are isododecane.

According to another advantageous embodiment of the invention, the oil(s) c) of the invention are a mixture of non-volatile oil(s) and volatile oil(s); preferably, the mixture comprises, as volatile oil, undecane, dodecane, isododecane or tridecane, more preferentially isododecane. A mixture of volatile oil and non-volatile oil that may be mentioned is the mixture of isododecane and of isononyl isononanoate or the mixture of isododecane with isononyl isononanoate.

More preferentially, when the oil(s) are a mixture of volatile oil and of non-volatile oil, the amount of volatile oil is greater than the amount of non-volatile oil.

In particular, in the mixture, the non-volatile oil c) is a phenyl silicone oil, preferably chosen from pentaphenyl silicone oils.

Advantageously, composition C₁ or C₁′ comprises one or more oils, in a content ranging from 2% to 99.9% by weight, relative to the total weight of said composition, preferably ranging from 5% to 90% by weight, preferably ranging from 10% to 80% by weight, preferably ranging from 20% to 80% by weight, relative to the total weight of said composition.

According to a preferred embodiment of the invention, composition C₁ or C₁′ according to the invention comprises c) one or more fatty substances that are notably liquid at 25° C. and at atmospheric pressure, optionally e) water and d) one or more organic solvents other than c).

d) Organic Solvent(s) Other than c)

According to a particular embodiment of the invention, composition C1 or C1′ also comprises one or more organic solvents other than c), which are apolar or polar, preferably polar, and which are protic or aprotic, more particularly protic and/or polar, preferably protic and polar.

Preferably, the organic solvent(s) are water-miscible.

According to the present invention, the term “water-miscible solvent” denotes a compound that is liquid at room temperature and water-miscible (miscibility with water of greater than 50% by weight at 25° C. and atmospheric pressure).

The organic solvent(s) that may be used in composition C1 or C1′ of the invention may also be volatile.

Among the organic solvents that may be used in composition C1 or C1′ according to the invention, mention may notably be made of polar protic or polar aprotic organic solvents, preferably polar protic organic solvents, particularly lower monoalcohols containing from 2 to 10 carbon atoms, such as ethanol and isopropanol, preferably ethanol.

According to one embodiment, composition C1 or C1′ of the invention comprises one or more organic solvents, preferably chosen from monoalcohols containing from 2 to 6 carbon atoms such as ethanol and isopropanol.

Preferably, the amount of organic solvent(s) is less than 70% by weight, more preferentially less than 50% by weight, relative to the total weight of composition C1 or C1′. According to one embodiment of the invention, composition C1 or C1′ comprises an amount of organic solvent(s) of greater than 0.5%, more particularly greater than or equal to 1% by weight relative to the total weight of the composition. In particular, the composition comprises between 2% and 50% by weight of organic solvent(s).

### e) Water

According to a particular embodiment of the invention, composition C1 or C1′ also comprises water.

The water that is suitable for use in the invention may be tap water, distilled water, spring water, a floral water such as cornflower water and/or a mineral water such as Vittel water, Lucas water or La Roche Posay water and/or a thermal water.

According to one embodiment, composition C1 or C1′ of the invention comprises e) water and at least one fatty substance c).

According to a particular embodiment of the invention, composition C1 or C1′ comprises an amount of water of less than or equal to 5% by weight relative to the total weight of the composition, particularly less than or equal to 2% by weight, preferably less than 1% by weight, more preferentially less than 0.5% by weight relative to the total weight of the composition. More particularly, the composition of the invention is anhydrous, i.e. free of water. Where appropriate, such small amounts of water may notably be introduced by ingredients of the composition that may contain residual amounts thereof.

### f) The Surfactants

According to a particular embodiment of the invention, composition C1 or C1′ also comprises f) one or more surfactants, preferably nonionic or ionic surfactants, or mixtures thereof.

According to another particular embodiment of the invention, composition C1 or C1′ does not comprise any surfactant.

The term “surfactant” means a compound which modifies the surface tension between two surfaces. The surfactant(s) d) are amphiphilic molecules, which have two parts of different polarity, one part being lipophilic (which retains fatty substances) which is apolar, the other hydrophilic part (miscible or soluble in water) being polar. The lipophilic part is generally a fatty chain, and the other water-miscible part is polar, and/or protic.

The term “ionic” means anionic, cationic, amphoteric or zwitterionic.

The term “fatty chain” means a linear or branched, saturated or unsaturated hydrocarbon-based chain comprising more than 6 atoms, preferably between 6 and 30 carbon atoms and preferably from 8 to 24 carbon atoms.

According to a first particular embodiment, the composition of the invention contains d) at least one silicone or non-silicone nonionic surfactant.

Among the nonionic surfactants according to the invention, mention may be made, alone or as mixtures, of fatty alcohols, α-diols and alkylphenols, these three types of compound being polyethoxylated, polypropoxylated and/or polyglycerolated and containing a fatty chain comprising, for example, 8 to 22 carbon atoms, the number of ethylene oxide or propylene oxide groups possibly ranging in particular from 2 to 50 and the number of glycerol groups possibly ranging in particular from 2 to 30. Mention may also be made of ethylene oxide and propylene oxide copolymers, condensates of ethylene oxide and of propylene oxide with fatty alcohols; polyethoxylated fatty amides preferably having from 2 to 30 ethylene oxide units, polyglycerolated fatty amides containing on average 1 to 5, and in particular 1.5 to 4, glycerol groups, ethoxylated fatty acid esters of sorbitan containing from 2 to 30 ethylene oxide units, fatty acid esters of sucrose, fatty acid esters of polyethylene glycol, alkylpolyglycosides, N-alkylglucamine derivatives, amine oxides such as (C₁₀-C₁₄)alkylamine oxides or N-acylaminopropylmorpholine oxides.

The surfactant(s) represent in total particularly from 0.01% to 30% by weight, preferably from 0.5% to 15% by weight, even more preferentially from 1% to 5% by weight and better still between 1% and 5% by weight of the composition, relative to the total weight of the composition.

### Form of the Composition:

According to one embodiment of the invention, the composition comprises an aqueous phase. The composition is notably formulated as water-in-oil or oil-in-water emulsions or as multiple emulsions (oil-in-water-in-oil or water-in-oil-in-water triple emulsions (such emulsions are known and described, for example, by C. Fox in “Cosmetics and Toiletries”—November 1986—Vol. 101—pages 101-112)).

According to a particular embodiment of the invention, the composition is a direct emulsion, i.e. an emulsion of oil-in-water or O/W type. The weight amount of oil is preferably less than 70% in the inverse emulsion, preferably less than or equal to 40%, more particularly less than or equal to 35% by weight relative to the total weight of the composition.

More particularly, in the direct emulsion, the amount of water is greater than or equal to 30% by weight relative to the total weight of the composition, more particularly greater than or equal to 40%, preferentially greater than or equal to 35%.

According to another particular embodiment of the invention, the composition of the invention is an inverse emulsion, i.e. of water-in-oil or W/O type. The weight amount of oil is preferably greater than 30% in the inverse emulsion, preferably greater than 40%, more preferentially greater than or equal to 45% by weight relative to the total weight of the composition. More particularly, in the inverse emulsion, the amount of water is less than 40% by weight relative to the total weight of the composition, preferably less than or equal to 30% by weight.

Composition C1 or C1′ according to the invention preferably has a pH ranging from 3 to 9, depending on the support chosen.

According to a particular embodiment of the invention, the pH of the composition(s) is neutral or even slightly acidic. Preferably, the pH of the composition is between 6 and 7. The pH of these compositions may be adjusted to the desired value by means of acidifying or basifying agents usually used in cosmetics, or alternatively using standard buffer systems.

The term “basifying agent” or “base” means any agent for increasing the pH of the composition in which it is present. The basifying agent is a Bronsted, Lowry or Lewis base. It may be mineral or organic. Particularly, said agent is chosen from a) aqueous ammonia, b) (bi)carbonate, c) alkanolamines such as monoethanolamine, diethanolamine, triethanolamine and derivatives thereof, d) oxyethylenated and/or oxypropylenated ethylenediamines, e) organic amines, f) mineral or organic hydroxides, g) alkali metal silicates such as sodium metasilicates, h) amino acids, preferably basic amino acids such as arginine, lysine, ornithine, citrulline and histidine, and i) the compounds of formula (F) below:

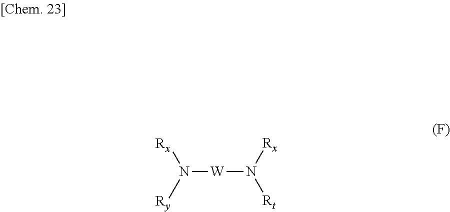

in which formula (F):
   W is a divalent C₁-C₆ alkylene radical optionally substituted with one or more hydroxyl groups or a C₁-C₆ alkyl radical, and/or optionally interrupted with one or more heteroatoms such as O or NRᵤ;
   Rₓ, R_{y}, R_{z}, Rₜ and Rᵤ, which may be identical or different, represent a hydrogen atom or a C₁-C₆ alkyl, C₁-C₆ hydroxyalkyl or C₁-C₆ aminoalkyl radical.

Examples of amines of formula (F) that may be mentioned include 1,3-diaminopropane, 1,3-diamino-2-propanol, spermine and spermidine.

The term “alkanolamine” means an organic amine comprising a primary, secondary or tertiary amine function, and one or more linear or branched C₁-C₈ alkyl groups bearing one or more hydroxyl radicals.

Among the mineral or organic hydroxides, mention may be made of those chosen from a) hydroxides of an alkali metal, b) hydroxides of an alkaline-earth metal, for instance sodium hydroxide or potassium hydroxide, c) hydroxides of a transition metal, d) hydroxides of lanthanides or actinides, quaternary ammonium hydroxides and guanidinium hydroxide. The mineral or organic hydroxides a) and b) are preferred.

Mention may be made, among the acidifying agents for the compositions used in the invention, by way of example, of mineral or organic acids, such as hydrochloric acid, orthophosphoric acid, sulfuric acid, carboxylic acids, such as acetic acid, tartaric acid, citric acid or lactic acid, or sulfonic acids.

The basifying agents and the acidifying agents as defined previously preferably represent from 0.001% to 20% by weight relative to the weight of the composition. and more particularly from 0.005% to 8% by weight of the composition.

According to a particular embodiment of the invention, composition C1 or C1′ comprises an amount of water of less than or equal to 10% by weight relative to the total weight of the composition. Even more preferentially, composition C1 or C1′ comprises an amount of water of less than or equal to 5%, better still less than 2%, even better still less than 0.5%, and is notably free of water. Where appropriate, such small amounts of water may notably be introduced by ingredients of the composition that may contain residual amounts thereof.

According to a particular embodiment of the invention, the composition does not comprise any water.

Advantageously, composition C1 or C1′ of the invention comprises a physiologically acceptable medium. In particular, the composition is a cosmetic composition.

The term “physiologically acceptable medium” means a medium that is compatible with human keratin materials, for instance the skin, the lips, the nails, the eyelashes, the eyebrows or the hair.

The term “cosmetic composition” means a composition that is compatible with keratin materials, which has a pleasant colour, odour and feel and which does not cause any unacceptable discomfort (stinging or tautness) liable to discourage the consumer from using it.

The term “keratin materials” means the skin (body, face, contour of the eyes, scalp), head hair, the eyelashes, the eyebrows, bodily hair, the nails or the lips.

Composition C1 or C1′ according to the invention may comprise one or more cosmetic additives chosen from fragrances, preserving agents, fillers, colouring agents, UV-screening agents, moisturizers, vitamins, ceramides, antioxidants, free-radical scavengers, polymers other than a), thickeners or film-forming agents other than b), trace elements, softeners, sequestrants, agents for combating hair loss, anti-dandruff agents, propellants. In particular, composition C1 or C1′ according to the invention also comprises one or more colouring agents chosen from pigments, direct dyes and mixtures thereof, preferably pigments; more preferentially, the pigment(s) of the invention are chosen from carbon black, iron oxides, notably black iron oxides, and micas coated with iron oxide, triarylmethane pigments, notably blue and violet triarylmethane pigments, such as Blue 1 Lake, azo pigments, notably red azo pigments, such as D&C Red 7, an alkali metal salt of lithol red, such as the calcium salt of lithol red B, even more preferentially red iron oxides.

Advantageously, composition C1 or C1′ according to the invention is a makeup composition, in particular a lip makeup composition, a mascara, an eyeliner, an eye shadow or a foundation.

### The Adjuvants

Composition C1 or C1′ according to the invention may also comprise one or more fillers, notably in a content ranging from 0.01% to 30% by weight and preferably ranging from 0.01% to 20% by weight relative to the total weight of the composition. The term “fillers” should be understood as meaning colourless or white, mineral or synthetic particles of any shape, which are insoluble in the medium of the composition, irrespective of the temperature at which the composition is manufactured. These fillers notably serve to modify the rheology or texture of the composition.

Composition C1 or C1′ according to the invention may be in the form of an anhydrous composition, a water-in-oil emulsion or an oil-in-water emulsion.

The invention is illustrated in greater detail in the examples that follow. The amounts are indicated as weight percentages.

### EXAMPLES

The PHAs illustrated in the various examples were prepared in 3-litre chemostats and/or 5-litre Fernbach flasks depending on whether or not a β-oxidation pathway inhibitor was used. The isolation of the PHAs is similar for all the examples obtained.

In a first step, the microorganism generates the PHAs which are stored in intracellular granules, the proportion of which varies as a function of the applied conditions such as the temperature or the nature of the culture medium. The generation of PHA granules may or may not be associated with the growth of the microorganism as a function of the nature of the microorganisms. During the second step, the biomass containing the PHAs is isolated, i.e. separated from the fermentation medium, and then dried. The PHAs are extracted from the biomass before being purified, if necessary.

A mixture of saturated and unsaturated carbon sources is, for certain examples, necessary for the stability of the PHA obtained.

TABLE 2

| Carbon source | CAS |
|---|---|
| Caprylic acid (RADIACID 608) | 124-07-2 |
| Nonanoic acid | 112-05-0 |
| Undecylenic acid (10-Undeoenoic acid) | 112-38-9 |

TABLE 3

| Carbon source | Genus and species | Source |
|---|---|---|
| Mixture of caprylic acid and | Pseudomonas | ATCC ® 47054 ™ |
| undecylenic acid | putida | |
| Mixture of nonanoic acid and | Pseudomonas | ATCC ® 47054 ™ |
| undecylenic acid | putida | |

### Example 1: PHA Bearing a Side Chain R¹ Representing a Linear 10% Unsaturated n-Octenyl Group and R² Representing an n-Pentyl Group

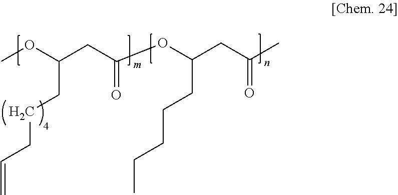

The process for synthesizing the compound of Example 1 is adapted from the article: Fed-batch production of unsaturated medium-chain-length polyhydroxyalkanoates with controlled composition by Pseudomonas putida KT2440, Z. Sun, J. A. Ramsay, M. Guay, B. A. Ramsay, Applied Microbiology Biotechnology, 82. 657-662, 2009.

The microorganism used is Pseudomonas putida KT2440 ATCC® 47054™. The culture method is performed under fed-batch growth axenic conditions with a maintenance solution containing a mixture of carbon source at a rate μ=0.15 h⁻¹ in a 3 L chemostat containing 2.5 L of culture medium.

The system is aerated with a flow of 0.5 vvm of air for a nominal dissolved oxygen (O_{D}) value at 30% of saturation. The pH is regulated with 15% aqueous ammonia solution. The temperature of the fermentation medium is regulated at 30° C.

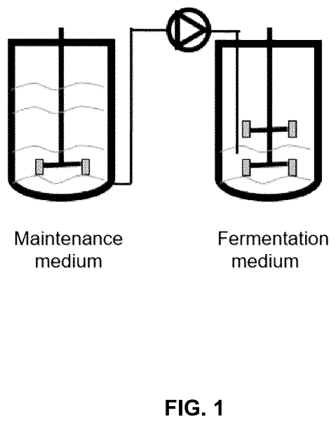

Equipment for the fed-batch growth fermentation mode:
The fermentation medium is regulated in terms of temperature-pressure of dissolved oxygen and pH (not shown): see the attached FIG. 1.
The production process is performed using three different culture media. The first culture medium, defined CM1 “inoculum”, is used for the preparation of the preculture. The second culture medium, defined CM2 “batch”, is used for unfed batch growth of the microorganism with the primary carbon sources in the Fernbach flasks. The third culture medium, defined CM3 “maintenance”, is used for the fed-batch or maintenance fermentation mode with the carbon sources of interest at a flow rate calibrated as a function of the growth of the microorganism.

TABLE 4

| Ingredients in grams | CM1 | CM2 | CM3 |
|---|---|---|---|
| per litre | <<inoculum>> | <<batch>> | <<maintenance>> |
| (NH₄)₂SO₄ | 4.7 | 4.7 | |
| Na₂HPO₄•7H₂O | 12 | 9 | |
| KH₂PO₄ | 2.7 | 2.03 | |
| MgSO₄•7H₂O | 0.8 | 1.03 | |
| Nutrient broth | 3 | / | |
| Caprylic acid | / | 0.9 | 900 |
| Undecylenic acid | / | 0.1 | 100 |
| Microelement solution | / | 10 | |
| Acrylic acid | / | / | |

| | |
|---|---|
| 2N NaOH | qs pH = 6.8 |
| MilliQ water | qs 1000 g |

The composition of the Nutrient Broth, as mass percentages, is 37.5% beef extract and 62.5% peptone. Reference 233000 DIFCO™.

TABLE 5

| Ingredients in grams | | |
|---|---|---|
| per litre | Amount | |
| FeSO₄•7H₂O | 10.0 | g |
| CaCl₂•2H₂O | 3.0 | g |
| ZnSO₄•7H₂O | 2.2 | g |
| MnSO₄•4H₂O | 0.5 | g |
| H₃BO₃ | 0.3 | g |
| CoCl₂•6H₂O | 0.2 | g |
| Na₂MoO₄•2H₂O | 0.15 | g |
| NiCl₂•6H₂O | 0.02 | g |
| CuSO₄•5H₂O | 1.00 | g |

| | |
|---|---|
| MilliQ water (or 0.5N HCL) | qs 1000 g |

100 mL of preculture are prepared by suspending a cryotube containing 1 mL of the strain with 100 mL of “inoculum” culture medium at a pH adjusted to 6.8 with 2N NaOH in a 250 mL Fernbach flask and are then incubated at 30° C. at 150 rpm for 24 hours. 1.9 L of CM2 “batch” culture medium placed in a presterilized 3 L chemostat are inoculated at O_{D}=0.1 with the 100 mL of preculture. After 4 hours at 30° C. at 850 rpm.

At the end of the introduction, the biomass is isolated by centrifugation and then washed three times with water. The biomass is dried by lyophilization before being extracted with ethyl acetate for 24 hours. The suspension is clarified by filtration on a GF/A filter (Whatman®). The filtrate, composed of PHA dissolved in the ethyl acetate, is concentrated by evaporation and then dried under high vacuum at 40° C. to constant mass.

The PHA may optionally be purified by successive dissolution and precipitation from an ethyl acetate/ethanol 70% methanol system, for example.

The PHA was fully characterized by spectroscopic and spectrometric methods and is in accordance with the expected chemical structure.

### Preparation of Example 1′: PHA Copolymer Bearing a Side Chain R¹ Representing a 5% Unsaturated n-Octenyl Group and R² Representing an n-Hexyl Group

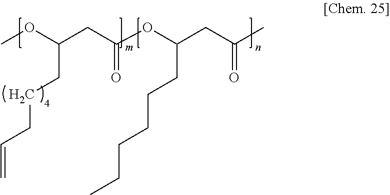

The copolymer of Example 1′ (5% unsaturation and R² chain representing n-hexyl) was prepared according to the procedure described for Example 1, with the same composition of the microelement solution as described in Example 1 and with the following culture medium compositions:

TABLE 6

| Ingredients in | CM1 | CM2 | CM3 |
|---|---|---|---|
| grams per litre | “inoculum” | “batch” | “maintenance” |
| (NH₄)₂SO₄ | 4.7 | 4.7 | |
| Na₂HPO₄•7H₂O | 12 | 9 | |
| KH₂PO₄ | 2.7 | 2.03 | |
| MgSO₄•7H₂O | 0.8 | 1.03 | |
| Nutrient broth | 3 | / | |
| Nonanoic acid | / | 0.95 | 950 |
| Undecylenic acid | | 0.05 | 50 |
| Microelement solution | / | 10 | |

| | |
|---|---|
| 2N NaOH | qs pH = 6.8 |
| MilliQ water | qs 1000 g |

The PHA copolymer of Example 1′ was fully characterized by spectroscopic and spectrometric methods and is in accordance with the expected chemical structure, with a degree of unsaturation of 5%.

### Example 1″: PHA Copolymer Bearing a Side Chain R¹ Representing a Linear 10% Unsaturated n-Octenyl Group and R² Representing an n-Hexyl Group

The copolymer of Example 1″ (10% unsaturation and R² chain representing n-hexyl) was prepared according to the procedure described for Example 1, with the same composition of the microelement solution as described in Example 1 and with the following culture medium compositions:

TABLE 7

| Ingredients in | CM1 | CM2 | CM3 |
|---|---|---|---|
| grams per litre | “inoculum” | “batch” | “maintenance” |
| (NH₄)₂SO₄ | 4.7 | 4.7 | / |
| Na₂HPO₄•7H₂O | 12 | 9 | / |
| KH₂PO₄ | 2.7 | 2.03 | / |
| MgSO₄•7H₂O | 0.8 | 1.03 | / |
| Nutrient broth | 3 | / | / |
| Nonanoic acid | / | 0.90 | 900 |
| Undecylenic acid | / | 0.1 | 100 |
| Microelement solution | / | 10 | / |

| | |
|---|---|
| 2N NaOH | qs pH = 6.8 |
| MilliQ water | qs 1000 g |

The PHA was fully characterized by spectroscopic and spectrometric methods and is in accordance with the expected chemical structure.

### Example 1′″: PHA Copolymer Bearing a Side Chain R¹ Representing a Linear 30% Unsaturated n-Octenyl Group and R² Representing an n-Pentyl Group

The copolymer of Example 1′″ (30% unsaturated and R² chain representing n-pentyl) was prepared according to the procedure described for Example 1, with the same composition of the microelement solution as described in Example 1 and with the following culture medium compositions:

TABLE 8

| Ingredients in | CM1 | CM2 | CM3 |
|---|---|---|---|
| grams per litre | “inoculum” | “batch” | “maintenance” |
| (NH₄)₂SO₄ | 4.7 | 4.7 | / |
| Na₂HPO₄•7H₂O | 12 | 9 | / |
| KH₂PO₄ | 2.7 | 2.03 | / |
| MgSO₄•7H₂O | 0.8 | 1.03 | / |
| Nutrient broth | 3 | / | / |
| Octanoic acid | / | 0.70 | 700 |
| Undecylenic acid | / | 0.3 | 300 |
| Microelement solution | / | 10 | / |

| | |
|---|---|
| 2N NaOH | qs pH = 6.8 |
| MilliQ water | qs 1000 g |

The PHA copolymer was fully characterized by spectroscopic and spectrometric methods and is in accordance with the expected chemical structure.

### Example 2: Poly(3-hydroxyoctanoate-co-undecenoate) Containing 10% Unsaturations 100% Grafted with Thiolactic Acid (Compound of Example 1 Grafted with Thiolactic Acid TLA)

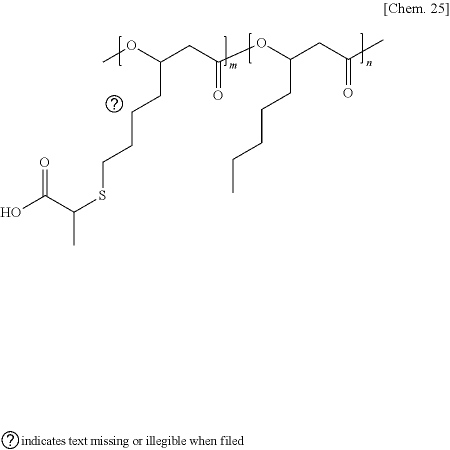

1 g of the compound of Example 1 and 150 mg of thiolactic acid were dissolved in 20 mL of ethyl acetate at room temperature with stirring. 20 mg of 2,2-dimethoxy-2-phenylacetophenone (Irgacure 651) were added to the mixture. The medium was then irradiated under a 100 W UV lamp at 365 nm (reference) and with stirring for at least 10 minutes.

20 mL of the reaction medium were then precipitated from a 200 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 2 was fully characterized by spectroscopic and spectrometric methods and is in accordance with the expected chemical structure.

### Example 3: Poly(3-hydroxyoctanoate-co-undecenoate) Containing 10% Unsaturations 100% Grafted with Octanethiol (Compound of Example 1 Grafted with n-Octanethiol)

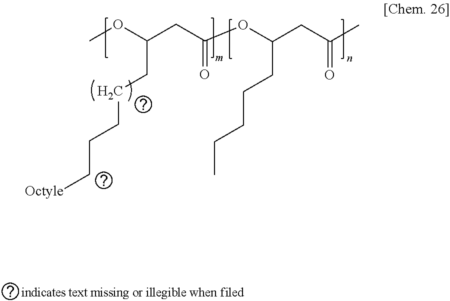

0.5 g of the compound of Example 1 and 125 mg of octanethiol were dissolved in 10 mL of ethyl acetate at room temperature with stirring. 15 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 100 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 3 was fully characterized by spectroscopic and spectrometric methods and is in accordance with the expected chemical structure.

### Example 4: Poly(3-hydroxyoctanoate-co-undecenoate) Containing 10% Unsaturations 75% Grafted with 8-Mercapto-1-Octanol (Compound of Example 1 Grafted with 8-Mercapto-1-Octanol)

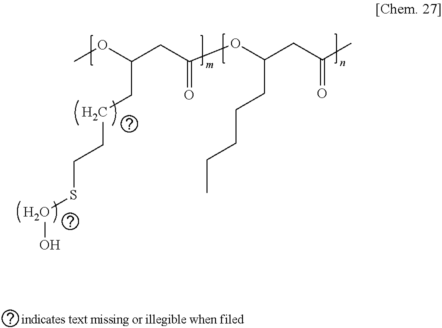

50 mg of the compound of Example 1 and 10 mg of 8-mercapto-1-octanol were dissolved in 5 mL of ethyl acetate at room temperature with stirring. 2 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 50 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 4 was fully characterized by spectroscopic and spectrometric methods and is in accordance with the expected chemical structure. Grafting to 75% or 7.5% of functions in total.

### Example 5: Poly(3-hydroxyoctanoate-co-undecenoate) Containing 10% Unsaturations 32% Grafted with Cysteamine (Compound of Example 1 Grafted with Cysteamine)

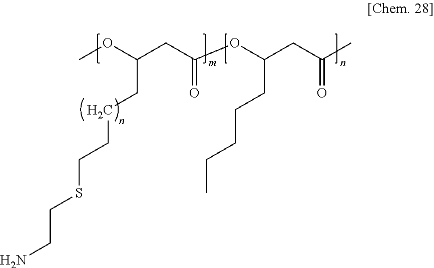

0.5 g of the compound of Example 1 and 54 mg of cysteamine were dissolved in a mixture of 10 mL of dichloromethane and 2 mL of ethanol at room temperature with stirring. 10 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 100 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 5 was fully characterized by spectroscopic and spectrometric methods and is in accordance with the expected chemical structure. Grafting to 32% or 3.2% of functions in total.

### Example 6: Poly(3-hydroxyoctanoate-co-undecenoate) Containing 10% Unsaturations 73% Grafted with Cyclohexanethiol (CHT) (Compound of Example 1 Grafted with CHT)

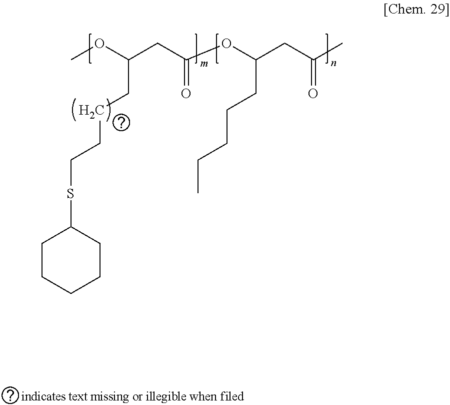

100 mg of the compound of Example 1 and 26 mg of cyclohexanethiol were dissolved in 5 mL of dichloromethane at room temperature with stirring. 5 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 50 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 6 was fully characterized by spectroscopic and spectrometric methods and is in accordance with the expected chemical structure. Grafting to 73% or 7.3% of functions in total.

### Example 7: Poly(3-hydroxyoctanoate-co-undecenoate) Containing 10% Unsaturations 70% Grafted with 2-Furanmethanethiol (FT) (Compound of Example 1 Grafted with FT)

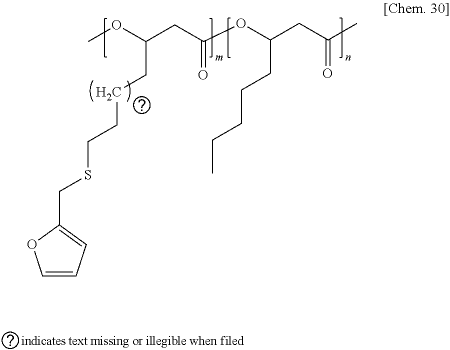

100 mg of the compound of Example 1 and 26 mg of 2-furanmethanethiol were dissolved in 5 mL of dichloromethane at room temperature with stirring. 5 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 50 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 7 was fully characterized by spectroscopic and spectrometric methods and is in accordance with the expected chemical structure. Grafting to 70% or 7% of functions in total.

### Example 8: Poly(3-hydroxyoctanoate-co-undecenoate) Containing 10% Unsaturations 70% Grafted with 1-Thio-(3-D-Glucose Tetraacetate (Compound of Example 1 Grafted with TG 7)

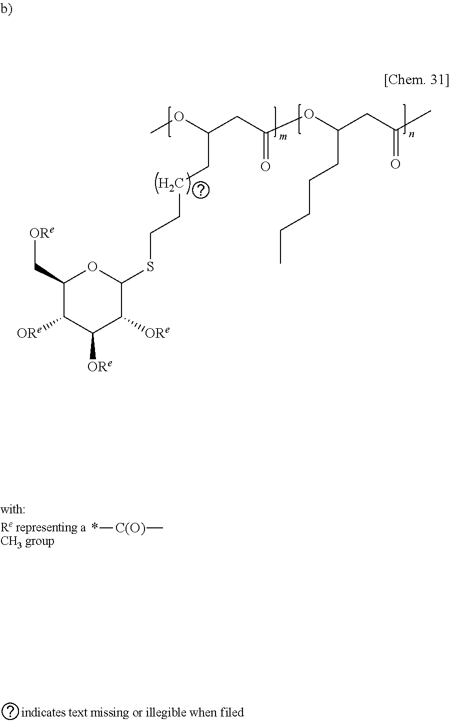

100 mg of the compound of Example 1 and 26 mg of 1-thio-β-D-glucose tetraacetate were dissolved in 5 mL of dichloromethane at room temperature with stirring. 5 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 50 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 8 was fully characterized by spectroscopic and spectrometric methods and is in accordance with the expected chemical structure. Grafting to 70% or 7% of functions in total.

### Example 9: Poly(3-hydroxyoctanoate-co-undecenoate) Containing 10% Unsaturations 50% Grafted with 2-Phenylethanethiol (PT) (Compound of Example 1 Grafted with PT)

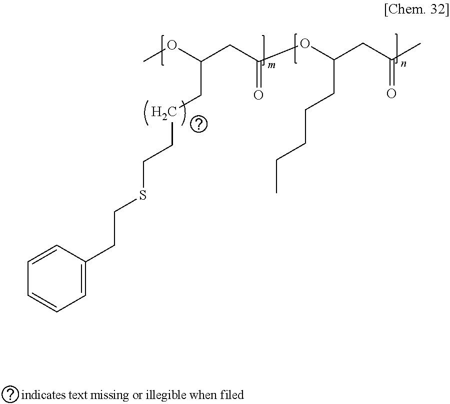

100 mg of the compound of Example 1 and 26 mg of 2-phenylethanethiol were dissolved in 5 mL of dichloromethane at room temperature with stirring. 5 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 50 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 9 was fully characterized by spectroscopic and spectrometric methods and is in accordance with the expected chemical structure. Grafting to 50% or 5% of functions in total.

### Example 10: Poly(3-hydroxyoctanoate-co-undecenoate) Containing 10% Unsaturations 64% Grafted with 4-Tert-Butylbenzyl Mercaptan (TBM) (Compound of Example 1 Grafted with TBM)

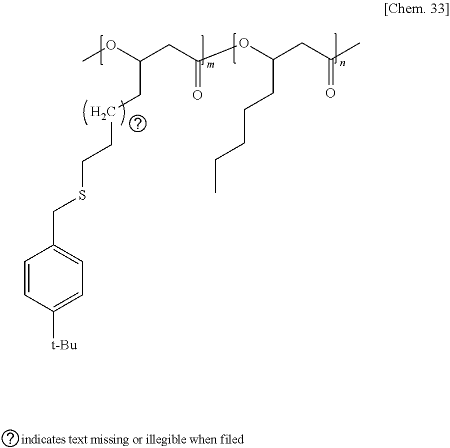

100 mg of the compound of Example 1 and 26 mg of 4-tert-butylbenzyl mercaptan were dissolved in 5 mL of dichloromethane at room temperature with stirring. 5 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 50 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 10 was fully characterized by spectroscopic and spectrometric methods and is in accordance with the expected chemical structure. Grafting to 64% or 6.4% of functions in total.

### Example 11: Poly(3-hydroxynonanoate-co-undecenoate) Containing 10% Unsaturations 100% Grafted with Thiolactic Acid

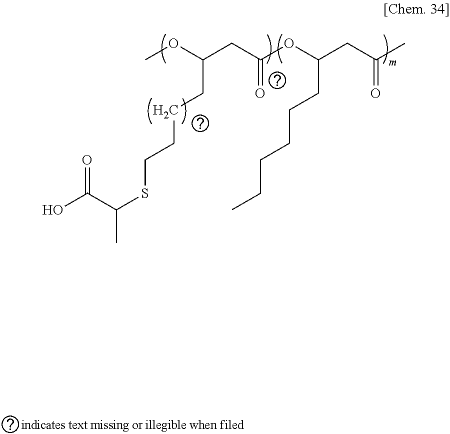

0.1 g of the compound of Example 1″ and 15 mg of thiolactic acid were dissolved in 5 mL of chloroform at room temperature with stirring. 5 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 50 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 11 was fully characterized by spectroscopic and spectrometric methods and is in accordance with the expected chemical structure. Grafting to 100%.

### Example 12: Poly(3-hydroxynonanoate-co-undecenoate) Containing 5% Unsaturations 100% Grafted with Octanethiol

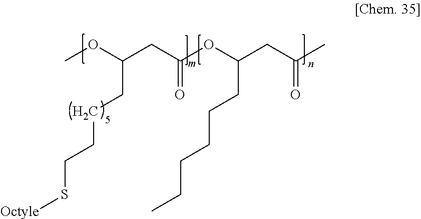

1 g of the PHA copolymer of Example 1′ and 150 mg of octanethiol were dissolved in 15 mL of ethyl acetate at room temperature with stirring. 20 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 500 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The grafted PHA of Example 12 was fully characterized by spectroscopic and spectrometric methods and is in accordance with the expected chemical structure. Grafting to 100%.

### Example 13: Poly(3-hydroxynonanoate-co-undecenoate) Containing 5% Unsaturations 100% Epoxidized

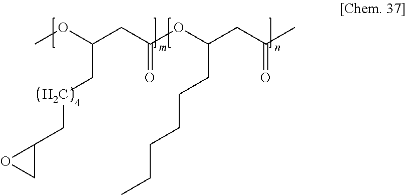

20 g of the PHA copolymer of Example 1′ were dissolved in 80 mL of anhydrous dichloromethane. A suspension of 1.9 g of 77% m-CPBA was prepared with 20 mL of anhydrous dichloromethane and added to the mixture with stirring, at room temperature for at least 120 hours.

The reaction medium was then precipitated from a 500 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The PHA of Example 13 was fully characterized by spectroscopic and spectrometric methods and is in accordance with the expected chemical structure. Epoxidation to 100%.

### Example 14: Poly(3-hydroxynonanoate-co-undecenoate) Containing 10% Unsaturations 100% Epoxidized

10 g of the PHA copolymer of Example 1″ were dissolved in 40 mL of anhydrous dichloromethane. A suspension of 1.9 g of 77% m-CPBA was prepared with 10 mL of anhydrous dichloromethane and added to the mixture with stirring, at room temperature for at least 120 hours.

The reaction medium was then precipitated from a 500 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The PHA of Example 14 was fully characterized by spectroscopic and spectrometric methods and is in accordance with the expected chemical structure. Epoxidation to 100%.

### Example 15: Poly(3-hydroxyoctanoate-co-undecenoate) Containing 30% Unsaturations 100% Epoxidized

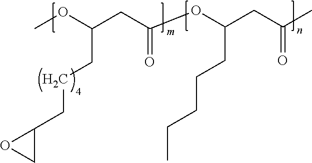

10 g of the PHA copolymer of Example 1′″ were dissolved in 40 mL of anhydrous dichloromethane. A suspension of 6.2 g of 77% m-CPBA was prepared with 10 mL of anhydrous dichloromethane and added to the mixture with stirring, at room temperature for at least 120 hours.

The reaction medium was then precipitated from a 250 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The PHA of Example 15 was fully characterized by spectroscopic and spectrometric methods and is in accordance with the expected chemical structure. Epoxidation to 100%.

### Example 16: Poly(3-Hydroxynonanoate-co-Undecenoate) Containing 5% Unsaturations 100% Grafted with 4-Tert-Butylbenzyl Mercaptan (TBM) (Compound of Example 1′ Grafted with TBM)

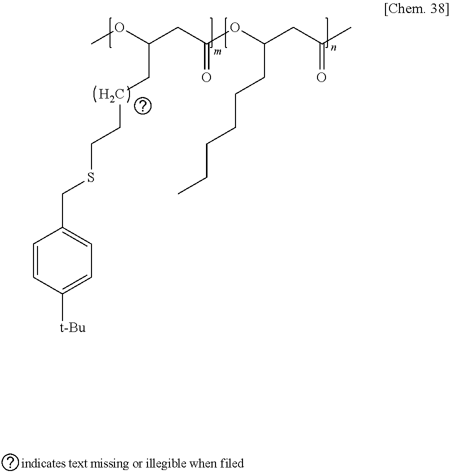

2 g of the PHA copolymer of Example 1′ and 300 mg of 4-tert-butylbenzyl mercaptan were dissolved in 25 mL of ethyl acetate at room temperature with stirring. 25 mg of 2,2-dimethoxy-2-phenylacetophenone (Irgacure 651) were added to the mixture. The medium was then irradiated under a 100 W UV lamp at 365 nm (reference) and with stirring for at least 10 minutes.

The reaction medium was then precipitated from a 500 mL mixture of 70/30 v/v ethanol/water. A viscous white precipitate was obtained. This step may be repeated. The product thus obtained was dissolved in a minimum amount of ethyl acetate, poured onto a Teflon plate and then dried under dynamic vacuum at 40° C. to obtain a homogeneous film.

The PHA of Example 16 was fully characterized by spectroscopic and spectrometric methods and is in accordance with the expected chemical structure. Grafting to 100%.

### Example 17: Copolymer of PHA Bearing a Side Chain R¹ Representing an Isohexenyl Group and R² Representing an Isobutyl Group

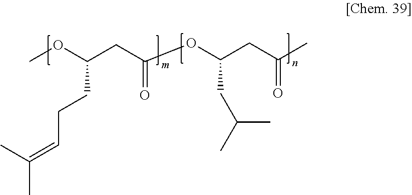

The production process of Example 17 is an adaptation of Applied and Environmental Microbiology, Vol. 60, No. 9. 3245-3254 (1994) “Polyester Biosynthesis Characteristics of Pseudomonas citronellolis Grown on Various Carbon Sources, Including 3-Methyl-Branched Substrate”. Mun Hwan Choi and Sung Chul Yoon. The microorganism used is Pseudomonas citronellolis ATCC® 13674™. The culture method was performed under axenic conditions in unfed batch culture mode in 5 L Fernbach flasks (Corning® ref. 431685) containing 2 of culture medium, shaken at 110 rpm at 30° C. in an orbital incubator (orbit diameter of 2.5 cm).

The production process is performed using two different culture media. The first culture medium, defined CM1 “inoculum”, is used for the preparation of the preculture. The second culture medium, defined CM2 “batch”, is used for unfed batch culture growth of the microorganism with the carbon source of interest in the Fernbach flasks.

TABLE 6

| Ingredients in grams per litre | CM1 <<inoculum>> | CM2 <<batch>> |
|---|---|---|
| (NH4)₂SO₄ | / | 0.66 |
| Na₂HPO₄•7H₂O | / | 7.3 |
| KH₂PO4 | / | 2.3 |
| NaHCO₃ | / | 0.3 |
| CaCl₂•2H₂O | / | 0.1 |
| MgSO₄•7H₂O | / | 0.25 |
| Citric acid | / | 1.03 |
| Citronellol | / | 5.5 |
| Microelement solution | / | 1 |
| Nutrient broth | 1.5 | / |
| Yeast extract | 1 | / |

| | |
|---|---|
| 2N NaOH | qs pH = 6.8 |
| MilliQ water | qs m = 1000 g |

The composition of the Nutrient Broth, as mass percentages, is 37.5% beef extract and 62.5% peptone. Reference 233000 DIFCO™ BD.

The composition of the yeast extract, as a mass percentage, is 100% autolysate of the yeast Saccharomyces cerevisiae. Reference 210933 DIFCO™ BD.

TABLE 7

| Ingredients in grams per litre | Amount | |
|---|---|---|
| FeSO₄•7H₂O | 5.56 | g |
| CaCl₂•2H₂O | 3.0 | g |
| ZnSO₄•7H₂O | 0.58 | g |
| MnCl₂•4H₂O | 3.86 | g |
| H₃BO₃ | 0.6 | g |
| CoCl₂•6H₂O | 5.62 | g |
| Na₂MoO₄•2H₂O | 0.06 | g |
| NiCl₂•6H₂O | 0.04 | g |
| CuSO₄•5H₂O | 0.34 | g |

| | |
|---|---|
| 0.5N HCl | qs 1000 g |

100 mL of preculture are prepared by suspending a cryotube containing 1 mL of the strain with 100 mL of “inoculum” culture medium at a pH adjusted to 6.8 with 2N NaOH in a 250 mL Fernbach flask and then incubated at 30° C. at 150 rpm for 24 hours. 1.9 L of CM2 “batch” culture medium placed in a presterilized 5 L Fernbach flask are inoculated at O_{D}=0.1 with 100 mL of inoculum.

After 70 hours at 30° C. at 110 rpm, the biomass is dried by lyophilization before being extracted with dichloromethane for 24 hours. The suspension is clarified by filtration on a GF/A filter (Whatman®). The filtrate, composed of PHA dissolved in dichloromethane, is concentrated by evaporation and then dried under high vacuum at 40° C. to constant mass.

The PHA may optionally be purified by successive dissolution and precipitation, for instance using a dichloromethane/methanol system.

The PHA copolymer of Example 17 was fully characterized by spectroscopic and spectrometric methods and is in accordance with the expected chemical structure, with: 68 mol % of unit (A) for which R¹=isohexenyl and 32 mol % of unit (B) for which R²=isobutyl.

### Example 18: Copolymer of PHA Bearing a Side Chain R¹ Representing an Isohexyl Group and R² Representing an Isobutyl Group

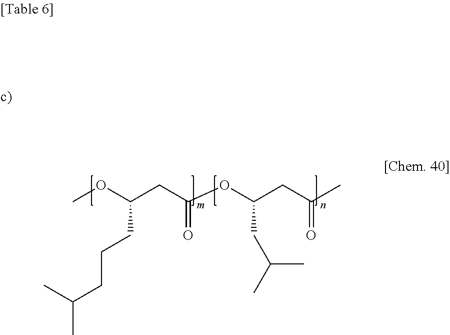

Example 18 is obtained by hydrogenation of the PHA copolymer of Example 17 using an H-Cube Midi® continuous hydrogenator from ThalesNano Technologies.

A solution of 2 g (8.83 mmol) of PHA of Example 3 is prepared with a mixture composed of 100 ml of ethyl acetate (Sigma-Aldrich—CAS: 141-78-6) and 100 mL of methanol (Sigma-Aldrich—CAS: 67-56-1) and is introduced at a flow rate of 3 mL per minute into a hydrogenation cartridge containing the catalyst containing 5% palladium on charcoal (MidiCard ref. DHS 2141; ThalesNano Technologies) maintained at 100° C. under a pressure of 80 bar in the presence of hydrogen in the ThalesNano Technologies H-Cube Midi® system. The reduction of the double bond is monitored by NMR. After six consecutive cycles of reduction, the solution is concentrated by evaporation and then dried under vacuum to constant mass.

The PHA may optionally be purified by successive dissolution and precipitation, for instance using a dichloromethane/methanol system.

The PHA copolymer of Example 4 was fully characterized by spectroscopic and spectrometric methods and is in accordance with the expected chemical structure, with: 68 mol % of unit (A) for which R¹=isohexyl and 32 mol % of unit (B) for which R²=isobutyl.

### Example 19

A polymer was prepared using the microorganism Pseudomonas putida KT2440 ATCC® 47054™ and octanoic acid.

The culture method was performed under batch axenic conditions in 5 L Fernbach flasks (Corning® ref. 431685) containing 2 L of culture medium, shaken at 110 rpm at 30° C. in an orbital incubator (orbit diameter of 2.5 cm). The synthetic process was performed using two different culture media. The first culture medium, defined CM1 “inoculum”, was used for the preparation of the inoculum. The second culture medium, defined CM2 “batch”, was used for unfed batch growth of the microorganism with the octanoic acid in the Fernbach flasks.

The composition in grams per litre of the two media is described in Table 8 below:

TABLE 8

| | CM1 | CM2 |
|---|---|---|
| | « inoculum » | « batch » |
| (NH₄)₂SO₄ | 4.7 | 5.02 |
| Na₂HPO₄•7H₂O | 12 | 2.24 |
| KH₂PO₄ | 2.7 | 0.5 |
| Glucose | 9 | 3.9 |
| MgSO₄•7H₂O | 0.8 | 1.03 |
| Citric acid | 1.6 | 1.03 |
| Nutrient broth (1) | 1 | / |
| Octanoic acid | / | 3.8 |
| Microelements solution (2) | / | 1.4 |

| | |
|---|---|
| 2N NaOH | qs pH = 6.8 |
| Water | qs 1000 g |

(1) The composition of the Nutrient Broth, as mass percentages, is 37.5% beef extract and 62.5% peptone. Reference 233000 DIFCO ™.
(2) The composition of the microelement solution in grams per litre is described in Table 9 below:

TABLE 9

| | | |
|---|---|---|
| FeSO₄•7H₂O | 10.0 | g |
| CaCl₂•2H₂O | 3.0 | g |
| ZnSO₄•7H₂O | 2.2 | g |
| MnSO₄•4H₂O | 0.5 | g |
| H₃BO₃ | 0.3 | g |
| CoCl₂•6H₂O | 0.2 | g |
| Na₂MoO₄•2H₂O | 0.15 | g |
| NiCl₂•6H₂O | 0.02 | g |
| CuSO₄•5H₂O | 1.00 | g |

| | |
|---|---|
| 0.5 N HCl | qs 1000 g |

100 ml of inoculum were prepared by suspending a cryotube containing 1 ml of the strain with 100 ml of “inoculum” culture medium at a pH adjusted to 6.8 with 2N NaOH in a 250 mL Fernbach flask and then incubated at 30° C. at 150 rpm for 24 hours. 1.9 L of CM2 “batch” culture medium placed in a presterilized 5 L Fernbach flask were inoculated at O_{D}=0.1 with 100 mL of inoculum. After 70 hours at 30° C. at 110 rev/min, the biomass was dried by lyophilization before being extracted with dichloromethane for 24 h. The suspension was clarified by filtration on a GF/A filter (Whatman®). The filtrate, containing the copolymer in solution in the dichloromethane, was concentrated by evaporation and then dried under high vacuum at 40° C. to constant weight. The crude polyhydroxyalkanoate was purified by precipitation of a solution of the latter in solution in 10 times its weight of dichloromethane from 10 volumes of the solution of cold methanol. The solid obtained was dried under high vacuum at 40° C. to constant weight.

The molecular weight of the polyhydroxyalkanoate obtained was characterized by size exclusion chromatography, with detection by refractive index.
Eluent: THF
Analytical flow rate: 1 mL/min
Injection: 100 μL
Columns: 1 Agilent PLGeI Mixed-D 5 μm column; 300×7.5 mm; 1 Agilent PLGeI Mixed-C 5 μm column; 300×7.5 mm; 1 Agilent Oligopore column; 300×7.5 mm
at room temperature (25° C.)
Detection: Waters 2487 Dual I Absorbance Detector, Waters 2414 Refractive Index Detector
Integrator: refractive index at 45° C. and 64 mV
Empower (GC Relative molar mass/conventional module)
Empower injection time: 40 min
Standards: High mass/EasiVial PS-H 4 mL polystyrene from Agilent Technologies, Part No. PL2010-0200

The analysis makes it possible to measure the weight-average molecular weight (Mw in g/mol), the number-average molecular weight (Mn in g/mol), the polydispersity index PI (Mw/Mn) and the degree of polymerization DPn.

The monomeric composition of the polyhydroxyalkanoate obtained was defined by gas chromatography equipped with a flame ionization detector. The identification is performed by injection of commercial standards and the monomer composition was determined by a methanolysis and silylation treatment. To determine the monomer composition, 7 mg of the polyhydroxyalkanoate polymer were dissolved in 1.5 mL of chloroform and subjected to methanolysis in the presence of 1.5 mL of an MeOH/HCl solution (17/2, v/v) at 100° C. for 4 hours. The organic phase was then washed with 1 mL of water and then dried over MgSO₄. Silylation of the methyl esters formed was performed by adding 100 μL of BSTFA (N,O-bis(trimethylsilyl)trifluoroacetamide) and 100 μL of pyridine to the methylated sample. The solution was heated at 70° C. for 1 hour and then evaporated to dryness. The sample is then dissolved in 600 μL of dichloromethane and analysed by chromatography under the following conditions:
Hewlett Packard 6890 Series machine
ZB-5 HT stationary phase column from Phenomenex (length: 30 m, diameter: 0.25 mm)
Temperature: isotherm 60° C. to 300° C. in 6 min (heating rate: 10° C./min)
Gas: Helium; flow rate: 0.8 mL/min
Injector: Temperature: 250° C.; 50 ml/min
Flame ionization detector; Temperature: 300° C.
Injection: Volume 1 μL

A copolymer containing 91% by weight of poly(3-hydroxyoctanoate), 6% by weight of poly(3-hydroxyhexanoate) and 3% by weight of poly(3-hydroxybutanoate) was thus obtained.

$$Mn = 68 \; 100 g / mol$$

$$Mw = 149 \; 100 g / mol$$

$$Ip = 2.2$$

$$DPn = 531$$

### Example 20

A polymer was prepared using the microorganism Pseudomonas putida KT2440 ATCC® 47054™, octanoic acid and acrylic acid.

The culture method was performed under continuous axenic conditions at a dilution D=0.25 h⁻¹ in a 3 L chemostat containing 1.1 L of culture medium. The system was aerated with air at a flow of 3 vvm (vvm=volume of air per volume of fermentation medium per minute) for a nominal dissolved oxygen (O_{D}) value at 30% of saturation.

The production process was performed using three different culture media. The first undefined culture medium (CM1) was used for the preparation of the inoculum. The second defined culture medium (CM2) was used for the unfed batch growth of the microorganism in the fermenter. The third defined culture medium (CM3) was used for the feeding, or maintenance, of the continuous fermentation containing octanoic acid and acrylic acid (inhibitor of the β-oxidation pathway). The CM1 and CM2 media are identical to those described in example 1. The composition in grams per litre of the medium CM3 is described in Table 10 below:

TABLE 10

| | CM3 « continuous » |
|---|---|
| (NH₄)₂SO₄ | 5.02 |
| Na₂HPO₄•7H₂O | 2.24 |
| KH₂PO₄ | 0.5 |
| Glucose | 3 |
| MgSO₄•7H₂O | 1.03 |
| Citric acid | 1.03 |
| Nutrient Broth (1) | / |
| Octanoic acid | 3.8 |
| Microelements solution (2) | 1.4 |
| Acrylic acid | 0.2 |
| 2N NaOH | QSP pH = 6.8 |
| Water | gs 1000 g |

100 ml of inoculum were prepared by suspending a cryotube containing 1 ml of the strain with 100 ml of Nutrient Broth at a pH adjusted to 7.0 with 2N NaOH in a 250 ml Fernbach flask and were then incubated at 30° C. at 150 rev/min for 24 h.

The fermenter containing 1 litre of culture medium CM2 at 30° C. was inoculated at an optical density of 0.1 at 630 nm (O_{D 630}=0.1). The system was maintained at 30° C. with shaking at 700±200 rpm and regulated in cascade with oxygenation for about 16 hours and/or the time for the microorganism to be able to reach its growth plateau.

Feeding of the fermenter with the medium CM3 was initiated when the microorganism reached its growth plateau, and withdrawal was then performed so as to maintain the initial mass of fermentation medium. Once the equilibrium state was reached in continuous culture, a fraction of the withdrawn material was centrifuged in order to separate the biomass from the fermentation medium. The biomass was dried by lyophilization and then extracted with dichloromethane for 24 hours. The suspension obtained was clarified by filtration through a GF/A filter (Whatman®). The filtrate obtained, comprising the copolymer dissolved in dichloromethane, was concentrated by evaporation and then dried under high vacuum at 40° C. to constant mass. The crude polyhydroxyalkanoate was purified by precipitation of a solution of the latter in solution in 10 times its weight of dichloromethane from 10 volumes of the solution of cold methanol. The solid obtained was dried under high vacuum at 40° C. to constant weight. A copolymer comprising 96% by weight of poly(3-hydroxyoctanoate), 3% by weight of poly(3-hydroxyhexanoate) and 1% by weight of poly(3-hydroxybutanoate) was thus obtained.

$$Mn = 67 \; 900 g / mol :$$

$$Mw = 142 \; 000 g / mol :$$

$$Ip = 2.1 :$$

$$DPn = 611$$

### Example 21: Copolymer of PHA Bearing a Side Chain R¹ Representing an n-Hexyl Group and R² Representing an n-Butyl Group

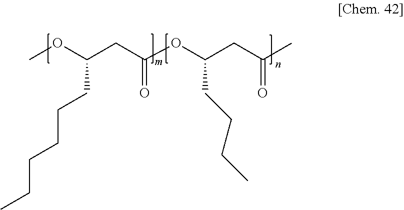

A polymer was prepared using the microorganism Pseudomonas putida KT2440 ATCC® 47054™, nonanoic acid and acrylic acid. The culture method is performed under continuous axenic conditions at a dilution D=0.25 h⁻¹ in a 3 L chemostat containing 1.1 L of culture medium. The system is aerated with a flow of 1 vvm of air for a nominal dissolved oxygen (O_{D}) value at 30% of saturation.

The production process is performed using three different culture media. The first culture medium (CM1) is used for the preparation of the inoculum. The second culture medium (CM2) is used for batch growth of the microorganism in the fermenter. The third culture medium (CM3) is used for the feeding, or maintenance, of the continuous fermentation containing the carbon source of interest and the β-oxidation pathway inhibitor (acrylic acid). The composition in grams per litre of the three media CM1, CM2 and CM3 is described in Table 11 below:

TABLE 11

| | CM1 | CM2 | CM3 |
|---|---|---|---|
| | “inoculum” | “batch” | “continuous” |
| (NH₄)₂SO₄ | 4.7 | 5.02 | 5.02 |
| Na₂HPO₄•7H₂O | 12 | 2.24 | 2.24 |
| KH₂PO₄ | 2.7 | 0.5 | 0.5 |
| Glucose | 9 | 3.9 | 3.9 |
| MgSO₄•7H₂O | 0.8 | 1.03 | 1.03 |
| Citric acid | 1.6 | 1.03 | 1.03 |
| Nutrient broth | 1 | / | / |
| Nonanoic acid | / | / | 3.8 |
| Microelement solution | / | 1.4 | 1.4 |
| Acrylic acid | / | / | 0.2 |

| | |
|---|---|
| 2N NaOH | qs pH = 6.8 |
| MilliQ water | qs m = 1000 g |

The composition of the Nutrient Broth, as mass percentages, is 37.5% beef extract and 62.5% peptone. Reference 233000 DIFCO™.

The composition of the microelement solution in grams per litre is described in Table 12 below.

TABLE 12

| | | |
|---|---|---|
| FeSO₄•7H₂O | 10.0 | g |
| CaCl₂•2H₂O | 3.0 | g |
| ZnSO₄•7H₂O | 2.2 | g |
| MnSO₄•4H₂O | 0.5 | g |
| H₃BO₃ | 0.3 | g |
| CoCl₂•6H₂O | 0.2 | g |
| Na₂MoO₄•2H₂O | 0.15 | g |
| NiCl₂•6H₂O | 0.02 | g |
| CuSO₄•5H₂O | 1.00 | g |

| | |
|---|---|
| MilliQ water | qs 1000 g |

100 mL of inoculum are prepared by suspending a cryotube containing 1 mL of the strain at O_{D}=10 with 100 mL of CM1 “inoculum” at a pH preadjusted to 7.0 with 2N NaOH in a 500 mL Fernbach flask and are then incubated at 30° C. at 150 rpm for 24 hours.

The 3 L fermenter containing 1 litre of CM2 “batch” culture medium at 30° C. is inoculated at an optical density of 0.1 at 600 nm (O_{D 600}=0.1). The system is maintained at 30° C. with shaking at 700±200 rpm and regulated in cascade with oxygenation for about 16 hours and/or the time for the microorganism to be able to reach its growth plateau.

Feeding of the fermenter with the CM3 “continuous” medium is initiated when the microorganism has reached its growth plateau, and withdrawal is then performed so as to maintain the initial mass of fermentation medium. Once the equilibrium state is reached in continuous culturing, a fraction of the withdrawn material is centrifuged so as to separate the biomass from the fermentation medium. The biomass is dried by lyophilization and is then extracted with dichloromethane for 24 hours. The suspension is clarified by filtration on a GF/A filter (Whatman®). The filtrate, composed of PHA dissolved in dichloromethane, is concentrated by evaporation and then dried under high vacuum at 40° C. to constant mass.

The molecular weight of the polyhydroxyalkanoate obtained was characterized by size exclusion chromatography, with detection by refractive index.
Eluent: THF
Analytical flow rate: 1 mL/min
Injection: 100 μL
Columns: 1 Agilent PLGeI Mixed-D 5 μm column; 300×7.5 mm; 1 Agilent PLGeI Mixed-C 5 μm column; 300×7.5 mm; 1 Agilent Oligopore column; 300×7.5 mm
at room temperature (25° C.)
Detection: Waters 2487 Dual I Absorbance Detector, Waters 2414 Refractive Index Detector
Integrator: refractive index at 45° C. and 64 mV
Empower (GC Relative molar mass/conventional module)
Empower injection time: 40 min
Standards: High mass/EasiVial PS-H 4 mL polystyrene from Agilent Technologies, Part No. PL2010-0200

The analysis makes it possible to measure the weight-average molecular weight (Mw in g/mol), the number-average molecular weight (Mn in g/mol), the polydispersity index PI (Mw/Mn) and the degree of polymerization DPn.

The monomeric composition of the polyhydroxyalkanoate obtained was defined by gas chromatography equipped with a flame ionization detector. The identification is performed by injection of commercial standards and the monomer composition was determined by a methanolysis and silylation treatment. To determine the monomer composition, 7 mg of the polyhydroxyalkanoate polymer were dissolved in 1.5 mL of chloroform and subjected to methanolysis in the presence of 1.5 mL of an MeOH/HCl solution (17/2, v/v) at 100° C. for 4 hours. The organic phase was then washed with 1 mL of water and then dried over MgSO₄. Silylation of the methyl esters formed was performed by adding 100 μL of BSTFA (N,O-bis(trimethylsilyl)trifluoroacetamide) and 100 μL of pyridine to the methylated sample. The solution was heated at 70° C. for 1 hour and then evaporated to dryness. The sample is then dissolved in 600 μL of dichloromethane and analysed by chromatography under the following conditions:
Hewlett Packard 6890 Series machine
ZB-5 HT stationary phase column from Phenomenex (length: 30 m, diameter: 0.25 mm)
Temperature: isotherm 60° C. to 300° C. in 6 min (heating rate: 10° C./min)
Gas: Helium; flow rate: 0.8 ml/min
Injector: Temperature: 250° C.; 50 ml/min
Flame ionization detector; Temperature: 300° C.
Injection: Volume 1 μL

A copolymer comprising 86% by weight of poly(3-hydroxynonanoate), 9% by weight of poly(3-hydroxyheptanoate) and 5% by weight of poly(3-hydroxypentanoate) was thus obtained.

$$Mn = 65 \; 900 g / mol$$

$$Mw = 143 \; 600 g / mol$$

$$Ip = 2.2$$

$$DPn = 531$$

### Example 22

A polymer was prepared according to the procedure of example 19 using nonanoic acid (instead of octanoic acid) and without acrylic acid.

A copolymer comprising 68% by weight of poly(3-hydroxynonanoate), 27% by weight of poly(3-hydroxyheptanoate) and 5% by weight of poly(3-hydroxypentanoate) was thus obtained.

$$Mn = 55 \; 800 g / mol$$

$$Mw = 124 \; 500 g / mol$$

$$Ip = 2.2$$

$$DPn = 469$$

### Example 23

A polymer was prepared according to the procedure of example 19 using dodecanoic acid (instead of octanoic acid).

A copolymer comprising 44% by weight of poly(3-hydroxydodecanoate), 38% by weight of poly(3-hydroxydecanoate) and 18% by weight of poly(3-hydroxyoctanoate) was thus obtained.

$$Mn = 67 \; 400 g / mol$$

$$Mw = 129 \; 800 g / mol$$

$$Ip = 1.9$$

$$DPn = 484$$

### Example 24: Copolymer of PHA Bearing a Side Chain R¹ Representing an n-Pentyl Group and R² Representing an n-Propyl Group

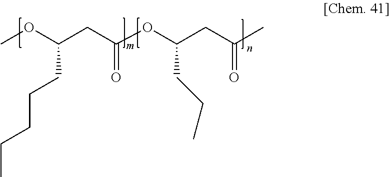

The production process of Example 24 is an adaptation of the article Biomacromolecules 2012, 13, 2926-2932: “Biosynthesis and Properties of Medium-Chain-Length Polyhydroxyalkanoates with Enriched Content of the Dominant Monomer”

The microorganism used is Pseudomonas putida ATCC® 47054™.

The culture method is performed under continuous axenic conditions at a dilution D=0.25 h⁻¹ in a 3 L chemostat containing 1.1 L of culture medium.

The system is aerated with a flow of 3 vvm of air for a nominal dissolved oxygen (O_{D}) value at 30% of saturation.

### Assembly:

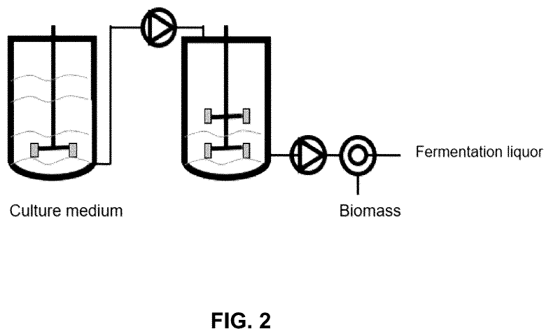

See FIG. 2

The production process is performed using three different culture media.

The first undefined culture medium (CM1) is used for the preparation of the inoculum.

The second defined culture medium (CM2) is used for batch growth of the microorganism in the fermenter.

The third defined culture medium (CM3) is used for the feeding, or maintenance, of the continuous fermentation containing the carbon source of interest and the pi-oxidation pathway inhibitor.

The composition in grams per litre of the three media is described in Table 13. Composition in grams per litre of the culture media for the inoculum and for maintenance.

TABLE 13

| | CM1 | CM2 | CM3 |
|---|---|---|---|
| | « inoculum » | « batch » | « continuous » |
| (NH4)₂SO4 | 4.7 | 5.02 | 5.02 |
| Na₂HPO4•7H2O | 12 | 2.24 | 2.24 |
| KH₂PO4 | 2.7 | 0.5 | 0.5 |
| Glucose | 9 | 3.9 | 3 |
| MgSO4•7H2O | 0.8 | 1.03 | 1.03 |
| Citric acid | 1.6 | 1.03 | 1.03 |
| Nutrient broth | 1 | / | / |
| Octanoic acid | / | / | 3.8 |
| Microelements solution | / | 1.4 | 1.4 |
| Acrylic acid | / | / | 0.2 |

| | |
|---|---|
| 2N NaOH | qs pH = 6.8 |
| MilliQ water | qs m = 1000 g |

The composition of the Nutrient Broth, as mass percentages, is 37.5% beef extract and 62.5% peptone. Reference 233000 DIFCO™.

The composition of the microelement solution in grams per litre is described in Table 14: composition in grams per litre of the microelement solution

TABLE 14

| | | |
|---|---|---|
| FeSO4•7H2O | 10.0 | g |
| CaCl2•2H2O | 3.0 | g |
| ZnSO4•7H2O | 2.2 | g |
| MnSO4•4H2O | 0.5 | g |
| H3BO3 | 0.3 | g |
| CoCl2•6H2O | 0.2 | g |
| Na2MoO4•2H2O | 0.15 | g |
| NiCl2•6H2O | 0.02 | g |
| CuSO4•5H2O | 1.00 | g |

| | |
|---|---|
| MilliQ water | qs 1000 g |

100 mL of inoculum are prepared by suspending a cryotube containing 1 mL of the strain with 100 mL of Nutrient Broth at a pH adjusted to 7.0 with 2N NaOH in a 250 mL Fernbach flask and are then incubated at 30° C. at 150 rpm for 24 hours.

The fermenter containing 1 litre of culture medium CM2 at 30° C. was inoculated at an optical density of 0.1 at 630 nm (O_{D 630}=0.1). The system is maintained at 30° C. with shaking at 700±200 rpm and regulated in cascade with oxygenation for about 16 hours and/or the time for the microorganism to be able to reach its growth plateau.

Feeding of the fermenter with the medium CM3 is initiated when the microorganism has reached its growth plateau, and withdrawal is then performed so as to maintain the initial mass of fermentation medium. Once the equilibrium state is reached in continuous culturing, a fraction of the withdrawn material is centrifuged so as to separate the biomass from the fermentation medium. The biomass is dried by lyophilization and is then extracted with dichloromethane for 24 hours. The suspension is clarified by filtration on a GF/A filter (Whatman®). The filtrate, composed of PHA dissolved in dichloromethane, is concentrated by evaporation and then dried under high vacuum at 40° C. to constant mass.

The PHA may optionally be purified by successive dissolution and precipitation, for instance using a dichloromethane/methanol system.

The PHA copolymer of Example 24 was fully characterized by spectrometric and spectroscopic methods. By gas chromatography equipped with an FID detector, it is seen that the copolymer contains 96% of radical R¹=n-pentyl and 4% of radical R²=n-propyl.

### Example 25: PHA Bearing a Side Chain R¹ Representing a Linear 5% Unsaturated 8-Bromo-n-Octanoyl Group and R² Representing an n-Hexyl Group

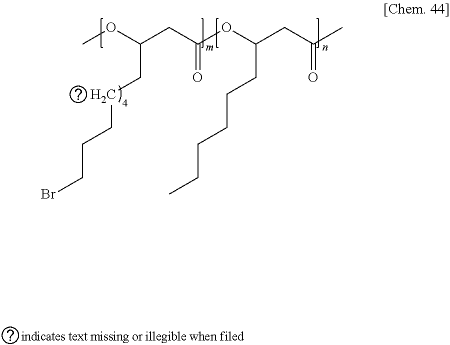

The process for synthesizing the compound of Example 1 is adapted from the article: Fed-batch production of unsaturated medium-chain-length polyhydroxyalkanoates with controlled composition by Pseudomonas putida KT2440, Z. Sun, J. A. Ramsay, M. Guay, B. A. Ramsay, Applied Microbiology Biotechnology, 82. 657-662, 2009.

The microorganism used is Pseudomonas putida KT2440 ATCC® 47054™. The culture method is performed under fed-batch growth axenic conditions with a maintenance solution containing a mixture of carbon source at a rate μ=0.15 h⁻¹ in a 3 L chemostat containing 2.5 L of culture medium.

The system is aerated with a flow of 0.5 vvm of air for a nominal dissolved oxygen (O_{D}) value at 30% of saturation. The pH is regulated with a solution composed of ammonia and glucose at 15% and 40% final mass, respectively. The temperature of the fermentation medium is regulated at 30° C.

Equipment for the fed-batch growth fermentation mode:

The fermentation medium is regulated in terms of temperature-pressure of dissolved oxygen and pH (not shown).

The production process is performed using three different culture media. The first culture medium, defined CM1 “inoculum”, is used for the preparation of the preculture. The second culture medium, defined CM2 “batch”, is used for unfed batch growth of the microorganism with the primary carbon sources in the Fernbach flasks. The third culture medium, defined CM3 “maintenance”, is used for the fed-batch or maintenance fermentation mode with the carbon sources of interest at a flow rate calibrated as a function of the growth of the microorganism.

TABLE 15

| Ingredients in grams | CM1 | CM2 | CM3 |
|---|---|---|---|
| per litre | <<inoculum>> | <<batch>> | <<maintenance>> |
| (NH₄)₂SO₄ | 4.7 | 4.7 | |
| Na₂HPO₄•7H₂O | 12 | 9 | |
| KH₂PO₄ | 2.7 | 2.03 | |
| MgSO₄•7H₂O | 0.8 | 1.03 | |
| Nutrient broth | 3 | / | |
| Nonanoic acid | / | 1 | 923 |
| 11-Bromoundecanoic | / | 0 | 77 |
| acid | | | |
| Microelements solution | / | 10 | |

| | |
|---|---|
| 2N NaOH | qs pH = 6.8 |
| MilliQ water | qs 1000 g |

The composition of the Nutrient Broth, as mass percentages, is 37.5% beef extract and 62.5% peptone. Reference 233000 DIFCO™.

TABLE 16

| Ingredients in grams per litre | Amount | |
|---|---|---|
| FeSO₄•7H₂O | 10.0 | g |
| CaCl₂•2H₂O | 3.0 | g |
| ZnSO₄•7H₂O | 2.2 | g |
| MnSO₄•4H₂O | 0.5 | g |
| H₃BO₃ | 0.3 | g |
| CoCl₂•6H₂O | 0.2 | g |
| Na₂MoO₄•2H₂O | 0.15 | g |
| NiCl₂•6H₂O | 0.02 | g |
| CuSO₄•5H₂O | 1.00 | g |

| | |
|---|---|
| MilliQ water (or 0.5N HCl) | qs 1000 g |

100 mL of preculture are prepared by suspending a cryotube containing 1 mL of the strain with 100 mL of “inoculum” culture medium at a pH adjusted to 6.8 with 2N NaOH in a 250 mL Fernbach flask and then incubated at 30° C. at 150 rpm for 24 hours. 1.9 L of CM2 “batch” culture medium placed in a presterilized 3 L chemostat are inoculated at O_{D}=0.1 with 100 mL of preculture. After 4 hours at 30° C. at 850 rpm, introduction of the maintenance culture medium is performed, applying the flow rate defined by equation 1.

At the end of the introduction, the biomass is isolated by centrifugation and then washed three times with water. The biomass is dried by lyophilization before being extracted with ethyl acetate for 24 hours. The suspension is clarified by filtration on a GF/A filter (Whatman®). The filtrate, composed of PHA dissolved in the ethyl acetate, is concentrated by evaporation and then dried under high vacuum at 40° C. to constant mass.

The PHA may optionally be purified by dissolution in ethyl acetate and successive precipitations from a 70/30 v/v ethanol/water system, for example.

The PHA was fully characterized by spectroscopic and spectrometric methods and is in accordance with the expected chemical structure: 95 mol % of unit (B) for which R²=n-hexyl (71%) and n-butyl (24%) and 5 mol % of unit (A) for which R¹=8-bromo-n-octanyl (5.9%) and 6-bromo-n-hexyl (0.2%).

Compounds a) of Examples 1 to 25 may be mixed with one or more crystallizable fatty substances b) as defined previously, in the presence of one or more liquid oils c) such as isododecane and optionally water e). The mixing of the PHA(s) a) with the crystallizable fatty substance(s) b) may be performed at room temperature, with stirring, preferably in the presence of a liquid fatty substance c) and optionally of organic solvent(s) other than c) or d) as defined previously. According to one variant, water e) is added to the mixture of a), b) and c) and one or more organic solvents other than c) and d) as defined previously are then optionally added.

### Examples 26 to 34

Compositions 26 (comparative) and 27 to 34 (invention) described in Table 17 below were prepared:

TABLE 17

| Ingredients* | Ex. 26 | Ex. 27 | Ex. 28 | Ex. 29 |
|---|---|---|---|---|
| PHA of Example 21 | 20 | 20 | 20 | 20 |
| Trihydroxystearin | — | 5 | — | — |
| Syncrowax HRC-PA: tribehenin | — | — | 5 | — |
| Compritol 888 CG ATO: glyceryl | — | — | — | 5 |
| dibehenate and tribehenin and | | | | |
| glyceryl behenate | | | | |
| Isododecane | 77.5 | 72.5 | 72.5 | 72.5 |
| Ethanol | 2.5 | 2.5 | 2.5 | 2.5 |
| Ingredients* | Ex. 30 | Ex. 31 | Ex. 32 | Ex. 33 |
| PHA of Example 21 | 20 | 20 | 20 | 20 |
| Refined sunflower oil: | 5 | — | — | — |
| Helianthus annuus sunflower | | | | |
| seed wax | | | | |
| (C₂₀-C₆₀) microcrystalline wax | — | 5 | — | — |
| C₁₈-C₃₈ fatty alcohol | — | — | 5 | — |
| hydroxystearoyl stearate: | | | | |
| synthetic wax | | | | |
| (Synthetic Beeswax) | | | | |
| Candelilla wax: Euphorbia | — | — | — | 5 |
| cerifera (candelilla) wax | | | | |
| Isododecane | 72.5 | 72.5 | 72.5 | 72.5 |
| Ethanol | 2.5 | 2.5 | 2.5 | 2.5 |

*Data by weight in g

### Composition Preparation Procedure:

The PHA, isododecane and ethanol are stirred at 2500 rpm, at a temperature of 25° C. The crystallizable fatty substance is introduced and the medium is heated from 25° C. to 80° C. with stirring at 2500 rpm. The medium is maintained at 80° C. for 30 minutes with stirring at 3000 rpm and is then cooled from 80° C. to 25° C. with stirring at 2500 rpm.

### Performance Evaluations

### Wear Resistance

### Description of the Test:

The first step in this test consists in making a deposit. The deposits are prepared on a Byko Chart Lenata contrast card with a film spreader and left to dry for 24 hours at 25° C. and 45% RH. The final thickness of the deposit is 30 μm.

A wear resistance test is performed on this dry deposit. A hydrophilic steel ball is used as a friction device. The load or normal force applied is 1 N, and the displacement speed is 50 mm·s⁻¹. On each film are defined tracks on which the friction device makes multiple passes. In the case of wear measurements, permanent contact is maintained during the to and fro trips of the ball on the deposit. The number of passes is increased for each track. The wear resistance is quantified as the minimum number of passes to completely wear out the deposit.

In the case of this study, the number of passes per track are, respectively, 10, 30, 50, 100, 200 and 300 passes.

Each measurement was repeated five times.

The results of the wear resistance tests are quantified as described in the table below:

TABLE 18

| Wear resistance | Evaluations |
|---|---|
| 0-10 passes of the ball | −− |
| 10-30 passes of the ball | − |
| 30-50 passes of the ball | + |
| 50-100 passes of the ball | ++ |
| 100-200 passes of the ball | +++ |
| 200-300 passes of the ball | ++++ |
| >300 passes of the ball | +++++ |

Results:

| Examples | Evaluations |
|---|---|
| 26 (comparative) | −− |
| 27 | ++ |
| 28 | ++ |
| 29 | +++ |
| 30 | +++++ |
| 31 | +++++ |
| 32 | ++ |
| 33 | +++++ |

It is seen that the compositions according to the invention are significantly more resistant than the comparative composition not including any crystallizable fatty substance b).

### Resistance to Water

### Description of the Test:

On the same 30 μm dry deposit made for the wear test, the sensitivity to stressors is evaluated after depositing a drop of stressor (20 μl for water) on the surface of the deposit. The evaluations are made after 1 hour of contact between the stressor and the deposit. The level of sensitivity to stressors is noted as follows. It is seen that all the compositions of the invention (Ex. 27 to 33) are highly resistant to water since the films remained intact.

### Example 1″″: PHA Copolymer Bearing a Side Chain R¹ Representing a 2% Unsaturated n-Octenyl Group and R² Representing an n-Hexyl Group Unsaturated Carried Out in Discontinuous Culture Fed with Two Sources of Carbon in C₉ and C11: 1 98/2

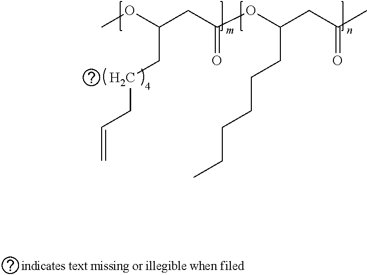

The process for obtaining example 1″ is adapted from Appl Microbiol Biotechnol 82:657-662 (2009) “Fed-batch production of unsaturated medium-chain-length polyhydroxyalkanoates with controlled composition by Pseudomonas putida KT2440”

The microorganism used is Pseudomonas putida KT2440 ATCC® 47054™

The culture mode is carried out under axenic conditions in discontinuous growth fed with a maintenance solution containing a mixture of carbon sources at a rate of μ=0.15 h−1 in a 3 L chemostat containing 2.5 L of medium of culture. The flow rate of the maintenance supply pump is proportional to the growth of the microorganism according to formula 1:

$$St = \frac{{X}_{t}}{{Y}_{X / S}} = \frac{{X}_{0}}{{Y}_{X / S}} {e}^{- t}$$

Formula 1: theoretical equation linking the quantity of biomass and carbon source as a function of time with St=quantity of carbon source required to produce the biomass Xₜ at time t (g), Y_{X/S}=biomass yield from the carbon source, X_{O}=initial biomass (g) and μ=desired specific growth rate (h−1)

The system is aerated by an air flow of 0.5 vvm for a dissolved oxygen (DO) setpoint at 30% saturation. The pH is regulated with a 15% of ammonia solution. The temperature of the fermentation medium is regulated at 30° C. The Assembly of the fed batch growth fermentation mode is made according FIG. 1.

The fermentation medium is regulated in temperature-dissolved oxygen pressure and pH (not shown on the fig.).

The production process is carried out using three distinct culture media. The first culture medium defined MC1 “inoculum” is used for the preparation of the preculture.

The second culture medium defined MC2 “bach” is used for the non-supplied discontinuous growth of the microorganism with the primary carbonaceous sources in the Fernbachs flasks.

The third culture medium defined (MC3 “maintenance”) is used for the discontinuous feeding, or maintenance, of the fermentation with the carbonaceous sources of interest at a rate calibrated according to the growth of the microorganism.

The composition in grams per liter of the three media is described in Table 19

TABLE 19

| | MC1 | MC2 | MC3 |
|---|---|---|---|
| | « inoculum » | « batch » | « maintenance » |
| (NH₄)₂SO₄ | 4.7 | 4.7 | |
| Na₂HPO₄; 7H₂O | 12 | 9 | |
| KH₂PO₄ | 2.7 | 2.03 | |
| MgSO₄; 7H₂O | 0.8 | 1.03 | |
| Nutrient Broth | 3 | / | |
| Nonanoic acid | / | 0.98 | 980 |
| Undecylenic acid | | 0.02 | 20 |
| Microelement solution | / | 10 | |
| Acrylic acid | / | / | |

| | |
|---|---|
| NaOH 2N | QSP pH = 6.8 |
| MilliQ water | QSP m = 1000 g |

Table 19: Composition in grams per liter of culture media for preculture and maintenance.

The composition of Nutrient Broth in mass percentage is 37.5% beef extract and 62.5% peptone. Reference 233000 DIFCO™.

The composition of the solution of microelements in grams per liter is described in Table 20:

TABLE 20

| | | |
|---|---|---|
| FeSO₄•7H₂O | 10.0 | g |
| CaCl₂•2H₂O | 3.0 | g |
| ZnSO₄•7H₂O | 2.2 | g |
| MnSO₄•4H₂O | 0.5 | g |
| H₃BO₃ | 0.3 | g |
| CoCl₂•6H₂O | 0.2 | g |
| Na₂MoO₄•2H₂O | 0.15 | g |
| NiCl₂•6H₂O | 0.02 | g |
| CuSO₄•5H₂O | 1.00 | g |

| | |
|---|---|
| MilliQ water (or HCl 0.5N) | QSP 1000 g |

Table 20: composition in grams per liter of the solution of microelement

100 mL of preculture are prepared by suspending a cryotube containing 1 mL of the strain with 100 mL “inoculum” culture media at pH adjusted to 6.8 with 2N NaOH in a 250 mL Fernbach flask then incubating at 30° C. at 150 rpm for 24 hours. 1.9 L of MC2 “BATCH” culture medium placed in a previously sterilized 3 L chemostat are inoculated at OD=0.1 with the 100 mL of preculture. After 4 hours at 30° C. at 850 rpm, the introduction of the maintenance is carried out by applying the flow rate defined by equation 1. At the end of the introduction, the biomass is isolated by centrifugation then washed three times with some water. The biomass is dried by freeze-drying before being extracted with dichloromethane for 24 hours. The suspension is clarified by filtration on a GF/A filter (Wattman®) the filtrate, composed of PHA in solution in dichloromethane, is concentrated by evaporation then dried under high vacuum at 40° C. until constant mass. The PHA can optionally be purified by solubilization and successive precipitations such as a dichloromethane methanol system for example.

The PHA was characterized by gas chromatography equipped with an FID detector. It conforms to the expected chemical structure, with an unsaturation rate of 2%.

### Example 11′: Functionalization of mcl-PHA with Linear Side Chain R¹ Representing a n-Octylenyl Group and R² n-Hexyl Unsaturated at 2% of Example 1″ with Thiolactic Acid

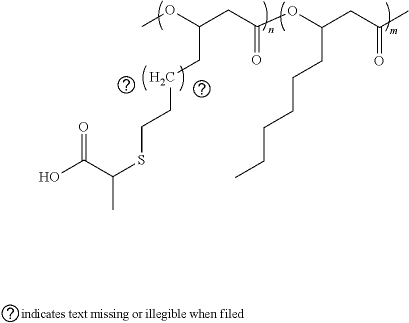

2 g of compound of example 1″ and 180 mg of thiolactic acid were dissolved in 15 mL of ethyl acetate at room temperature with stirring. 5 mg of 2-Hydroxy-2-methylpropiophenone (HMP) was added to the mixture. The medium was then irradiated under a 100 W UV lamp at 365 nm (reference) and with stirring for at least 10 minutes. The reaction medium thus obtained is poured onto a Teflon plate, then dried under dynamic vacuum at 40° C., to obtain a homogeneous film. The PHA grafted with thiolactic acid was fully characterized by proton NMR. The proton NMR spectrum shows that the characteristic signals of the unsaturations have completely disappeared.

Compositions of example 32 to 35 comprise ingredients according to table 21

TABLE 21

| | | | | Example 35 |
|---|---|---|---|---|
| | | | | (comparative |
| Ingredient | Example 32 | Example 33 | Example 34 | with I′Ex. 34) |
| PHA of Example 21 | | | 20 | |
| PHA of Example 1″″ a) | 10 | | | |
| PHA of Example 12 a) | | 10 | | |
| PHB-HV (comparative) | | | | 20 |
| Poly(3-hydroxybutyric | | | | |
| acid-co-3- | | | | |
| hydroxyvaleric acid) | | | | |
| natural origin, PHV | | | | |
| content 8 mol % | | | | |
| Sunflower wax | 1 | 2 | 5 | 5 |
| Helianthus annulus | | | | |
| (sunflower seed wax) | | | | |
| Stearyl alcool | | | 1 | 1 |
| Isododecane | | 87.85 | 71.5 | 71.5 |
| Parleam - | 0.2 | | | |
| Hydrogenated | | | | |
| isoparaffine (6-8 moles | | | | |
| og isobutylene) | | | | |
| Isononyl isononanoate | | 0.15 | | |
| Cetiol UT - Mixture of | 89.8 | | | |
| undecane and | | | | |
| tridecane | | | | |
| Ethanol | | | 2.5 | 2.5 |

### Wear Resistance

### Description of the Test:

The first step in this test consists in making a deposit. The deposits are prepared on a Byko Chart Lenata contrast card and left to dry for 24 hours at 25° C. and 45% RH. The final thickness of the deposit is 30 μm.

A wear resistance test is performed on this dry deposit. A hydrophilic steel ball is used as a friction device. The load or normal force applied is 1N, and the displacement speed is 50 mm·s⁻¹. On each film are defined tracks on which the friction device makes multiple passes. In the case of wear measurements, permanent contact is maintained during the to and fro trips of the ball on the deposit. The number of passes is increased for each track. The wear resistance is quantified as the minimum number of passes to completely wear out the deposit.

In the case of this study, the number of passes per track are, respectively, 10, 30, 50, 100, 200 and 300 passes.

Each measurement was repeated five times.

The results of the wear resistance tests are quantified as described in the table below:

TABLE 22

| Wear resistance | Evaluations |
|---|---|
| 0-10 passes of the ball | −− |
| 10-30 passes of the ball | − |
| 30-50 passes of the ball | + |
| 50-100 passes of the ball | ++ |
| 100-200 passes of the ball | +++ |
| 200-300 passes of the ball | ++++ |
| >300 passes of the ball | +++++ |

TABLE 22

| Exemples | Wear resistance |
|---|---|
| Example 32 | +++++ |
| Example 33 | +++++ |
| Example 34 | +++ |
| Example 35 | No deposit |
| (comparative with example. 34) | |

### Results

An improvement in dry wear resistance was observed when adding wax compared to an mcl-PHA formulated alone. It should also be noted that a formula made from an scl-PHA does not lead to a deposit. It should be noted that for example 35 carried out with scl-PHA, it was not possible to obtain a deposit.

It is seen that the compositions of the invention (Examples 32 to 34) remain very water-resistant. The presence of several oils has no impact on the water resistance of the films obtained.

1. A composition C1′ comprising:
a) one or more polyhydroxyalkanoate (PHA) copolymers which contain at least two different repeating polymer units chosen from the units (A) and (B) below, and also the optical or geometrical isomers thereof, the organic or mineral acid or base salts thereof, and the solvates thereof: 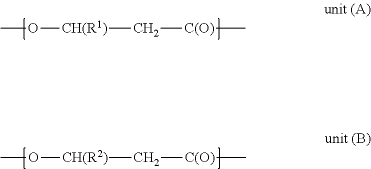
in which polymer units (A) and (B):
R¹ represents a saturated or unsaturated, linear or branched, non-cyclic hydrocarbon-based chain, or a saturated or unsaturated, aromatic or non-aromatic cyclic hydrocarbon-based chain, comprising from 5 to 28 carbon atoms;
said hydrocarbon-based chain being:
optionally substituted with one or more atoms or groups chosen from: a) halogen, b) hydroxyl, c) thiol, d) (di)(C₁-C₄)(alkyl)amino, e) (thio)carboxy, f) (thio)carboxamide —C(O)—N(Rₐ)₂ or C(S)—N(Rₐ)₂, g) cyano, h) iso(thio)cyanate, i) (hetero)aryl, and j) (hetero)cycloalkyl, k) cosmetic active agent; l) R—X with R representing a group chosen from α) cycloalkyl, β) heterocycloalkyl γ) (hetero)aryl, δ) cosmetic active agent, m) thiosulfate, and X representing a′) O, S, N(Rₐ) or Si(R_{b})(R_{c}), b′) S(O)ᵣ, or (thio)carbonyl, c′) or combinations of a′) with b′); Rₐ representing a hydrogen atom, or a (C₁-C₄)alkyl group or an aryl(C₁-C₄)alkyl group; R_{b} and R_{c}, which may be identical or different, represent a (C₁-C₄)alkyl or (C₁-C₄)alkoxy group and/or
optionally interrupted with one or more a′) heteroatoms, b′) S(O)ᵣ, (thio)carbonyl, c′) or combinations of a′) with b′) with r being equal to 1 or 2, Rₐ being as defined previously; R_{b} and R_{c} being as defined previously; and
R² represents a cyclic or non-cyclic, linear or branched, saturated or unsaturated hydrocarbon-based group comprising from 3 to 30 carbon atoms optionally substituted with one or more atoms or groups a) to m) and/or optionally interrupted with one or more heteroatoms or groups a′) to c′) as defined for R¹; and
b) one or more fatty substances which are crystallizable at 25° C. and at atmospheric pressure; and
c) optionally one or more oils, being fatty substances that are liquid at 25° C. and at atmospheric pressure; and
d) optionally one or more organic solvents;
e) optionally water;
it being understood that:
(A) is different from (B).

2. The composition C1′ according to claim 1, in which the PHA copolymer(s) a) contain the repeating unit of formula (I), and also the optical or geometrical isomers thereof, the organic or mineral acid or base salts thereof, and the solvates thereof: 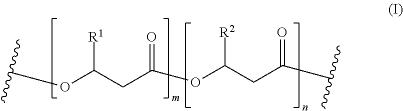
in which formula (I):
m and n are integers greater than or equal to 1.

3. The composition C1′ according to claim 1, in which the PHA copolymer(s) a) contain three different repeating polymer units (A), (B) and (C) below, and also the optical or geometrical isomers thereof and the solvates thereof: 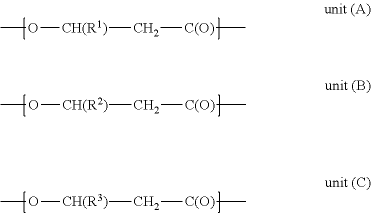
in which polymer units (A), (B) and (C):
R³ represents a saturated or unsaturated, linear or branched, cyclic or non-cyclic, hydrocarbon-based group comprising from 1 to 30 carbon atoms, optionally substituted with one or more atoms or groups a) to m) and/or optionally interrupted with one or more heteroatoms or groups a′) to c′) as defined for R¹; and
it being understood that:
(A) is different from (B) and (C), (B) is different from (A) and (C), and (C) is different from (A) and (B);

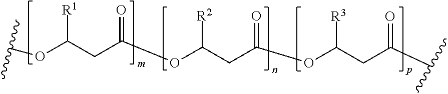

4. The composition C1′ according to claim 1, in which the PHA copolymer(s) a) contain four different repeating polymer units (A), (B), (C) and (D) below, and also the optical or geometrical isomers thereof, the organic or mineral acid or base salts thereof, and also the solvates thereof: 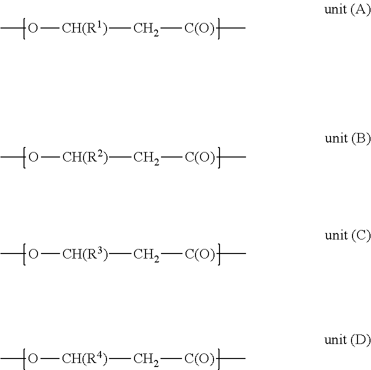
in which polymer units (A), (B), (C) and (D):
R₄ represents a cyclic or non-cyclic, linear or branched, saturated hydrocarbon-based group comprising from 3 to 30 carbon atoms optionally substituted with one or more atoms or groups a) to m) and/or optionally interrupted with one or more heteroatoms or groups a′) to c′) as defined for R¹:
(A) is different from (B), (C) and (D), (B) is different from (A), (C) and (D), (C) is different from (A), (B) and (D), and (D) is different from (A), (B) and (C

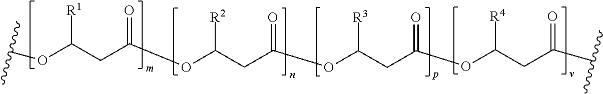

5. The composition C1′ according to claim 1, in which the PHA copolymer(s) a) contain five different repeating polymer units (A), (B), (C), (D) and (E) below, and also the optical or geometrical isomers thereof, the organic or mineral acid or base salts thereof, and also the solvates thereof: 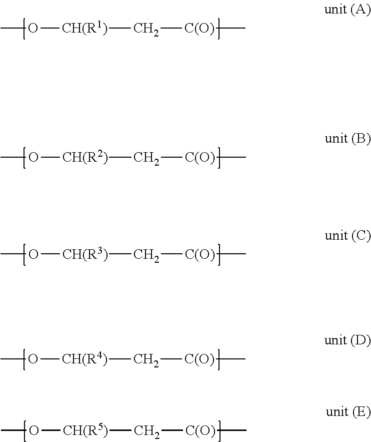
in which polymer units (A), (B), (C), (D) and (E):
R⁵ represents a cyclic or non-cyclic, linear or branched, saturated hydrocarbon-based group comprising from 3 to 30 carbon atoms optionally substituted with one or more atoms or groups a) to m) and/or optionally interrupted with one or more heteroatoms or groups a′) to c′) as defined for R¹;
it being understood that:
(A) is different from (B)(C), (D) and (E); (B) is different from (A), (C), (D) and (E); (C) is different from (A), (B), (D) and (E); (D) is different from (A) (B), (C) and (E); and (E) is different from (A), (B), (C) and (D).

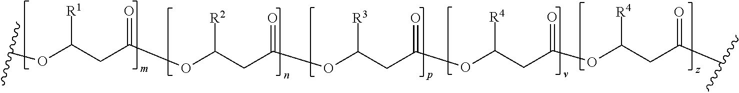

6. The composition C1′ according to claim 1, in which R¹ represents a linear or branched (C₅-C₂₈)alkyl hydrocarbon-based chain.

7. The composition C1′ according to claim 1, in which R¹ represents a hydrocarbon-based chain, being C₇-C₂₀ alkyl, which is interrupted with one or more atoms or groups chosen from O, S, N(Rₐ), carbonyl, or combinations thereof.

8. The composition C1′ according to claim 1, in which R¹ has the following formula —(CH₂)ᵣ—X-(ALK)ᵤ-G with X being as defined previously, ALK represents a linear or branched (C₁-C₁₀)alkylene chain, r represents an integer inclusively between 6 and 11; u is equal to 0 or 1; and G represents a hydrogen atom or a group chosen from hydroxyl, carboxyl, (di)(C₁-C₄)(alkyl)amino, (hetero)aryl, cycloalkyl, or a sugar.

9. The composition C1′ according to claim 1, in which the PHA copolymer(s) a) include a radical R² chosen from linear or branched (C₁-C₂₈)alkyl, and linear or branched (C₂-C₂₈)alkenyl.

10. The composition C1′ according to claim 1, in which the PHA copolymer(s) a) include a radical R² which is a linear or branched, preferably linear, (C₁-C₈)alkyl group; or R² is a branched (C₃-C₈)alkyl group.

11. The composition C1′ according to claim 1, in which the PHA copolymer(s) are such that, when R¹ represents a (C₃-C₃₀)alkyl group, the unit (A) is present in a molar percentage ranging from 0.5% to 99%; the unit (B) is present in a molar percentage ranging from 1% to 99.5%; and unit (C) is present in a molar percentage ranging from 0.5% to 20% relative to all of the units (A), (B) and (C).

12. The composition C1′ according to claim 1, in which the PHA copolymer(s) a) are such that when R¹ represents a hydrocarbon-based chain chosen from i) linear or branched (C₅-C₂₈)alkyl, ii) linear or branched (C₅-C₂₈)alkenyl, iii) linear or branched (C₅-C₂₈)alkynyl, the hydrocarbon-based group is linear, said hydrocarbon-based chain being substituted with one or more atoms or groups a) to m) and/or interrupted with one or more heteroatoms or groups a′) to c′) as defined for R¹; it in particular represents a hydrocarbon-based group chosen from linear or branched (C₄-C₂₈)alkyl, optionally substituted with one or more atoms or groups a) to m) and/or interrupted with one or more heteroatoms or groups a′) to c′) as defined previously,
the unit (A) is present in a molar percentage ranging from 0.1% to 99%; and
the unit (B) is present in a molar percentage ranging from 1% to 99.9%, preferentially a molar percentage from 2% to 99%; and
the unit (C) is present in a molar percentage ranging from 0.5 to 20%, based on the total of units (A), (B) and (C).

13. The composition C1′ according to claim 1, in which the PHA copolymer(s) comprise the following repeating units (A), and also the optical or geometrical isomers thereof, the organic or mineral acid or base salts thereof, and the solvates thereof:

| (A) | R¹ |
|---|---|
| A1 | -ALK₁-S-ALK₂-COOH |
| A2 | -ALK₁-S-ALK₂-H |
| A3 | -ALK₁-S-ALK₂-OH |
| A4 | -ALK₁-S-ALK₂-NH₂ |
| A5 | -ALK₁-S-Cycl′ |
| A6 | -ALK₁-S-CH₂-Fur |
| A7 | -ALK₁-S-Sug |
| A8 | -ALK₁-S-ALK₂-Ar |
| A9 | -ALK₁-Hal |
| A10 | -ALK₁-CN |
| A11 | -ALK₁-CH═CRᵣR_{w} |
| A12 | -ALK₂-H |

in which repeating units A1 to A12:
ALK₁ represents a divalent linear or branched C₁-C₂₀, hydrocarbon-based radical;
ALK₂ represents a divalent linear or branched C₁-C₂₀, hydrocarbon-based radical;
Rr and Rw independently denote a hydrogen atom or a C₁-C₄ alkyl radical;
Hal represents a halogen atom;
Ar: represents a (hetero)aryl group;
Cycl′: represents a cycloalkyl group;
Fur: represents a furyl group;
Sug: represents a sugar group.

14. The composition C1′ according to claim 1, in which the PHA copolymer(s) a) comprise the following repeating units, and also the optical or geometrical isomers thereof, the organic or mineral acid or base salts thereof, and the solvates thereof.

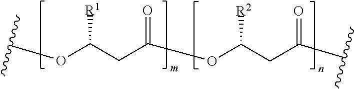

| Compounds | R¹ | R² |
|---|---|---|
| (1) | —(CH₂) [symbol] —S—CH(CH₃)—C(O)—OH | —(CH₂)₄—CH₃ |
| (2) | —(CH₂)₈—S—(CH₂)₇—CH₃ | —(CH₂)₄—CH₃ |
| (3) | —(CH₂)₈—S—(CH₂)₈—OH | —(CH₂)₄—CH₃ |
| (4) | —(CH₂)₈—S—(CH₂)₂—NH₂ | —(CH₂)₄—CH₃ |
| (5) | —(CH₂)₈—S-Cycl | —(CH₂)₄—CH₃ |
| (6) | —(CH₂)₈—S—CH₂-Fur | —(CH₂)₄—CH₃ |
| (7) | —(CH₂)₈—S-Sug | —(CH₂)₄—CH₃ |
| (8) | —(CH₂) [symbol] —S—(CH₂)₂—Ar | —(CH₂)₄—CH₃ |
| (9) | —(CH₂)₈—S—CH₂—Ar′ | —(CH₂)₄—CH₃ |
| (10) | —(CH₂)₈—S—CH(CH₃)—C(O)—OH | —(CH₂)₅—CH₃ |
| (11) | —(CH₂)₅-Hal | —(CH₂)₅—CH₃ |
| (12) | —(CH₂)₃—CN | —(CH₂) [symbol] —CH₃ |
| (13) | 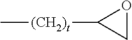 | —(CH₂)₅—CH₃ |
| (14) | —(CH₂)₂—Ar | —(CH₂)₅—CH₃ |
| (15) | —(CH₂)₄—CH₃ | —(CH₂)₂—CH₃ |
| (16) | —(CH₂)₅—CH₃ | —(CH₂)₃—CH₃ |
| (17) | —(CH₂)₆—CH₃ | —(CH₂)₄—CH₃ |
| (18) | —(CH₂)₈—CH₃ | —(CH₂)₆—CH₃ |
| (19) | —(CH₂)₃—CH(CH₃)CH₃ | —CH₂—CH(CH₃)CH₃ |
| (20) | —(CH₂)₆—CH═CH₂ | —(CH₂) [symbol] —CH₃ |
| (21) | —(CH₂)₂—CH═C(CH₃)CH₃ | —CH₂—CH(CH₃)CH₃ |
| (2a) | —(CH₂)₈—S—(CH₂)₇—CH₃ | (CH₂)₅—(CH₃) |

[symbol]

indicates data missing or illegible when filed
m and n are as defined previously, Hal represents a halogen atom and t represents an integer between 1 and 10;
Ar: represents a (hetero)aryl group such as phenyl;
Ar′: represents a (C₁-C₄)alkyl(hetero)aryl group;
Cycl: represents a cyclohexyl group;
Fur: represents a furyl group;
Sug: represents a sugar group

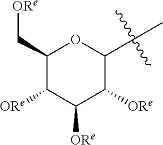

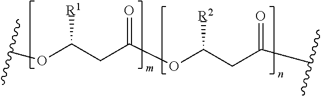

| Compounds | R¹ | R² |
|---|---|---|
| (1′) | —(CH₂) [symbol] —S—CH(CH₃)—C(O)—OH | —(CH₂)₄—CH₃ |
| (2′) | —(CH₂)₈—S—(CH₂)₇—CH₃ | —(CH₂)₄—CH₃ |
| (3′) | —(CH₂) [symbol] —S—(CH₂)₆—OH | —(CH₂)₄—CH₃ |
| (4′) | —(CH₂)₈—S—(CH₂)₂—NH₂ | —(CH₂)₄—CH₃ |
| (5′) | —(CH₂)₈—S-Cycl | —(CH₂)₄—CH₃ |
| (6′) | —(CH₂)₈—S—CH₂-Fur | —(CH₂)₄—CH₃ |
| (7′) | —(CH₂)₈—S-Sug | —(CH₂)₄—CH₃ |
| (8′) | —(CH₂)₈—S—(CH₂)₂—Ar | —(CH₂)₄—CH₃ |
| (9′) | —(CH₂) [symbol] —S—CH₂—Ar′ | —(CH₂)₄—CH₃ |
| (10′) | —(CH₂)₈—S—CH(CH₃)—C(O)—OH | —(CH₂)₃—CH₃ |
| (11′) | —(CH₂)₅-Hal | —(CH₂) [symbol] —CH₃ |
| (12′) | —(CH₂)₃—CN | —(CH₂)₃—CH₂ |
| (13′) | 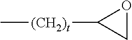 | —(CH₂) [symbol] —CH₃ |
| (14′) | —(CH₂)₂—Ar | —(CH₂)₃—CH₃ |
| (15′) | —(CH₂)₄—CH₃ | —(CH₂)₂—CH₃ |
| (16′) | —(CH₂)₃—CH₃ | —(CH₂)₃—CH₃ |
| (17′) | —(CH₂)₆—CH3 | —(CH₂)₄—CH₃ |
| (18′) | —(CH₂)₈—CH₃ | —(CH₂)₆—CH₃ |
| (19′) | —(CH₂)₃—CH(CH₃)CH₃ | —CH₂—CH(CH₃)CH₃ |
| (20′) | —(CH₂)₈—CH═CH₂ | —(CH₂)₃—CH₃ |
| (21′) | —(CH₂)₂—CH═C(CH₃)CH₃ | —CH₂—CH(CH₃)CH₃ |
| (22′) | —(CH₂)₄—CH₃ | —(CH₂)₂—CH₃ |
| (23′) | —(CH₂)₅—CH₃ | —(CH₂)₃—CH₃ |
| (24′) | —(CH₂)₆—CH₃ | —(CH₂)₄—CH₃ |

[symbol]

indicates data missing or illegible when filed

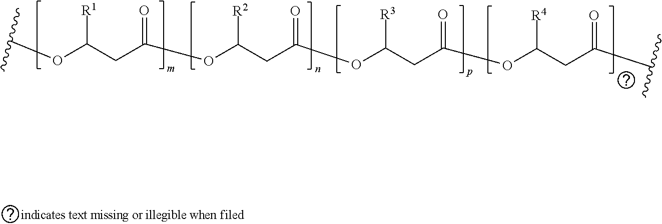

| Compounds | R¹ | R² | R³ | R⁴ |
|---|---|---|---|---|
| (25) | —(CH [symbol] ) [symbol] —CH [symbol] | —(CH [symbol] ) [symbol] —CH [symbol] | —(CH [symbol] ) [symbol] —CH [symbol] | —(CH [symbol] ) [symbol] —CH [symbol] |
| (26) | —(CH [symbol] ) [symbol] —CH [symbol] | —(CH [symbol] ) [symbol] —CH [symbol] | —(CH [symbol] ) [symbol] —CH [symbol] | —CH [symbol] |
| (27) | —(CH [symbol] ) [symbol] —CN | —(CH [symbol] ) [symbol] —CH [symbol] | —(CH [symbol] ) [symbol] —CH [symbol] | —CN |
| (28) | —(CH [symbol] ) [symbol] —Ar | —(CH [symbol] ) [symbol] —CH [symbol] | —(CH [symbol] ) [symbol] —CH [symbol] | —Ar |

[symbol]

indicates data missing or illegible when filed

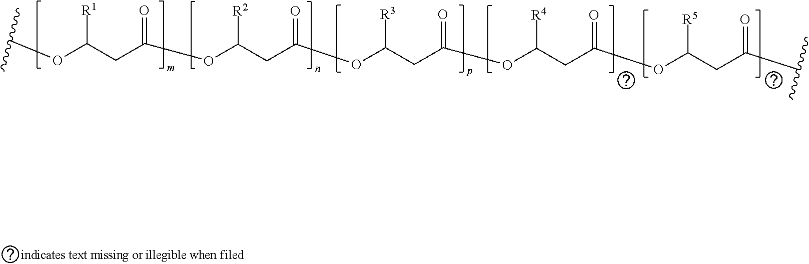

| Com- | | | | | |
|---|---|---|---|---|---|
| pounds | R¹ | R² | R³ | R⁴ | R⁵ |
| (29) | —(CH₂) [symbol] —CH═CH₂ | —(CH₂) [symbol] —CH₃ | —(CH₂) [symbol] —CH₃ | —(CH₂)₇—CH═CH₂ | —(CH₂) [symbol] —CH═CH₂ |
| (30) | —(CH₂) [symbol] —CH═CH₃ | —(CH₂)₄—CH₃ | —(CH₂)₂—CH₃ | —(CH₂)₇—CH═CH₂ | —(CH₂) [symbol] —CH═CH₂ |
| (31) | —(CH₂)₈—CH₃ | —(CH₂) [symbol] —CH₃ | —(CH₂)₄—CH₃ | —(CH₂)₂—CH₃ | —CH₃ |
| (32) | —(CH₂)₁₈—CH₃ | —(CH₂)₆—CH₃ | —(CH₂)₆—CH₃ | —(CH₂)₄—CH₃ | —(CH₂)₂—CH₃ |
| (33) | —(CH₂)₈—S—CH | —(CH₂)₄—CH₃ | —(CH₂)₂—CH₃ | —(CH₂) [symbol] —S—CH | —(CH₂)₄—S—CH |
| | (CH₃)—C(O)—OH | | | (CH₃)—C(O)—OH | (CH₃)—C(O)—OH |
| (34) | —(CH₂)₈—S— | —(CH₂)₄—CH₃ | —(CH₂)₂—CH₃ | —(CH₂)₆—S— | —(CH₂)₄—S— |
| | (CH₂)₇—CH₃ | | | (CH₂)₇—CH₃ | (CH₂)₇—CH₃ |
| (35) | —(CH₂)₈—S— | —(CH₂)₄—CH₃ | —(CH₂)₂—CH₃ | —(CH₂)₆—S— | —(CH₂)₄—S— |
| | (CH)₈—OH | | | (CH₂)₆—OH | (CH₂)₈—OH |
| (36) | —(CH₂)₈—S— | —(CH₂) [symbol] —CH₃ | —(CH₂)₂—CH₃ | —(CH2)₆—S— | —(CH₂)₄—S— |
| | (CH₂)₂—NH₂ | | | (CH₂)₂—NH₂ | (CH₂)₂—NH₂ |
| (37) | —(CH₂)₈—S-Cycl | —(CH₂)₄—CH₃ | —(CH₂) [symbol] —CH₃ | —(CH₂)₈—S-Cycl | —(CH₂)₄—S-Cycl |
| (38) | —(CH₂)₈—S—CH₂- | —(CH₂)₄—CH₃ | —(CH₂)₂—CH₃ | —(CH₂) [symbol] S—CH₂-Fur | —(CH₂)₄—S—CH₂-Fur |
| | Fur | | | | |
| (39) | —(CH₂)₈—S-Sug | —(CH₂)₄—CH₃ | —(CH₂)₂—CH₃ | —(CH₂)₈—S-Sug | —(CH₂)₄—S-Sug |
| (40) | —CH₂)₈—S—(CH₂)₂—Ar | —(CH₂)₄—CH₃ | —(CH₂)₂—CH₃ | —(CH₂)₆—S—(CH₂)₂—Ar | —(CH₂)₄—S—(CH₂)₂—Ar |
| (41) | —(CH₂)₈—S—CH₂—Ar′ | —(CH₂)₄—CH₃ | —(CH₂)₂—CH₃ | —(CH₂)₈—S—CH₂—Ar′ | —(CH₂)₄—S—CH₂—Ar′ |
| (42) | —(CH₂)₈—S—CH | —(CH₂)₈—CH₃ | —(CH₂)₃—CH₃ | —(CH₂)₈—S—CH | —(CH₂)4—S—CH |
| | (CH₃)—C(O)—OH | | | (CH₃)—C(O)—OH\| | (CH₃)—C(O)—OH— |
| (43) | —(CH₂)₈-Hal | —(CH₂)₅—CH₃ | —(CH₂)₃—CH₃ | —(CH₂)₃-Hal | (CH₂)-Hal |
| (44) | 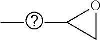 | —(CH₂)₅—CH₃ | —(CH₂)₃—CH₃ | 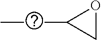 | 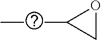 |

[symbol]

indicates data missing or illegible when filed

15. The composition C1′ according to claim 1 ranging from 0.1% to 60% by weight relative to the total weight of the composition.

16. The composition C1′ according to claim 1, in which b) the crystallizable fatty substance(s) are chosen from:
i) crystallizable fatty substances of fossil origin;
ii) crystallizable fatty substances of plant origin,
iii) fatty substances of animal origin.

17. The composition C1′ according to claim 1, in which b) the crystallizable fatty substance(s) are chosen from crystallizable mono/di/triesters derived from C₁₀-C₃₆ fatty alcohols and/or C₁₂-C₃₈ fatty acids, and mixtures thereof.

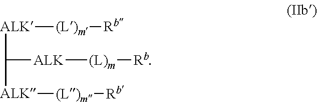

18. The composition C1′ according to claim 1, in which b) the crystallizable fatty substance(s) are present in composition C1′ in an amount of between 0.01% and 30% by weight relative to total weight of the composition.

19. The composition C1′ according to claim 1, which comprises c) one or more fatty substances.

20. The composition C1′ according to claim 1, which comprises c) on e or more fatty substances chosen from:
plant oils formed by fatty acid esters of polyols;
linear, branched or cyclic esters containing more than 6 carbon atoms;
hydrocarbons;
ethers containing 6 to 30 carbon atoms;
ketones containing 6 to 30 carbon atoms
aliphatic fatty monoalcohols containing 6 to 30 carbon atoms, the hydrocarbon-based chain not including any substitution groups;
polyols containing 6 to 30 carbon atoms, such as hexylene glycol; and
mixtures thereof.

21. The composition C1′ according to claim 1, comprising d) one or more organic solvents other than c) polar protic or polar aprotic organic solvents; preferably, the amount of organic solvent(s) is less than 70% by weight relative to the total weight of composition C1′.

22. The composition C1′ according to claim 1, which also comprises one or more colouring agents chosen from pigments, direct dyes and mixtures thereof.

23. A process for treating keratin materials by applying the composition as defined in claim 1.

24. A process for treating keratin materials selected from the group α) keratin fibres and β) human skin by applying to the keratin materials the composition according to claim 1.
